(19) United States
(12) Patent Application Publication
(10) Pub. No.: US 2012/0108492 A1
(43) Pub. Date: May 3, 2012

(54) LIGAND-SPECIFIC NON-ANTIBODY COMPOUNDS THAT INHIBIT CR2 ACTIVATION AND METHODS OF USE THEREOF

(75) Inventors: V. Michael Holers, Denver, CO (US); Jonathan P. Hannan, Northern Ireland (GB); James Kovacs, Monument, CO (US)

(73) Assignee: The Regents of the University of Colorado, Denver, CO (US)

(21) Appl. No.: 13/119,706

(22) PCT Filed: Sep. 18, 2009
(86) PCT No.: PCT/US09/57580

§ 371 Date: Sep. 6, 2011

Related U.S. Application Data
(60) Provisional application No. 61/098,708, filed on Sep. 19, 2008.

(51) Int. Cl. A61K 38/02; C07K 7/06; A61P 31/22; C07K 2/00

(52) U.S. Cl. 514/1.1; 530/300; 530/328

(57) ABSTRACT

The present invention describes a novel non-antibody ligand-specific compound that selectively binds to a complement receptor type 2 (CR2) protein, a ligand thereof, or both, wherein the compound competitively inhibits CR2 ligand's binding to a CR2 protein in a standard assay. The present invention also describes compositions and methods of use thereof.

### CROSS-REFERENCE TO RELATED APPLICATIONS

This application claims priority to U.S. Provisional Patent Application No. 61/098,708, filed Sep. 19, 2008, the disclosure of which is incorporated herein by reference in its entirety.

### STATEMENT OF RIGHTS TO INVENTIONS MADE UNDER FEDERALLY SPONSORED RESEARCH

This invention was made in part with government support under Grant No. NIH R01-CA053615 awarded by the National Institutes of Health. The government has certain rights to this invention.

### TECHNICAL FIELD

The present invention relates generally to novel ligand-specific non-antibody compounds that selectively bind to a complement receptor type 2 (CR2) protein, a ligand of CR2 protein, or both, and compositions and methods of use thereof.

### BACKGROUND

Complement receptor type 2 (CR2/CD21), a member of the regulators of complement activation (RCA) family of proteins, is a 145 kDa Type I transmembrane protein composed of 1) 15 or 16 short consensus repeat (SCR) extracellular domains, also known as complement control protein (CCP) modules, 2) a 28 amino acid transmembrane domain, and 3) a short 34 amino acid intra-cellular cytoplasmic tail. See, e.g., Fujisaku et al. (1989) J. Biol. Chem. 264:2118-25. CR2 binds four classes of ligands: (1) cell-bound proteolytic fragments of complement protein C3 (e.g., iC3b, C3dg, and C3d); (2) the 350 kilodalton (kD) Epstein Barr virus surface glycoprotein EBV gp350 and/or its truncated 220 kD isoform (EBV gp350/220); (3) the low affinity IgE receptor CD23; and (4) the cytokine interferon alpha (IFN-a). See e.g., Iida et al. (1983) J. Exp. Med. 158:1021-33; Weis et al. (1984) Proc. Nat'l Acad. Sci. USA 81:881-5; Fingeroth et al. (1984) Proc. Nat'l Acad. Sci. USA, 81:4510-4; Nemerow et al. (1989) Cell 56: 369-77; Nemerow et al. (1985) J. Virol. 55:347-51; Aubry et al. (1994) J. Immunol. 152:5806-13; Aubry et al. (1992) Nature 358:505-7; Asokan et al. (2006) J. Immunol. 177:383-94; and Delcayre et al. (1991) EMBO J. 10:919-26. CR2 is found in complex with other membrane proteins, such as CD19, and plays a crucial role in establishing normal immune responses. See Martin et al. (1991) J Exp Med 174:1299-311; and Tuveson et al. (1991) J Exp Med 173: 1083-9.

The primary function of CR2 is as a B-cell co-receptor for antigen-mediated B-cell activation through signal transduction. CR2 co-ligation with surface IgM by mAbs (Luxembourg and Cooper (1994) J. Immunol. 153:4448-57), C3d covalently linked to antigen, or biotin-conjugated C3dg complexed with biotinylated anti-IgM result in increased intracellular calcium release and activation of tyrosine and MAP kinases. Co-activation of the B-cell receptor via CR2/CD19 greatly amplifies an immune response, even without adjuvant.

Structural studies of CR2 and its ligands have presented conflicting results. For example, the X-ray crystal structures of CR2 SCR1-2 and CR2 SCR1-2 in complex with C3d both show a tightly compacted V shape where SCR1 contacts SCR2, with one SCR domain forming each arm of the V shape. Prota et al. (2002) Proc. Nat'l Acad. Sci. USA 99:1064-6; Szakonyi et al. (2001) Science 292:1725-8. Further, the CR2-C3d co-crystal structure suggests that only SCR2 contacts C3d and that SCR1 and SCR2 dimerize when bound to C3d. However, the dimerization has not been shown to be physiologically relevant nor present in the solution phase. Nevertheless, solution scattering, mAb mapping, and mutagenesis studies indicate that both SCR1 and SCR2 are needed for the interaction with C3d to take place. For example, solution scattering experiments indicate that SCR1 and SCR2 are in a more extended conformation in complex with C3d, and that CR2 maintains a more extended conformation with both SCR domains physically contacting C3d (Gilbert et al. (2006) J. Mol. Biol. 362:1132-47; and Gilbert et al. (2005) J. Mol. Biol. 346:859-73). Monoclonal antibody epitope mapping studies on the interaction between CR2 and C3d in solution phase suggest that both SCR1 and SCR2 are required for interaction with C3d (Guthridge et al. (2001) J. Immunol. 167:5758-66). Computational modeling analysis, molecular dynamics (MD) and site-directed mutagenesis studies all indicate that both SCR1 and SCR2 appear to be required for CR2-C3d interactions and that the interaction is mostly charge-mediated (Morikis and Lambris (2004) J. Mol. Biol. 369:567-83; Zhang et al. (2007) J. Mol. Biol. 369:567-83; Hannan et al. (2005) J. Mol. Biol. 346:845-58; and Young et al. (2007) J. Biol. Chem. 282:36614-25). Similarly, site-directed mutagenesis studies also suggest that both SCR1 and SCR2 appear to be required for the CR2-EBV gp350/220 interaction and that both CR2-C3d and CR2-EBV gp350/220 interactions are in part charge-mediated (Young et al. (2007) J. Biol. Chem. 282:36614-25).

In view of the inconsistent structural data describing the CR2-C3d complex and the lack of information regarding the nature of the interaction between CR2 and its remaining ligands (e.g., EBV gp350/220, CD23, and IFN-α), ligand-specific compounds that could be used to study the interaction between CR2 and its ligands in vitro and in vivo would facilitate more detailed analysis of these CR2-ligand interactions. To date, there is no consensus about which residues are required for each CR2-ligand interaction and there is no method available for selectively inhibiting each CR2 ligand or CR2 itself. Anti-CR2/CD21 monoclonal antibodies that block all four ligands from binding to CR2/CD21 have been isolated, but they do not selectively block individual CR2/CD21 ligands. See e.g., Guthridge et al. (2001) J. Immunol. 167:5758-66; Asokan et al. (2006) J. Immunol. 177:383-394; and Young et al. (2007) J. Biol. Chem. 282:36614-36625. Similarly, use of available structural information derived from the CR2 crystal structure and the CR2-C3d co-crystal structure may be used to identify agonists or antagonists of the CR2 receptor (see, e.g., U.S. Pat. No. 6,820,011), but the exact location of the CR2/CD21 binding site on its corresponding ligands and the relationship between each of the ligand binding sites on the CR2 molecule itself have not been determined. Ligand-specific non-antibody compounds would therefore be useful for the study of specific CR2-ligand interactions, and would have therapeutic potential for modulation of CR2 activation.

All references, publications, and patent applications disclosed herein are hereby incorporated by reference in their entirety.

### SUMMARY OF THE INVENTION

The present invention provides non-antibody compounds (e.g., non-antibody polypeptides) that selectively bind to a complement receptor type 2 (CR2) protein, a ligand thereof, or both, wherein the non-antibody compounds (e.g., non-antibody polypeptides) competitively inhibit binding of the CR2 ligand to a CR2 short consensus repeat (SCR) domain in a standard assay. In some embodiments, the CR2 ligand is selected from a group consisting of Epstein-Barr Virus (EBV), a proteolytic fragment of complement protein C3, IFN-a, and FcεRII/CD23.

The invention also provides non-antibody polypeptides that selectively bind to a complement receptor type 2 (CR2) protein, a ligand thereof, or both, wherein the ligand is EBV, and wherein the non-antibody polypeptides competitively inhibit binding of EBV to a CR2 short consensus repeat (SCR) domain in a standard assay. In some embodiments, the ligand is the 350 kilodalton (“kD”) surface glycoprotein of Epstein Barr Virus (EBV gp350), and the non-antibody polypeptide competitively inhibits binding of EBV to a CR2 short consensus repeat (SCR) domain in a standard assay.

In some embodiments, the standard assay is selected from the group consisting of competitive enzyme-linked immunosorbent assay (ELISA), radioimmunoassay (RIA), surface plasmon resonance, fluorescence activated cell sorting (FACS), flow cytometry, nuclear magnetic resonance (NMR) spectroscopy, and analytical ultracentrifugation.

In some embodiments, the non-antibody polypeptides selectively bind to a CR2 protein, a ligand thereof, or both, the CR2 ligand is EBV gp350, and the non-antibody polypeptides competitively inhibit binding of EBV to a CR2 short consensus repeat (SCR) domain in a competitive enzyme-linked immunosorbent assay (ELISA). In some embodiments, the CR2 SCR domain comprises an SCR1 domain of CR2, an SCR2 domain of CR2, or both, and optionally a linker between the SCR1 domain and the SCR2 domain.

In some embodiments, the non-antibody polypeptides selectively bind to a CR2 protein, a ligand thereof, or both, the ligand is EBV gp350, the non-antibody polypeptides competitively inhibit binding of EBV gp350 to a CR2 SCR domain in a standard assay, and the non-antibody polypeptides selectively bind to a binding site on EBV gp350 comprising two N-terminal β-barrel domains (D1 and D2) and a linker region between D1-D2 (linker-1). In some embodiments, the non-antibody polypeptides selectively bind to a binding site on EBV gp350 comprising the following amino acid positions in a wildtype EBV gp350 sequence or their equivalent positions in a variant EBV gp350 sequence: E21, D22, E155, E201, D208, E210, and D296. In some embodiments, the binding site on EBV gp350 comprises the following amino acid positions in a wildtype EBV gp350 sequence or their equivalent positions in a variant EBV gp350 sequence: Y151, I160, and W162. In some embodiments, the binding site on EBV gp350 comprises the following amino acid positions in a wildtype EBV gp350 sequence or their equivalent positions in a variant gp350 sequence: E21, D22, E155, E201, D208, E210, D296, Y151, I160, and W162

In some embodiments, the non-antibody polypeptides selectively bind to a CR2 protein, a ligand thereof, or both, the ligand is EBV, the non-antibody polypeptides competitively inhibit binding of EBV to a CR2 SCR domain in a standard assay, and the non-antibody polypeptides selectively bind to a binding site on SCR1 of CR2 protein. In some embodiments, the binding site on SCR1 of the CR2 protein comprises the following amino acid positions in a human CR2 sequence or their equivalent positions in a non-human CR2 sequence: R13, S15, A22, R28, F35, R36, K41, D49, and D52.

In some embodiments, the non-antibody polypeptides selectively bind to a CR2 protein, a ligand thereof, or both, the ligand is EBV, the non-antibody polypeptides competitively inhibit binding of EBV to a CR2 SCR domain in a standard assay, and the non-antibody polypeptides selectively bind to both an SCR1 domain and an SCR2 domain of the CR2 protein. In some embodiments, the binding site on the SCR1 domain and the SCR2 domain of the CR2 protein comprises the following amino acid positions in a human CR2 sequence or their equivalent positions in a non-human CR2 sequence: R13, A22, R28, F35, R36, K41, D49, D52, K57, E63, K67, Y68, R83, and R89. In some embodiments, the binding site on the SCR1 domain and the SCR2 domain of the CR2 protein comprises the following amino acid positions in a human CR2 SCR sequence or their equivalent positions in a non-human CR2 SCR sequence: R13, A22, R28, K67, Y68, R83, G84, R89, and S103. In some embodiments, the non-antibody polypeptide comprises the amino acid sequence CSEGSLKGC (SEQ ID NO: 20).

The present invention also provides non-antibody polypeptides that selectively bind to a complement receptor type 2 (CR2) protein, a ligand thereof, or both, wherein the ligand is a proteolytic fragment of complement component C3 (C3 fragment), and wherein the non-antibody polypeptides competitively inhibit binding of one or more C3 fragments to a CR2 short consensus repeat (SCR) domain in a standard assay. In some embodiments, the C3 fragment comprises C3d, C3dg, or iC3b. In some embodiments, the non-antibody polypeptides competitively inhibit binding of one or more C3 fragments to a CR2 SCR domain in a competitive enzyme-linked immunosorbant assay (ELISA) assay. In some embodiments, the non-antibody polypeptides selectively bind to both SCR1 and SCR2 of the CR2 protein and optionally an inter-SCR linker. In some embodiments, the non-antibody polypeptides selectively bind to a binding site on SCR1 and SCR2 of the CR2 protein comprising the following amino acid positions in a human CR2 sequence or their equivalent positions in a non-human CR2 sequence: R13, Y16, R28, Y29, S32, T34, K48, D56, K57, Y68, R83, G84, N101, N105, S109, and R122. In some embodiments, the non-antibody polypeptides selectively bind to a binding site on SCR1 and SCR2 of the CR2 protein comprising the following amino acid positions in a human CR2 sequence or their equivalent positions in a non-human CR2 sequence: R13, Y16, T34, K48, D56, K57, Y68, R83, G84, N105, and S109.

In some embodiments, the non-antibody polypeptides selectively bind to a CR2 protein, a ligand thereof, or both, the ligand is a C3 fragment, the non-antibody polypeptides competitively inhibit binding of one or more C3 fragments to a CR2 SCR domain in a standard assay, and the non-antibody polypeptides comprise a sequence selected from the group consisting of:

a) amino acid sequence APQXLSSQYSRT (SEQ ID NO: 7), wherein X is H or A;
b) amino acid sequence APXHLSSQ (SEQ ID NO: 8), wherein X is Q or A;
c) amino acid sequence ISTSNPRX₁X₂STA (SEQ ID NO: 9), wherein X₁ is H or A, and X₂ is H or A;
d) amino acid sequence ISTSNPRH (SEQ ID NO: 10);
e) amino acid sequence IATANPRHHSTA (SEQ ID NO: 11);
f) amino acid sequence CDPKNHVHC (SEQ ID NO: 12); and
g) amino acid sequence DPKNHVH (SEQ ID NO: 13).

In some embodiments, the non-antibody polypeptides selectively bind to a CR2 protein, a ligand thereof, or both, the ligand is a C3 fragment, the non-antibody polypeptides competitively inhibit binding of one or more C3 fragments to a CR2 SCR domain in a standard assay, and the non-antibody polypeptides comprise the amino acid sequence

| | |
|---|---|
| APQHLSSQYSRT, | (SEQ ID NO: 14) |
| APQALSSQYSRT, | (SEQ ID NO: 15) |
| APQHLSSQ, | (SEQ ID NO: 16) |
| APAHLSSQ, | ((SEQ ID NO: 17) |
| ISTSNPRHHSTA, | (SEQ ID NO: 18) |
| ISTSNPRAASTA, | (SEQ ID NO: 19) |
| ISTSNPRH, | (SEQ ID NO: 10) |
| IATANPRHHSTA, | (SEQ ID NO: 11) |
| CDPKNHVHC, | (SEQ ID NO: 12) |
| or | |
| DPKNHVH. | (SEQ ID NO: 13) |

The invention further provides pharmaceutical compositions comprising one or more non-antibody compounds (e.g., non-antibody polypeptides) that selectively bind to a complement receptor type 2 (CR2) protein, a ligand thereof, or both, wherein the non-antibody polypeptides competitively inhibit binding of the CR2 ligand to a CR2 short consensus repeat (SCR) domain in a standard assay, and a pharmaceutically acceptable carrier. In some embodiments, the CR2 ligand is selected from the group consisting of EBV gp350, a proteolytic fragment of complement protein C3 (“C3 fragments”) (e.g., C3d, C3dg, or iC3b), IFN-a, and FcεRII/CD23.

The invention also provides pharmaceutical compositions comprising non-antibody polypeptides that selectively bind to a complement receptor type 2 (CR2) protein, a ligand thereof, or both, wherein the ligand is the 350 kD surface glycoprotein of Epstein-Barr Virus (EBV gp350), and wherein the non-antibody polypeptides competitively inhibit binding of EBV to a CR2 short consensus repeat (SCR) domain in a standard assay, and a pharmaceutically acceptable carrier.

The invention also provides pharmaceutical compositions comprising non-antibody polypeptides that selectively bind to a complement receptor type 2 (CR2) protein, a ligand thereof, or both, wherein the ligand is a proteolytic fragment of complement component C3 (C3 fragment), and wherein the non-antibody polypeptide competitively inhibits binding of the C3 fragments to a CR2 short consensus repeat (SCR) domain in a standard assay, and a pharmaceutically acceptable carrier.

The invention also provides methods of inhibiting or reducing a biological response mediated by a complement receptor type 2 (CR2) ligand in an individual comprising administering to the individual an effective amount of a non-antibody compound (e.g., a non-antibody polypeptide), wherein the non-antibody compound (e.g., a non-antibody polypeptide) selectively binds to a CR2 protein, a ligand thereof, or both and wherein the non-antibody compound (e.g., a non-antibody polypeptide) competitively inhibits binding of the CR2 ligand to a CR2 short consensus repeat (SCR) domain in a standard assay.

The invention further provides methods of inhibiting or reducing a biological response mediated by a complement receptor type 2 (CR2) ligand in an individual comprising administering to the individual an effective amount of a non-antibody compound (e.g., a non-antibody polypeptide), wherein the non-antibody compound (e.g., a non-antibody polypeptide) selectively binds to a CR2 protein, a ligand thereof, or both and wherein the non-antibody compound (e.g., a non-antibody polypeptide) competitively inhibits binding of the CR2 ligand to a CR2 short consensus repeat (SCR) domain in a standard assay.

In some embodiments, the biological response comprises an autoimmune response, a humoral immune response, a hypersensitivity response, an EBV infection, immunoglobulin class switching, B cell activation, T cell activation, binding of antigens to follicular dendritic cells, binding of antigens or EBV to epithelial cells, IFN-a induced mRNA changes, B cell adherence to sites of inflammation, or a CR2-dependent human immunodeficiency virus-1 (HIV-1) infection.

In some embodiments, the autoimmune response is associated with a disease selected from the group consisting of coankylosing spondylitis, autoimmune hemolytic anemia, autoimmune inner ear disease, bullous pemphigoid, celiac disease, chagas disease, chronic obstructive pulmonary disease (COPD), dermatomylositis, endometriosis, hidradenitis suppurativa, interstitial cystitis, morphea, narcolepsy, neuromyotonia, pemphigus vulgaris, pernicious anaemia, polymyositis, primary biliary cirrhosis, schizophrenia, scleroderma, vasculitis, temporal arterities, vitiligo, Wegener's granulomatosis, myasthenia gravis, Alzheimer's disease, multiple sclerosis, rheumatoid arthritis, systemic lupus erythematosus, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barré syndrome, Hashimoto's disease, idiopathic thrombocytopenic purpura, thrombotic thrombocytopenic purpura, atypical hemolytic uremic syndrome, Sjögren's syndrome, Takayasu's arteritis, and chronic fatigue syndrome.

In some embodiments, the EBV infection is associated with a disease selected from the group consisting of B-cell lymphoma, African Burkitt's lymphoma, nasopharyngeal carcinoma, EBV-associated lymphoproliferative disease, non-Hodgkin's lymphoma, oral hairy leukoplakia, a human immunodeficiency virus-1 (HIV-1) related lymphoproliferative disease, and an autoimmune disease. In some embodiments, the autoimmune disease is selected from the group consisting of systemic lupus erythematosus, rheumatoid arthritis, multiple sclerosis, and chronic fatigue syndrome.

The invention further provides for use of the compositions described herein in connection with any of the methods described herein, unless otherwise noted or as is clear from the specific context. The compositions described herein may also be used in the preparation of a medicament for use in any of the methods described herein.

In some embodiments, it is to be understood that one, some, or all of the properties of the various embodiments described herein may be combined to form other embodiments of the present invention.

### BRIEF DESCRIPTION OF THE DRAWINGS

FIGS. 1A-B. Schematic ribbon representations of human Epstein-Barr virus EBV gp350 and (B) human CR2 SCR1-2, as determined by X-ray crystallography. (A) The secondary structure of the EBV gp350 fragment corresponding to residues 4-443 of the native sequence of EBV gp350 (B95-8 strain) is shown. Indicated are the three independent β-barrel domains comprising the major structural components: Domain 1 (D1) corresponding to residues 4-153; domain 2 (D2) (residues 165-305); and domain 3 (D3) (residues 317-426). Also indicated are two structured linker regions, designated linker-1 (residues 154-164) and linker-2 (residues 306-16), connecting domains 1 and 2, and domains 2 and 3, respectively. A total of fourteen glycan moieties identified in the crystal structure are also indicated in blue. (PDB Accession Code: 2H6O). (B) The secondary structure of the two amino-terminal domains (SCR1-2) of CR2 as identified in the co-crystal structure of the CR2-C3d complex are shown (in cyan). Each SCR domain comprises five short β-strands, and four cysteine residues forming disulfide bonds at either end of the domain. Indicated are SCR1 (residues 1-62), SCR2 (residues 71-129), and the linker region connecting both domains (residues 63-70) (PDB Accession Code: 1 GHQ).

FIGS. 1C-E. (C) MBP-CR2 SCR1-2-171 mAb ELISA data. The ability of mutant forms of MBP-CR2 SCR1-2 to bind the inhibitory anti-CR2 SCR1-2 monoclonal antibody, 171. N11A, R13A, S32A, T34A, R36A, and Y64A mutants all show a greater than 20% decreased binding capacity. Data are not shown for a number of other MBP-CR2 SCR1-2 mutants which exhibited wild-type-like levels of binding (<20% reduction) binding: L10A, S15P, Y16A, R28A, K41A, K50A, K57A, K67A, Y68A, R83A, T86A, R89A and M117A (see Table 3). (D) Mapping the 171 mAb epitope onto a ribbon representation of CR2 SCR1-2. The scheme used various colors of mutants to represent the percentage of 171 mAb binding of mutants relative to wild-type CR2. (E) is the same as D, except the molecule has been rotated about the y axis by 90°. The epitope identified for mAb 171 is similar to those previously described for other inhibitory anti-CR2 monoclonal antibodies, OKB7 and FE8 (Martin et al. (1991) J. Exp. Med. 174:1299-311; Prodinger et al. (1998) J. Exp. Med. 174:1299-311). The OKB7 mAb (no longer available) epitope was previously isolated to residues 8-PILNGRIS-15 of SCR1 and the eight-residue SCR1-2 linker region 63-EYFNYKYSS-70 (Id., Martin et al., 1991). With regards to the FE8 mAb, up to five discontinuous regions of CR2 have been suggested by Western blot analysis of linear peptides to contribute to the epitope, including 63-EYFNKYS-69, corresponding to the linker region, and 16-YYSTPI-21. Martin et al. (1991) J. Exp Med. 174:1299-311. However, no additional experiments are reported to determine which of the physically discrete sites, or subset of sites, are recognized by FE8 in the three-dimensional structure of CR2 SCR1-2.

FIG. 2. MBP-CR2 SCR1-2-EBV gp350 ELISA data targeting residues within EBV gp350. The ability of MBP-CR2 SCR1-2 to bind plate-bound mutant forms of EBV gp350 (E21A, D22A, D53A, Y151A, E155A, I160A, W162A, E201A, D208A, E210A D215A, and D296A) are shown. The average and S.E.M. of the normalized values relative to wild-type EBV gp350 binding are given. Data is not shown for a number of EBV gp350 mutants which exhibited wild-type-like (<20% reduction) binding: D53A, E119A, Y159A, D163A, E214A, and E236A (see Table 1).

FIG. 3. Effect of EBV gp350 mutagenesis on wild-type CR2 binding (ELISA). (A) Alanine substitutions mapped onto a ribbon representation of the region comprising D1 and D2 of EBV gp350. The scheme used to various colors of residues shown in A and B represents the percentage binding of wild-type MBP-CR2 SCR1-2 to mutant forms of EBV gp350 (at a concentration of 5 μg/ml of mutant EBV gp350 and 2 μg/ml of wild-type MBP-CR2 SCR1-2). (B), same as for A, except the molecule has been rotated about the y axis by 60°.

FIG. 4. K562 cell-binding flow cytometry analysis of the wild-type CR2-mutant EBV gp350-biotin interaction. (A-G) Shown is the capacity of wild-type CR2 SCR1-15-expressing cell populations, which have been labeled with fluorescein isothiocyanate (FITC), to bind wild-type or mutant forms of phycoerythrin (PE)-conjugated EBV gp350-biotin. (A-F) Shown are representative whole cell populations of K562 erythroleukemia cells expressing wild-type CR2 to bind (A), wild-type (B), D22A (C), Y151A, (D), I160A (E), W162A (F), D296A forms of EBV gp350. (G) Bar chart of the normalized values of the intermediate CR2-expressing population (25%) for binding to all seventeen EBV gp350-biotin mutants generated in this study. Average and S.E. of the normalized values for the MFI (Mean Fluorescent Intensity) of the intermediate CR2-expressing population (25%) are shown. An asterisk (*) indicates that plate-bound forms of mutant EBV gp350 also exhibited decreased ability to bind MBP-CR2 SCR1-2 in ELISA study. A plus sign (+) indicates that data are inconsistent with that observed in the MBP-CR2 SCR1-2 ELISA study (see Table 1)

FIG. 5. MBP-CR2 SCR1-2-EBV gp350 ELISA data targeting residues within CR2 SCR1-2. The ability of mutant forms of MBP-CR2 SCR1-2 to bind plate-bound wild-type EBV gp350. Data are shown for R13A, S15P, R28A, R36A, K41A, K50A, K57A, K67A, R83A and R89A are shown. The average and S.E.M. of the normalized values relative to wild-type EBV gp350 binding are given. Data is not shown for a number of MBP-CR2 SCR1-2 mutants which exhibited wild-type-like levels of binding (<20% reduction) binding: L10A, N11A, Y16A, S32A, T34A, Y64A, Y68A, T86A and M117A (see Table 2)

FIG. 6. Effect of CR2 mutagenesis on wild-type EBV gp350 binding (ELISA). (A) Alanine and proline substitutions mapped onto a ribbon representation of CR2 SCR1-2. The scheme used various colors of residues shown in A and B to represent the percentage binding of wild-type EBV gp350 to mutant forms of MBP-CR2 SCR1-2 (at a concentration of 5 μg/ml EBV gp350 and 2 μg/ml of mutant MBP-CR2 SCR1-2). (B), same as for A, except the molecule has been rotated about the y axis by 90°.

FIG. 7. Ribbon representative of the HADDOCK-derived model of the CR2-EBV gp350 complex generated utilizing the mutagenesis data as described in Table 1 and Table 2, and the crystal structures of CR2 SCR1-2 and EBV gp350, respectively.

FIG. 8. Structures of CR2 and ligands of CR2. (A) X-ray crystal structure of CR2 SCR1-2. (B) X-ray co-crystal structure of CR2 SCR1-2 bound C3d. (C) X-ray and neutron scattering best-fit solution model of CR2 SCR1-2. (D) X-ray and neutron scattering best-fit co-solution model of CR2 SCR1-2 bound C3d. (E) X-ray and neutron scattering best-fit solution model of full length CR2. Green represents C3d, yellow represents CR2 SCR1, blue represents CR2 SCR2, and grey represents CR2 SCR3-15.

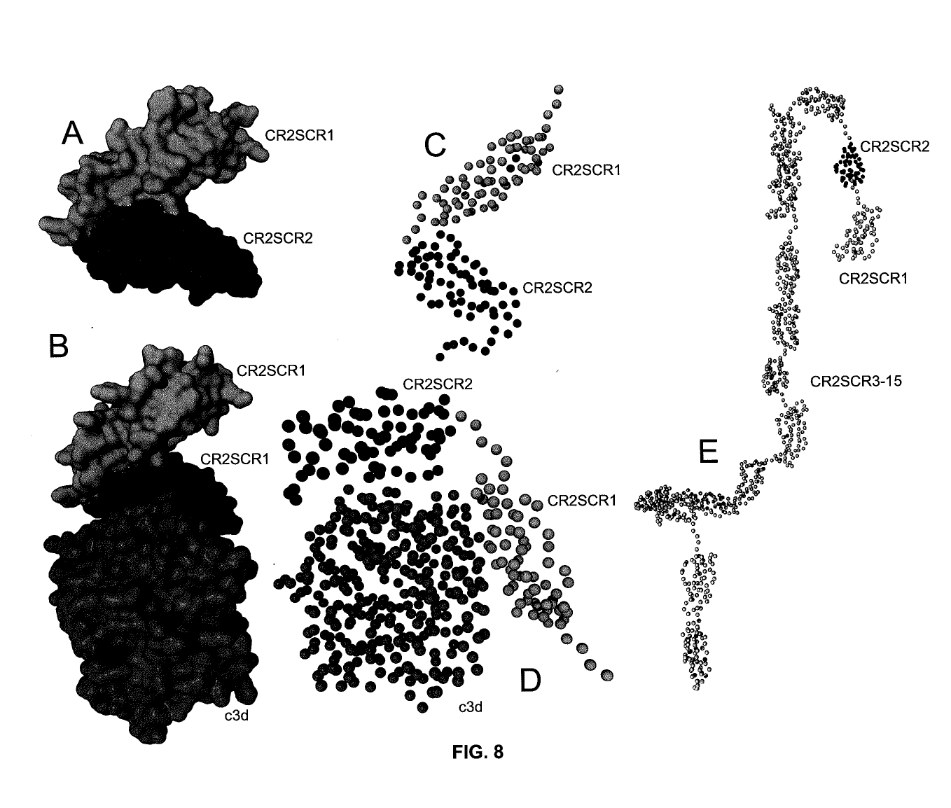

FIG. 9. CR2-C3d competition using ELISA with bound C3d and solution phase CR2 with increasing amounts of inhibitory peptides. The mean and standard deviation of at least three replicates are reported. Peptide inhibition constants were reported as IC50s in Table 4 IC50. Note that inhibition by EBV gp350 cp1 was specific to the CR2-EBV gp350 interaction (FIG. 10), and the CR2-C3d interaction was unaffected. The number system used here for CR2 is based on the reported amino acid sequence of the mature protein without the signal peptide.

FIG. 10. CR2-EBV gp350 competition using ELISA with bound EBV gp350 and solution phase CR2 with increasing amounts of inhibitory peptides. The mean and standard deviation of at least three replicates were reported. Peptide inhibition constants were reported in Table 4. Note that inhibition by C3dp1 was specific to the CR2-C3d interaction (FIG. 9), and the CR2-EBV gp350 interaction was largely unaffected.

FIG. 11. NMR titration analysis revealed that SCR1 and SCR2 of CR2 are both involved in the ligation with C3d. (A) Two super imposed ¹H-¹⁵N-TROSY-HSQC spectra of ¹⁵N-CR2 SCR1-2 (0.1 mM in ⅓×PBS) collected during titration with saturating amounts of C3d. The dark-color spectrum was CR2 SCR1-2 and the light-color spectrum was CR2 SCR1-2 with saturating amounts of C3d. (B) Surface representation of CR2 SCR1-2 X-ray crystal structure in its ligand bound state (C3d not shown) with ligand binding residues mapped out. Light-color residues represent residues unaffected by C3d titration. Dark-color residues represent residues affected by C3d titration, illustrating both SCR1 and SCR2 are required for C3d ligation.

FIG. 12. NMR titration analysis reveals that SCR1 and SCR2 of CR2 are both involved in the ligation with C3d inhibitory peptide C3dp1. A: Two super imposed ¹H-¹⁵N-TROSY-HSQC spectra of ¹⁵N-CR2 SCR1-2 (0.1 mM in ⅓×PBS) collected during titration with saturating amounts of C3dp1. The black spectrum is CR2 SCR1-2 and the red spectrum is CR2 SCR1-2 with saturating amounts of C3dp1. B: Surface representation of CR2 SCR1-2 x-ray crystal structure in its ligand bound state (C3d not shown) with ligand binding residues mapped out. Grey residues represent residues unaffected by C3dp1 titration. Yellow residues represent residues affected by C3dp1 titration, illustrating that both SCR1 and SCR2 are required for C3dp1 ligation, similar to that of C3d ligation.

FIG. 13. Surface representation of CR2 SCR1-2: Summary of binding maps. Summary of the binding maps of CR2-C3d and CR2-C3dp1. Grey residues represent residues that are not involved in either binding event. Blue residues represent residues only involved in C3d binding to CR2. Yellow residues represent residues involved in both C3d and C3dp1 binding to CR2.

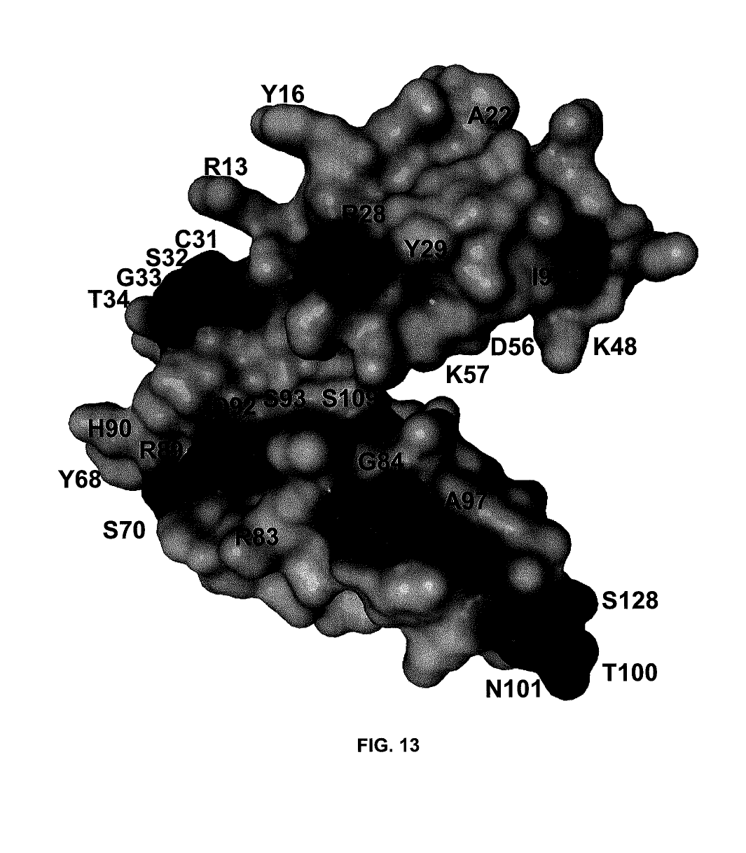

FIGS. 14A-B. (A) Titration of gp350 specific inhibitory polypeptide (gp350 cp1) with CR2 SCR1-2. (B) Surface representation of CR2 SCR1-2 X-ray crystal structure within its ligand bound state, showing the binding site of this peptide.

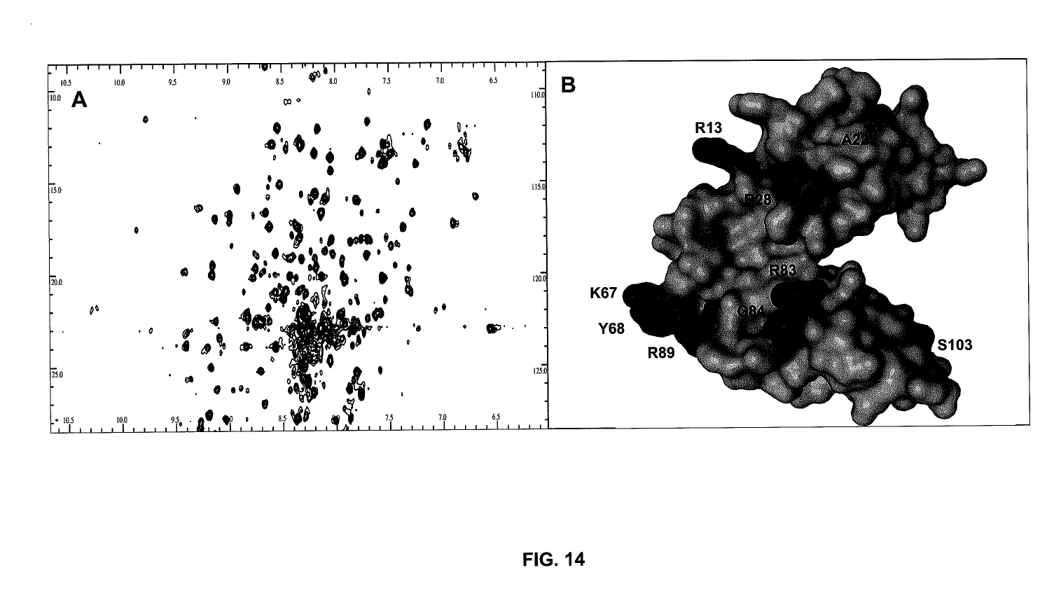

FIG. 15. Surface representation of CR2 showing sites that are shared and also unique to C3d specific inhibitory polypeptide (C3dp1) and gp350 specific inhibitory polypeptide (gp350 cp1), demonstrating ability of ligand-specific peptides to identify unique sites on CR2 for development of inhibitors. Red indicates residues affected by gp350 cp1. Yellow residues indicate residues affected by C3dp1. Blue residues indicate residues affected by both gp350 cp1 and C3dp1.

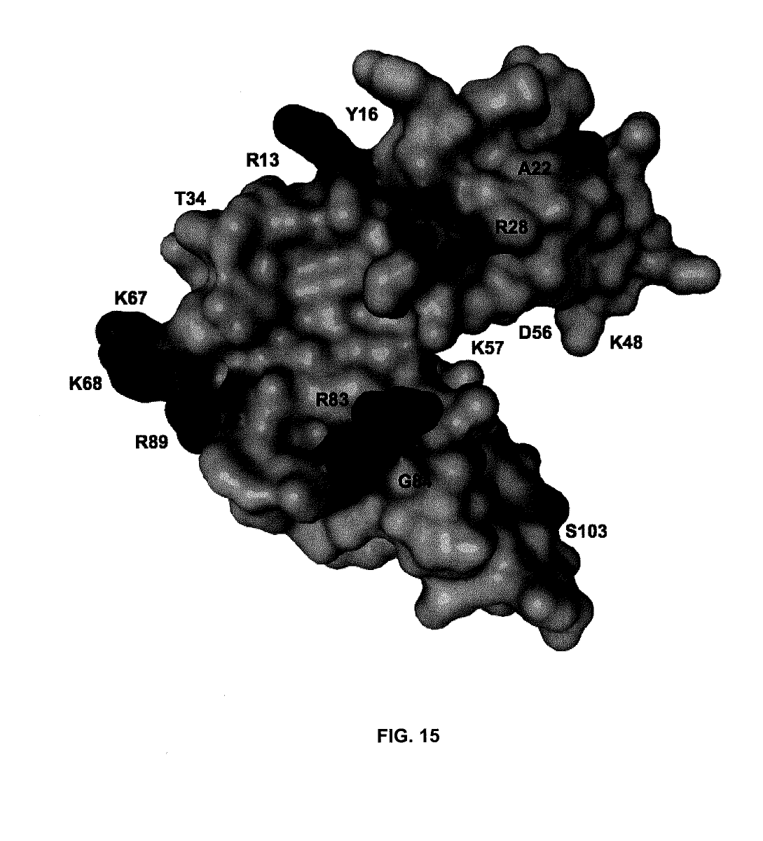

### BRIEF DESCRIPTION OF THE SEQUENCES

SEQ ID NO: 1 is the amino acid sequence of full-length human CR2 protein.

SEQ ID NO: 2 is the amino acid sequence of SCR1 of human CR2 protein.

SEQ ID NO: 3 is the amino acid sequence of SCR2 of human CR2 protein.

SEQ ID NO: 4 is the amino acid sequence of CR2 segment (SCRs1 and 2 and linker-1 human CR2 protein).

SEQ ID NO: 5 is the amino acid sequence of full-length mouse CR2 protein.

SEQ ID NO: 6 is the amino acid sequence of CR2 segment (SCRs 1 and 2 and linker-1 mouse CR2 protein).

SEQ ID NO: 7 is the amino acid sequence of APQXLSSQYSRT, wherein X is H or A.

SEQ ID NO: 8 is the amino acid sequence of APXHLSSQ, wherein X is Q or A.

SEQ ID NO: 9 is the amino acid sequence of ISTSNPRX₁X₂STA, wherein X₁ is H or A, and X₂ is H or A

SEQ ID NO: 10 is the amino acid sequence of peptide C3dp2.2.

SEQ ID NO: 11 is the amino acid sequence of peptide C3dp2.3.

SEQ ID NO: 12 is the amino acid sequence of peptide C3dcp1.

SEQ ID NO: 13 is the amino acid sequence of peptide C3dcp1.1.

SEQ ID NO: 14 is the amino acid sequence of peptide C3dp1.

SEQ ID NO: 15 is the amino acid sequence of peptide C3dp1.1.

SEQ ID NO: 16 is the amino acid sequence of peptide C3dp1.3.

SEQ ID NO: 17 is the amino acid sequence of peptide control.

SEQ ID NO: 18 is the amino acid sequence of peptide C3dp2

SEQ ID NO: 19 is the amino acid sequence of peptide C3dp2.1.

SEQ ID NO: 20 is the amino acid sequence of peptide EBV gp350 cp1.

### DETAILED DESCRIPTION

Provided herein are kits and compositions, including pharmaceutical compositions, comprising novel non-antibody polypeptides that selectively bind to a complement receptor type 2 (CR2) protein, a ligand thereof, or both. Such non-antibody polypeptides selectively bind to a CR2 protein, a ligand thereof, or both, and competitively inhibit the binding of one or more ligands to a CR2 protein. Also provided herein are methods of inhibiting or reducing a CR2 ligand-mediated biological response in an individual comprising administering to the individual an effective amount of a non-antibody polypeptide that selectively binds to a complement receptor type 2 (CR2) protein, a ligand thereof, or both, and methods of inhibiting or preventing the binding of a CR2 protein to any one or more of its cognate ligands in an individual, comprising administering to the individual an effective amount of a non-antibody polypeptide that selectively binds to a complement receptor type 2 (CR2) protein, a ligand thereof, or both. Also provided herein are methods of screening for ligand-specific non-antibody compounds (e.g., non-antibody polypeptides and other compounds) that inhibit CR2 activation, as well as compositions (e.g., pharmaceutical formulations) and articles of manufacture (e.g., kits) comprising non-antibody compounds identified by those methods of screening.

### DEFINITIONS

As used herein, the term, “non-antibody compounds” that inhibit CR2 activation may include proteins, polypeptides, oligopeptides, peptides, nucleic acids, carbohydrates, or any other molecules (e.g., structural analogs, peptidomimetics, and the like) capable of binding CR2 protein or a ligand thereof and inhibiting CR2 protein activation. In some embodiments, a non-antibody compound may bind CR2 protein or a ligand thereof and inhibit (e.g., decrease) one or more activities or functions of CR2 protein and/or a ligand thereof. For example, compounds of the present invention inhibit the binding of CR2 to its natural ligands (e.g., proteolytic fragments of complement protein C3, EBV gp350, FcεRII/CD23, and IFN-a). The term “structural analog,” as used herein, refers to a molecule that structurally or functionally resembles a molecule of interest but which has been modified in a targeted and/or controlled manner, for example, by replacing a specific substituent of the reference molecule with an alternate substituent.

As used herein, the terms “polypeptide,” “oligopeptide,” “peptide,” and “protein” are used interchangeably herein to refer to amino acid polymers of any length. The polymer may be linear or branched, it may comprise modified amino acids, and it may be interrupted by non-amino acids. The terms also encompasses an amino acid polymer that has been modified naturally or by intervention, for example, by disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, D-amino acids, non-naturally occurring amino acids, and the like), as well as other modified amino acids known in the art.

General reference to “the composition” or “compositions” includes and is applicable to compositions of the invention.

A polypeptide or composition which is “isolated” is a polypeptide or composition which is in a form not found in nature. Isolated polypeptides or compositions include those which have been purified to a degree that they are no longer in a form in which they are found in nature. In some embodiments, a composition which is isolated is substantially pure.

As used herein, “substantially pure” refers to material that is at least 50% pure (i.e., free from contaminants), more preferably at least 90% pure, more preferably at least 95% pure, more preferably at least 98% pure, more preferably at least 99% pure.

As used herein, the singular form of the articles “a,” “an,” and “the” includes plural references unless indicated otherwise.

As used herein, the term “selectively binds to” refers to the specific binding of one protein to another (e.g., a receptor to an antigen), wherein the level of binding, as measured by any standard assay (e.g., an immunoassay), is statistically significantly higher than the background control for the assay. For example, when performing an immunoassay, controls typically include a reaction well or tube that contains antibody or an antigen-binding fragment alone (i.e., in the absence of antigen), wherein an amount of reactivity (e.g., non-specific binding to the well) by the antibody or antigen-binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, microcytometry, microarray, fluorescence activated cell sorting (FACS), flow cytometry, nuclear magnetic resonance (NMR) spectroscopy, and analytical ultracentrifugation.

Agents used in the methods of this invention can be randomly selected or rationally selected or designed. As used herein, in some embodiments, an agent is “randomly selected” when the agent is chosen randomly without considering the specific sequences involved in the association of CR2 and its ligands (e.g., C3d or EBV gp350). An example of randomly selected agents is the use of a phage display library.

“Carriers” as used herein include pharmaceutically acceptable carriers, excipients, or stabilizers used in pharmaceutical compositions or formulations that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™

The term “pharmaceutical formulation” refers to a preparation that is in such form as to permit the biological activity of the active ingredient to be effective, and that contains no additional components that are unacceptably toxic to a subject to which the formulation would be administered. Such formulations are generally sterile.

As used herein, the term an “effective amount” refers to at least an amount effective, at dosages and for periods of time necessary, to achieve the desired therapeutic or prophylactic result. As is understood by one skilled in the art, an effective amount of, for example, a CR2 ligand-specific non-antibody polypeptide of the present invention may vary, depending on, inter alia, patient history as well as other factors such as the type (and/or dosage) of the ligand-specific inhibitory non-antibody polypeptide used. An effective amount can be provided in one or more administrations. For purposes of this invention, an effective amount of a CR2 ligand-specific inhibitory non-antibody polypeptide is an amount sufficient to ameliorate, stabilize, reverse, slow, and/or delay progression of a condition associated with biological activity or interaction of CR2 and its ligands.

As used herein, the term “treatment” refers to an approach for obtaining beneficial or desired clinical results. For purposes of this invention, beneficial or desired clinical results include, but are not limited to, alleviation of one or more symptoms associated with a disease, diminishment of extent of disease, stabilizing (i.e., not worsening) one or more symptoms associated with the disease, delay or slowing of disease progression, amelioration or palliation of the disease state, and partial or total remission, whether detectable or undetectable. “Treatment” can also mean prolonging survival as compared to expected survival if not receiving treatment. In some embodiments, “treatment” of a disease can encompass curing a disease. In other embodiments, beneficial or desired results with respect to a condition include, but are not limited to, improving a condition, curing a condition, lessening severity of a condition, delaying progression of a condition, alleviating one or more symptoms associated with a condition, increasing the quality of life of one suffering from a condition, and/or prolonging survival.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual,” “subject,” or “patient” refers a mammal, including humans, domestic and farm animals, and zoo, sport, or pet animals, such as chimpanzees and other apes and monkey species, dogs, horses, rabbits, cattle, pigs, goats, sheep, hamsters, guinea pigs, gerbils, mice, ferrets, rats, cats, and the like. In some embodiments, the individual is human. The term does not denote a particular age or gender.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The term “IC50,” as used herein, refers to the concentration of an inhibitory peptide that is required for 50% inhibition of a CR2-ligand interaction (e.g., CR2-C3d or CR2-EBV gp350).

It is understood that whenever embodiments are described herein with the language “comprising,” otherwise analogous embodiments described in terms of “consisting of” and/or “consisting essentially of” are also provided.

Ligand-Specific Non-Antibody Polypeptides that Selectively Bind to CR2

In one aspect, provided herein are novel non-antibody polypeptides that selectively bind to a complement receptor type 2 (CR2) protein, a ligand thereof, or both, wherein the non-antibody polypeptides competitively inhibit binding of one or more CR2 ligands to a CR2 short consensus repeat (SCR) domain in a standard assay. In certain embodiments, the non-antibody polypeptides are novel CR2-ligand-specific non-antibody polypeptides that selectively bind to a CR2 protein, a ligand thereof, or both, wherein the non-antibody polypeptides competitively inhibit binding of one or more CR2 ligands to a CR2 SCR domain in a standard assay (e.g., a competitive ELISA). In some embodiments, the CR2 ligand is EBV (e.g., EBV gp350), a cell-bound proteolytic breakdown fragment of complement protein C3 (e.g., C3d, C3dg, and iC3b), IFN-a, or FcεRII/CD23.

As used herein, the term “standard assay” refers to assays commonly used to measure binding interactions between peptides, polypeptides, or proteins. Many such assays are known to those skilled in the art, including, for example, Western blots, immunoblots, enzyme-linked immunosorbant assays (ELISAs), including competitive ELISAs, radioimmunoassay (RIA), surface plasmon resonance, fluorescence activated cell sorting (FACS), flow cytometry, nuclear magnetic resonance (NMR) spectroscopy, and analytical ultracentrifugation. Standard assays also include, for example, competition assays to identify competitive inhibitors of particular ligands using standard techniques in the art (e.g., competitive ELISA or other binding assays).

A “competitive” inhibitor is an inhibitor (e.g., a non-antibody polypeptide, oligopeptide, peptide, nucleic acid, or carbohydrate, or other non-antibody compound as defined herein) that binds to a CR2 protein or to a ligand thereof and inhibits, reduces, or prevents binding of one or more of its ligands. For example, competitive inhibitors can be detected by their ability to inhibit the binding of a CR2-protein to any one or more of its natural ligands (e.g., EBV gp350, proteolytic fragments of complement protein C3, IFN-α, or FcεRII/CD23). A competitive inhibitor may bind to the target (e.g., a CR2 ligand, CR2 protein, or both) with a greater affinity for the target than an anti-CR2 antibody (e.g., mAb171). Competition assays in the presence of a ligand-selective non-antibody CR2-inhibitory polypeptide are described for example, in Examples 2 and 7, and Tables 1-2 and 4. Other competition assays include inhibition of CR2-induced B cell activation in vitro or in vivo, EBV binding and transformation, induction of IFN-a related mRNA changes, CD23-induced B cell class switching to IgE, increases in intracellular Calcium influx, and induction of tyrosine or serine/threonine phosphorylation.

As used herein, the term “complement receptor type 2,” “complement receptor 2,” “CR2,” “CD21,” “CR2 protein,” or “CR2/CD21,” refers to a transmembrane protein of ˜145 kilodaltons (“kD”) expressed on B cells, follicular dendritic cells, and some T cell subtypes or biologically-active fragments thereof (see, e.g., UniProtKB/Swiss-Prot Accession No. P20023). Hannan et al. (2002) Biochem. Soc. Trans. 30:983-989; Young et al. (2007) J. Biol. Chem. 282:36614-36625. CR2 is a member of the structural family of C3/C4 receptor and regulatory proteins known as the regulators of complement activation (“RCA”). Members of this family are characterized by the presence of short repeating domains of ˜70 amino acids known as short consensus repeat (“SCR”) modules. Each SCR contains a framework of highly conserved residues, including four cysteines, two prolines, one tryptophan, and several other partially conserved glycines and hydrophobic residue. The conserved cysteine residues form a pattern of disulfide bridges that connect Cys-1 to Cys-3 and Cys-2 to Cys-4. The modular composition of CR2 is well known, and consists of a 15- or 16-SCR extracellular domain, a 24-amino acid transmembrane domain, and a short 34-amino acid intracellular carboxyl-terminal tail. In human CR2 protein (SEQ ID NO:1), amino acids 1-20 comprise the leader peptide, amino acids 23-82 comprise SCR1 (SEQ ID NO: 2), amino acids 91-146 comprise SCR2 (SEQ ID NO: 3), amino acids 154-210 comprise SCR3, and amino acids 215-271 comprise SCR4. The segment (fragment) of human CR2 comprising both the SCR1 and SCR2 domains and a linker between SCR 1 and SCR2 is represented by SEQ ID NO:4.

The full-length mouse CR2 protein sequence is represented herein by SEQ ID NO:5 (see, e.g., UniProtKB/Swiss-Prot Accession No. P19070, which is incorporated herein by reference). In mouse CR2 protein (SEQ ID NO: 5), amino acids 14-73 comprise SCR1 and amino acids 82-138 comprise SCR2. The segment (fragment) of mouse CR2 that comprises both the SCR1 and SCR2 domains and the eight residue linker is located at positions 11-145 of SEQ ID NO: 5 and is represented by SEQ ID NO: 6. Human and mouse CR2 are approximately 66% identical over the full length amino acid sequences represented by SEQ ID NO: 1 and SEQ ID NO: 5 (using BLAST 2 pairwise alignment), and approximately 61% identical over the SR1-SCR2 domains of SEQ ID NO: 1 and SEQ ID NO: 5. It is understood that species and strain variations exist for the disclosed polypeptides, and proteins, and that the CR2 protein or biologically-active fragments thereof described herein encompass all species and strain variations.

The CR2 protein disclosed herein refers to a polypeptide that contains some or all of the ligand binding sites of the CR2 protein, and includes, but is not limited to, full-length CR2 proteins (e.g., human CR2 as shown in SEQ ID NO: 1 or mouse CR2 as shown in SEQ ID NO: 6), soluble CR2 proteins (e.g., a CR2 fragment comprising the complete extracellular domain of human CR2 protein), other biologically active fragments of CR2 proteins (e.g., a CR2 fragment comprising SCRs 1 and 2 of human CR2, or SCRs 1 to 8 of human CR2), CR2 fusion proteins, or any homologue of a naturally occurring CR2.

As used herein, the term “biologically-active fragments of CR2” refers to any fragment of CR2 capable of binding one or more CR2 ligands, such as cell-bound proteolytic fragments of complement protein C3 (i.e., iC3b, C3dg, and C3d), EBV gp350, CD23, and IFN-a. Such fragments include, for example, the complete extracellular domain of human or mouse CR2, a fragment comprising SCRs 1 to 8 of human or mouse CR2, or a fragment comprising SCRs 1 to 2 of human or mouse CR2. All known CR2 ligands bind within the first two amino-terminal SCR domains (SCR 1 and SCR 2) at overlapping but distinguishable binding sites, although an additional glycosylation-dependent interaction with CD23 also involves SCRs 3 to 5. See Young et al., J. Biol. Chem. (2007) 282(50):36614-36625.

In some embodiments, the CR2 protein comprises at least the first two N-terminal SCR domains of a human CR2 protein, such as a CR2 portion having an amino acid sequence containing at least amino acids 23 through 146 of the human CR2 protein. In other embodiments, the CR2 protein comprises at least the first two SCR domains of human CR2 protein having an amino acid sequence that is at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to amino acids 23 through 146 of the full-length human CR2 protein (SEQ ID NO:1).

A homologue of a CR2 protein or a fragment thereof includes proteins which differ from a naturally occurring CR2 (or a biologically-active fragment of a CR2 protein) in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). In some embodiments, a CR2 homologue has an amino acid sequence that is at least about 70% identical to the amino acid sequence of a naturally occurring CR2 protein (e.g., SEQ ID NO: 1 or SEQ ID NO: 5), for example at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring CR2 protein (e.g., SEQ ID NO: 1 or SEQ ID NO: 5). A CR2 homologue or a biologically-active fragment preferably retains the ability to bind to a naturally occurring ligand of CR2 (e.g., C3d (including any C3 fragments with CR2-binding ability), FcεRII/CD23, EBV gp320, and cytokine interferon alpha (IFN-α)). Such homologues include fragments of a full length CR2 (e.g., the SCR2 domain or the SCR1-SCR2 domain) and can be referred to herein as a CR2 ligand-binding fragment. In one embodiment, a CR2 homologue has the biological activity of a naturally occurring CR2. Reference to a CR2 protein can also generally refer to CR2 in complex with a ligand.

As used herein, “percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways, for example, using publicly available computer software such as BLAST, BLAST-2, ALIGN or MEGALIGN™ (DNASTAR) software. One skilled in the art can determine appropriate parameters for measuring alignment, including any algorithms needed to achieve maximal alignment over the full length of the sequences being compared.

As used herein, the term “CR2 ligand” refers to natural ligands capable of binding to the extracellular domain of CR2. Natural ligands for CR2 include, but are not limited to, iC3b, C3dg, C3d (cell-bound breakdown fragments of complement protein C3 that bind to the two N-terminal SCR domains of CR2 (SCR 1 and 2)). Iida et al. (1983) J. Exp. Med. 158:1021-1033. Cleavage of C3 results initially in the generation of C3b and the covalent attachment of this C3b to the activating cell surface. The C3b fragment is involved in the generation of enzymatic complexes that amplify the complement cascade. On a cell surface, C3b is rapidly converted to inactive iC3b, particularly when deposited on a host surface containing regulators of complement activation (i.e., most host tissue). Even in the absence of membrane-bound complement regulators, substantial levels of iC3b are formed. iC3b is subsequently digested to the membrane bound fragments C3dg and then C3d by serum proteases, but this process is relatively slow. Thus, the C3 ligands for CR2 are relatively long lived once they are generated and are present in high concentrations at sites of complement activation. CR2 therefore can serve as a potent targeting vehicle for bringing molecules to sites of complement activation.

Other CR2 ligands include, but are not limited to the Epstein-Barr Virus 350 kilodalton surface glycoprotein (“EBV gp350”) or its truncated 220 kilodalton form (“EBV gp220”), collectively referred to as “EBV gp350/220” (Young et al. (2007), J. Biol. Chem. 282:36614-36625), interferon alpha (“IFN-α”) (Asokan et al. (2006) J Immunol: 177:383-394), and CD23 (Aubry et al. (1992) Nature 358:505-507; and Aubry et al. (1994) J. Immunol. 152:5806-5813). Also known as FcεRII, CD23 is a low affinity receptor for immunoglobulin E (“IgE”), an antibody isotype involved in allergy and resistance to parasites. Unlike many of the antibody Fc receptors, FcεRII/CD23 is a C-type lectin found on mature B cells, activated macrophages, eosinophils, follicular dendritic cells and platelets.

Epstein-Barr virus (EBV/HHV4) is a human gamma-herpesvirus that primarily infects two main cell types, B-lymphocytes and epithelial cells. Under some circumstances the virus can also infect T-cells, natural killer cells, smooth muscle and astrocyte. See Menet et al. (1999) J. Virol. 73:7722-33; Rickinson and Kieff, D. M. Knipe and P. M. Howley (ed.), Fields Virology, Lippinocott Williams & Wilkins, Philadelphia, Pa., pp. 2655-2700. In B-cells, the interaction between EBV and the cell surface is initially mediated by the predominant glycoprotein in the viral envelope, EBV gp350, binding to complement receptor type 2 (CR2/CD21) Fingeroth et al. (1984) Proc. Nat'l Acad. Sci. USA 81:4510-4; Frade et al. (1985) Proc. Nat'l Acad. Sci. USA 82:1490-3; Johannsen et al. (2004) Proc. Nat'l Acad. Sci. USA 101:16286-91; Nemerow et al. (1987) J. Virol. 61:1416-20; Nemerow et al. (1985) J. Virol. 55:347-51; and Tanner et al. (1988) J. Virol. 62:4452-64. Fusion with the cell membrane and invasion of the host cell is facilitated by the binding of a second viral glycoprotein, gp42, to human leukocyte antigen (HLA) class II molecules and also by three additional viral glycoproteins, gB, gH and gL. See Hutt-Fletcher (2007) J. Virol. 81:7825-32; Haan et al. (2001) Virology 290:106-14; Haddad et al. (1989) J. Virol. 63:4998-5005; Molesworth et al. (2000) J. Virol. 74:6324-32. While recombinant EBV gp350-deletion strains are capable of transforming human B-lymphocytes, they do so with a significantly reduced efficiency. Janz et al. (2000) J Virol 74:10142-52.

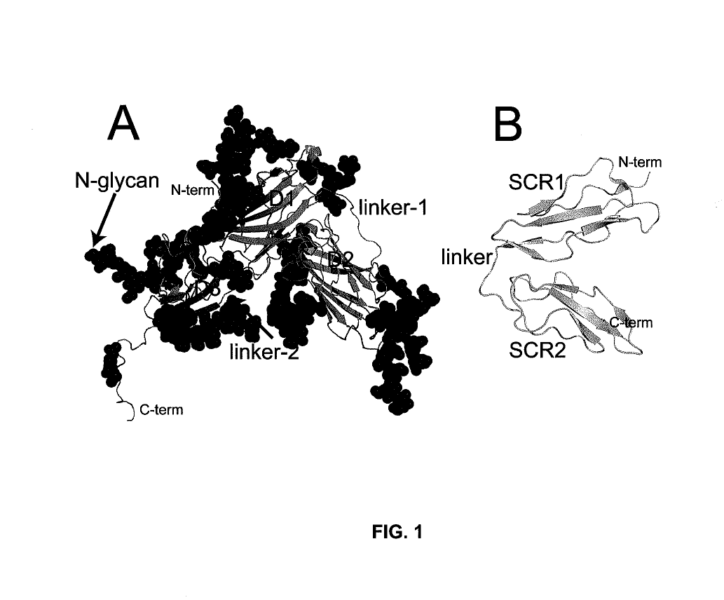

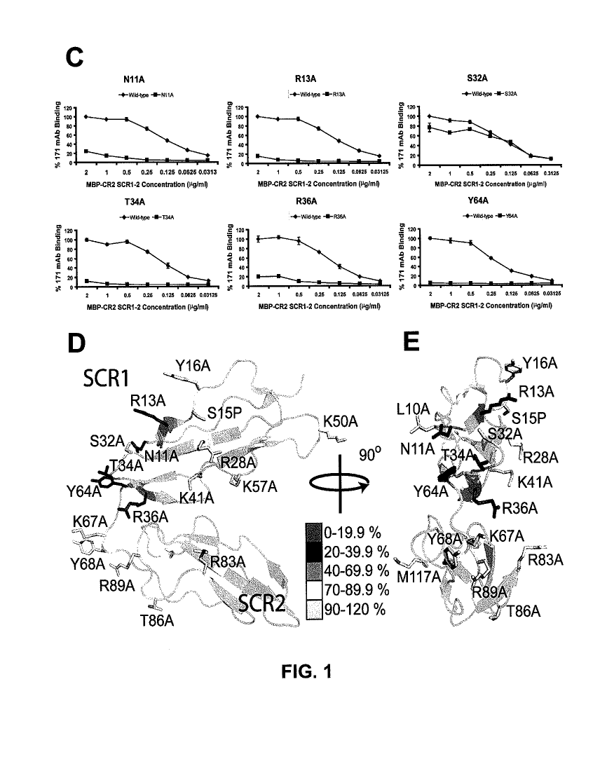

The crystal structure of a truncated form of EBV gp350 comprising the amino-terminal 470 residues identified three distinct domains of the EBV gp350 residues: 4-153, 165-305, and 317-426. See Szakonyi et al. (2006) Nat. Struct. Mol. Biol. 13:996-1001. Each residue comprises an anti-parallel beta-barrel structure, and is joined by two linker sequences, each of eleven amino acid residues. These domains are packed tightly against each other forming a distinctive ‘L’-shaped arrangement that is almost uniformly glycosylated (FIG. 1A). The putative CR2-binding site of EBV gp350 within a negatively charged region of this molecule incorporates the two N-terminal domains and the linker region connecting them (residues 154-164). See id. Site-directed mutagenesis studies indicate that a surface devoid of carbohydrate moieties is the region required for the CR2-EBV gp350 interaction. Szakonyi et al. (2006) Nat. Struct. Mol. Biol. 13:996-1001. Those mutations affecting CR2 binding also disrupted the ability of EBV gp350 to be recognized by its major neutralizing monoclonal antibody, 72A1. They are in close proximity to, or directly overlap with the CR2-binding domain identified from the mutagenesis and crystal studies.

EBV is found in a large proportion (>90%) of the world's population. While childhood EBV infection is often clinically silent, delayed infection until adolescence or later can result in the development of infectious mononucleosis. In immunodeficient individuals, EBV has been linked to malignancies or disease states, including, but not limited to, B-cell lymphomas, African Burkitt's lymphoma, nasopharyngeal carcinoma, EBV-associated lymphoproliferative disease, non-Hodgkin's lymphoma, oral hairy leukoplakia, and human immunodeficiency virus-1 (HIV-1) related lymphoproliferative disorders. In addition, EBV has been associated with autoimmune diseases, including but not limited to systemic lupus erythematosus, rheumatoid arthritis, and multiple sclerosis. Finally, ongoing EBV infection has been linked to chronic fatigue syndrome. See Murray et al., in EPSTEIN-BARR VIRUS (2005) (E. S. Robertson (ed.) Caister Academic Press, Wymondham, England), pp. 93-111; and Cohen et al., in EPSTEIN-BARR VIRUS (2005) (E. S. Robertson (ed.) Caister Academic Press, Wymondham, England), pp. 35-54.

In some embodiments, the present invention provides non-antibody polypeptides that selectively bind to a complement receptor type 2 (CR2) protein, a ligand thereof, or both, wherein the non-antibody polypeptides competitively inhibit ligand binding to CR2 protein in a standard assay (e.g., a competitive ELISA). In some embodiments, the CR2 ligand is Epstein-Barr Virus (EBV), and the non-antibody polypeptides competitively inhibit EBV binding to CR2 protein in a standard assay. In some embodiments, the CR2 ligand is EBV gp350, and the non-antibody polypeptides competitively inhibit EBV binding to CR2 protein in a standard assay. In some embodiments, the non-antibody polypeptides competitively inhibit EBV gp350 binding to a CR2 SCR domain in a standard assay. In some embodiments, the CR2 SCR domains are SCRs 1 and 2. In any of the embodiments described herein, the standard assay is a competitive ELISA.

In some embodiments, the non-antibody polypeptides selectively bind to EBV and competitively inhibit EBV binding to CR2 protein in a standard assay. In some embodiments, the non-antibody polypeptides selectively bind to EBV gp350 and competitively inhibit EBV binding to CR2 protein in a standard assay. In some embodiments, the non-antibody polypeptides selectively bind to a binding site on EBV gp350 comprising two N-terminal β-barrel domains (D1 and D2) (see, e.g., FIG. 1) and a linker sequence between D1 and D2 (linker-1) and competitively inhibit EBV binding to CR2 protein in a standard assay. In some embodiments, the non-antibody polypeptides selectively bind to a binding site on EBV gp350 comprising D1 and D2 (see, e.g., FIG. 1) and competitively inhibit EBV binding to CR2 protein in a standard assay. In some embodiments, the standard assay is a competitive ELISA.

In some embodiments, the non-antibody polypeptides selectively bind to a binding site on EBV gp350 comprising amino acid positions E21, D22, E155, E201, D208, E210, and D296 in a wildtype EBV gp350 sequence or their equivalent positions in a variant EBV gp350 sequence, and competitively inhibit EBV binding to CR2 protein in a standard assay. In some embodiments, the non-antibody polypeptides selectively bind to a binding site on EBV gp350 comprising amino acid positions Y151, I160, and W162 in a wildtype EBV gp350 sequence or their equivalent positions in a variant EBV gp350 sequence and competitively inhibit EBV binding to CR2 protein in a standard assay. In some embodiments, the non-antibody polypeptides selectively bind to a binding site on EBV gp350 comprising amino acid positions E21, D22, E155, E201, D208, E210, and D296, Y151, I160, and W162 in a wildtype EBV gp350 sequence or their equivalent positions in a variant EBV gp350 sequence and competitively inhibit EBV binding to CR2 protein in a standard assay. In any of the embodiments described herein, the standard assay is a competitive ELISA.

In some embodiments, any of the non-antibody polypeptides that selectively bind EBV gp350 and competitively inhibit EBV binding to CR2 protein in a standard assay described herein bind EBV gp350 at an IC50 of about 700 μM or less, about 640 μM or less, about 600 μM or less, about 450 μM or less, about 190 μM or less, about 150 μM or less, about 100 μM or less, about 95 μM or less, about 70 μM or less, about 40 μM or less, about 20 μM or less, or about 10 μM or less. In any of the embodiments described herein, the standard assay is a competitive ELISA. In some embodiments, any of the non-antibody polypeptides that selectively bind EBV gp350 and competitively inhibit EBV binding to CR2 protein in a standard assay described herein bind EBV gp350 at an IC50 between about 10 μM and about 700 μM, between about 100 μM and about 700 μM, between about 250 μM and about 700 μM, between about 500 μM and about 700 μM, between about 10 μM and about 600 μM, between about 100 μM and about 600 μM, between about 250 μM and about 600 μM, between about 400 μM and about 600 μM, between about 10 μM and about 450 μM, between about 50 μM and about 450 μM, between about 100 μM and about 450 μM, between about 250 μM and about 450 μM, between about 350 μM and about 450 μM, between about 10 μM and about 190 μM, between about 50 μM and about 190 μM, between about 100 μM and about 190 μM, between about 10 μM and about 150 μM, between about 50 μM and about 150 μM, or between about 10 μM and about 100 μM.

In some embodiments, the non-antibody polypeptides selectively bind to CR2 protein and competitively inhibit EBV binding to CR2 protein in a standard assay. In some embodiments, the non-antibody polypeptides selectively bind to the extracellular domain of CR2 protein and competitively inhibit EBV binding to CR2 protein in a standard assay. In some embodiments, the non-antibody polypeptides selectively bind to SCRs 1 to 8 of the extracellular domain of CR2 protein and competitively inhibit EBV binding to CR2 protein in a standard assay. In some embodiments, the non-antibody polypeptides selectively bind to SCRs 1 and 2 of CR2 protein, including the linker between SCRs 1 and 2 and competitively inhibit EBV binding to CR2 protein in a standard assay. In some embodiments, the non-antibody polypeptides selectively bind to SCRs 1 and 2 of the extracellular domain of CR2 protein and competitively inhibit EBV binding to CR2 protein in a standard assay. In some embodiments, the non-antibody polypeptides selectively bind to SCR 1 of the extracellular domain of CR2 protein and competitively inhibit EBV binding to CR2 protein in a standard assay. In some embodiments, the non-antibody polypeptides selectively bind to SCR 2 of the extracellular domain of CR2 protein and competitively inhibit EBV binding to CR2 protein in a standard assay. In some embodiments, the non-antibody polypeptides selectively bind to an SCR domain of a biologically-active fragment of CR2 protein and competitively inhibit EBV binding to CR2 protein in a standard assay. In some embodiments, the biologically-active fragment of CR2 protein comprises SCRs 1 and 2. In any of the embodiments described herein, the standard assay is a competitive ELISA.

In some embodiments, the non-antibody polypeptides selectively bind to a binding site on CR2 protein comprising amino acid positions R13, S15, A22, R28, F35, R36, K41, D49, and D52 in a human CR2 protein sequence or their equivalent positions in a non-human CR2 protein sequence, and competitively inhibit EBV binding to CR2 protein in a standard assay. In some embodiments, the non-antibody polypeptides selectively bind to a binding site on CR2 protein comprising amino acid positions R13, A22, R28, F35, R36, D49, K41, D52, K57, E63, K67, Y68, R83, and R89 in a human CR2 protein sequence or their equivalent positions in a non-human CR2 protein sequence, and competitively inhibit EBV binding to CR2 protein in a standard assay. In some embodiments, the non-antibody polypeptides selectively bind to a binding site on CR2 protein comprising amino acid positions R13, A22, R28, K67, Y68 R83, G84, R89 and S103 in a human CR2 protein sequence or their equivalent positions in a non-human CR2 protein sequence, and competitively inhibit EBV binding to CR2 protein in a standard assay. In any of the embodiments described herein, the standard assay is a competitive ELISA.

In some embodiments, the non-antibody polypeptide selectively binds CR2 protein, competitively inhibits EBV binding to CR2 protein in a standard assay and comprises the amino acid sequence CSEGSLKGC (SEQ ID NO: 20). In some embodiments, the non-antibody polypeptide selectively binds to the extracellular domain of CR2 protein, competitively inhibits EBV binding to CR2 protein in a standard assay and comprises the amino acid sequence CSEGSLKGC (SEQ ID NO: 20). In some embodiments, the non-antibody polypeptide selectively binds to SCRs 1 to 8 of the extracellular domain of CR2 protein, competitively inhibits EBV binding to CR2 protein in a standard assay, and comprises the amino acid sequence CSEGSLKGC (SEQ ID NO: 20). In some embodiments, the non-antibody polypeptide selectively binds to SCRs 1 and 2 of CR2 protein, including the linker between SCRs 1 and 2, competitively inhibits EBV binding to CR2 protein in a standard assay and comprises the amino acid sequence CSEGSLKGC (SEQ ID NO: 20). In some embodiments, the non-antibody polypeptide selectively binds to SCRs 1 and 2 of the extracellular domain of CR2 protein, competitively inhibits EBV binding to CR2 protein in a standard assay and comprises the amino acid sequence CSEGSLKGC (SEQ ID NO: 20). In some embodiments, the non-antibody polypeptide selectively binds to SCR 1 of the extracellular domain of CR2 protein, competitively inhibits EBV binding to CR2 protein in a standard assay and comprises the amino acid sequence CSEGSLKGC (SEQ ID NO: 20). In some embodiments, the non-antibody polypeptide selectively binds to SCR 2 of the extracellular domain of CR2 protein, competitively inhibits EBV binding to CR2 protein in a standard assay and comprises the amino acid sequence CSEGSLKGC (SEQ ID NO: 20). In some embodiments, the non-antibody polypeptide selectively binds to an SCR domain of a biologically-active fragment of CR2 protein, competitively inhibits EBV binding to CR2 protein in a standard assay and comprises the amino acid sequence CSEGSLKGC (SEQ ID NO: 20). In some embodiments, the biologically-active fragment of CR2 protein comprises SCRs 1 and 2. In some embodiments, the non-antibody polypeptide selectively binds to CR2 protein, comprises the amino acid sequence CSEGSLKGC (SEQ ID NO: 20), selectively binds to a binding site on CR2 protein comprising amino acid positions R13, A22, R28, K67, Y68 R83, G84, R89 and S103 in a human CR2 protein sequence or their equivalent positions in a non-human CR2 protein sequence and competitively inhibits EBV binding to CR2 protein in a standard assay. In any of the embodiments described herein, the standard assay is a competitive ELISA.

In some embodiments, any of the non-antibody polypeptides that selectively bind CR2 protein and competitively inhibit EBV binding to CR2 protein in a standard assay described herein bind CR2 protein at an IC50 of about 700 μM or less, about 640 μM or less, about 600 μM or less, about 450 μM or less, about 190 μM or less, about 150 μM or less, about 100 μM or less, about 95 μM or less, about 70 μM or less, about 40 μM or less, about 20 μM or less, or about 10 μM or less. In any of the embodiments described herein, the standard assay is a competitive ELISA. In some embodiments, any of the non-antibody polypeptides that selectively bind CR2 protein and competitively inhibit EBV binding to CR2 protein in a standard assay described herein bind CR2 protein at an IC50 between about 10 μM and about 700 μM, between about 100 μM and about 700 μM, between about 250 μM and about 700 μM, between about 500 μM and about 700 μM, between about 10 μM and about 600 μM, between about 100 μM and about 600 μM, between about 250 μM and about 600 μM, between about 400 μM and about 600 μM, between about 10 μM and about 450 μM, between about 50 μM and about 450 μM, between about 100 μM and about 450 μM, between about 250 μM and about 450 μM, between about 350 μM and about 450 μM, between about 10 μM and about 190 μM, between about 50 μM and about 190 μM, between about 100 μM and about 190 μM, between about 10 μM and about 150 μM, between about 50 μM and about 150 μM, or between about 10 μM and about 100 μM.

In some embodiments, the non-antibody polypeptides selectively bind to both a ligand of CR2 protein (e.g., EBV gp350, proteolytic fragments of complement protein C3, IFN-α, or FcεRII/CD23) and CR2 protein, wherein the non-antibody polypeptides competitively inhibit CR2 ligand binding to CR2 protein in a standard assay. In some embodiments, the CR2 ligand is EBV and the non-antibody polypeptides competitively inhibit EBV binding to CR2 protein in a standard assay. In some embodiments, the CR2 ligand is EBV gp350 and the non-antibody polypeptides competitively inhibit EBV binding to CR2 protein in a standard assay. In some embodiments, the non-antibody polypeptides competitively inhibit EBV binding to one or more SCR domains of CR2 protein. In some embodiments, the one or more SCR domains of CR2 protein comprises the extracellular domain of CR2 protein. In some embodiments, the one or more SCR domains of CR2 protein comprises SCRs 1 to 8 of the extracellular domain of CR2 protein. In some embodiments, the one or more SCR domains of CR2 protein comprises SCRs 1 and 2 of CR2 protein, including the linker between SCRs 1 and 2. In some embodiments, the one or more SCR domains of CR2 protein comprises SCRs 1 and 2 of the extracellular domain of CR2 protein. In some embodiments, the one or more SCR domains of CR2 protein comprises SCR 1 of the extracellular domain of CR2 protein. In some embodiments, the one or more SCR domains of CR2 protein comprises SCR 2 of the extracellular domain of CR2 protein. In some embodiments, the non-antibody polypeptides selectively bind to one or more SCR domains of a biologically-active fragment of CR2 protein and competitively inhibit EBV binding to CR2 protein in a standard assay. In some embodiments, the one or more SCR domains of a biologically-active fragment of CR2 protein comprise SCRs 1 and 2. In any of the embodiments described herein, the standard assay is a competitive ELISA.

The invention also provides non-antibody polypeptides that selectively bind to a CR2 protein, a ligand thereof, or both, wherein the ligand is a cell-bound proteolytic fragment of complement protein C3 (“C3 fragment”), and wherein the non-antibody polypeptide competitively inhibits the binding of one or more C3 fragments to CR2 protein in a standard assay. In some embodiments, the non-antibody polypeptides selectively bind to one or more C3 fragments and competitively inhibit the binding of one or more C3 fragments to CR2 protein in a standard assay. In some embodiments, the C3 fragments include, but are not limited to, C3d, C3dg, and iC3b. In some embodiments, the non-antibody polypeptides selectively bind to C3d and competitively inhibit the binding of C3d to CR2 protein in a standard assay. In any of the above embodiments, the standard assay is a competitive ELISA.

In some embodiments, the non-antibody polypeptides selectively bind to CR2 protein and competitively inhibit the binding of one or more C3 fragments to CR2 protein in a standard assay. In some embodiments, the C3 fragments include, but are not limited to, C3d, C3dg, and iC3b. In some embodiments, the non-antibody polypeptides selectively bind to a site on CR2 protein comprising SCRs 1 and 2 and the inter-SCR linker between SCRs 1 and 2 (SEQ ID NO: 4) of CR2 protein and competitively inhibit binding of one or more C3 fragments to CR2 protein in a standard assay. In some embodiments, the non-antibody polypeptides selectively bind to a site on CR2 protein comprising SCRs 1 and 2 and the inter-SCR linker between SCRs 1 and 2 (SEQ ID NO: 4) of CR2 protein and competitively inhibit binding of C3d to CR2 protein in a standard assay. In some embodiments, the non-antibody polypeptides selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, and competitively inhibit binding of one or more C3 fragments to CR2 protein in a standard assay. In some embodiments, the non-antibody polypeptides selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, and competitively inhibit binding of C3d to CR2 protein in a standard assay. In any of the embodiments described herein, the standard assay is a competitive ELISA. In some embodiments, the non-antibody polypeptides selectively bind to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence, and competitively inhibit binding of one or more C3 fragments to CR2 protein in a standard assay. In some embodiments, the non-antibody polypeptides selectively bind to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence, and competitively inhibit binding of C3d to CR2 protein in a standard assay. In any of the embodiments described herein, the standard assay is a competitive ELISA

In some embodiments, the non-antibody polypeptides that selectively bind CR2 protein and competitively inhibit binding of one or more C3 fragments to CR2 protein in a standard assay comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13). In some embodiments, the one or more C3 fragments include, but are not limited to, C3d, C3dg, and iC3b. In some embodiments, the non-antibody polypeptides that selectively bind CR2 protein and competitively inhibit binding of one or more C3 fragments to CR2 protein competitively inhibit binding of C3d to CR2 protein in a standard assay. In any of the embodiments described herein, the standard assay is a competitive ELISA.

In some embodiments, the non-antibody polypeptides selectively bind to a site on CR2 protein comprising SCRs 1 and 2 and the inter-SCR linker between SCRs 1 and 2 (SEQ ID NO: 4) of CR2 protein, competitively inhibit binding of one or more C3 fragments to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13). In some embodiments, the one or more C3 fragments include, but are not limited to, C3d, C3dg, and iC3b. In some embodiments, the non-antibody polypeptides that selectively bind CR2 protein at said site on CR2 protein competitively inhibit binding of C3d to CR2 protein in a standard assay. In any of the embodiments described herein, the standard assay is a competitive ELISA.

In some embodiments, the non-antibody polypeptides selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, competitively inhibit binding of one or more C3 fragments to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13). In some embodiments, the one or more C3 fragments include, but are not limited to, C3d, C3dg, and iC3b. In some embodiments, the non-antibody polypeptide that selectively binds CR2 protein at said site on CR2 protein competitively inhibits binding of C3d to CR2 protein in a standard assay.

In some embodiments, the non-antibody polypeptide that selectively binds CR2 protein and competitively inhibits binding of one or more C3 fragments to CR2 protein in a standard assay comprises an amino acid sequence APQHLSSQYSRT (SEQ ID NO: 14). In some embodiments, the non-antibody polypeptide that selectively binds CR2 protein and competitively inhibits binding of one or more C3 fragments to CR2 protein in a standard assay comprises an amino acid sequence APQALSSQYSRT (SEQ ID NO: 15). In some embodiments, the non-antibody polypeptide that selectively binds CR2 protein and competitively inhibits binding of one or more C3 fragments to CR2 protein in a standard assay comprises an amino acid sequence APQHLSSQ (SEQ ID NO: 16). In some embodiments, the non-antibody polypeptide that selectively binds CR2 protein and competitively inhibits binding of one or more C3 fragments to CR2 protein in a standard assay comprises an amino acid sequence APAHLSSQ (SEQ ID NO: 17). In some embodiments, the non-antibody polypeptide that selectively binds CR2 protein and competitively inhibits binding of one or more C3 fragments to CR2 protein in a standard assay comprises an amino acid sequence ISTSNPRHHSTA (SEQ ID NO: 18). In some embodiments, the non-antibody polypeptide that selectively binds CR2 protein and competitively inhibits binding of one or more C3 fragments to CR2 protein in a standard assay comprises an amino acid sequence ISTSNPRAASTA (SEQ ID NO: 19). In some embodiments, the one or more C3 fragments include, but are not limited to, C3d, C3dg, and iC3b. In some embodiments, the non-antibody polypeptide that selectively binds CR2 protein and competitively inhibits binding of one or more C3 fragments to CR2 protein in a standard assay competitively inhibits binding of C3d to CR2 protein in a standard assay. In any of the above embodiments, the standard assay is a competitive ELISA.

In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay comprises an amino acid sequence APQHLSSQYSRT (SEQ ID NO: 14). In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay comprises an amino acid sequence APQALSSQYSRT (SEQ ID NO: 15). In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay comprises an amino acid sequence APQHLSSQ (SEQ ID NO: 16). In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay comprises an amino acid sequence APAHLSSQ (SEQ ID NO: 17). In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay comprises an amino acid sequence ISTSNPRHHSTA (SEQ ID NO: 18). In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay comprises an amino acid sequence ISTSNPRAASTA (SEQ ID NO: 19). In some embodiments, the one or more C3 fragments include, but are not limited to, C3d, C3dg, and iC3b. In some embodiments, the non-antibody polypeptide that selectively binds to a site on CR2 protein comprising SCRs 1 and 2 and the inter-SCR linker between SCRs 1 and 2 (SEQ ID NO: 4) of CR2 protein and competitively inhibits binding of one or more C3 fragments to CR2 protein in a standard assay competitively inhibits binding of C3d to CR2 protein in a standard assay. In any of the above embodiments, the standard assay is a competitive ELISA.

In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay comprises an amino acid sequence APQHLSSQYSRT (SEQ ID NO: 14). In some embodiments, the non-antibody polypeptide that selectively binds to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence and competitively inhibits binding of one or more C3 fragments to CR2 protein in a standard assay comprises an amino acid sequence APQHLSSQYSRT (SEQ ID NO: 14). In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay comprises an amino acid sequence APQALSSQYSRT (SEQ ID NO: 15). In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay comprises an amino acid sequence APQHLSSQ (SEQ ID NO: 16). In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay comprises an amino acid sequence APAHLSSQ (SEQ ID NO: 17). In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay comprises an amino acid sequence ISTSNPRHHSTA (SEQ ID NO: 18). In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay comprises an amino acid sequence ISTSNPRAASTA (SEQ ID NO: 19). In some embodiments, the one or more C3 fragments include, but are not limited to, C3d, C3dg, and iC3b. In some embodiments, the non-antibody polypeptide that selectively binds to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence and competitively inhibits binding of one or more C3 fragments to CR2 protein in a standard assay competitively inhibits binding of C3d to CR2 protein in a standard assay. In some embodiments, the non-antibody polypeptide that selectively binds to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence and competitively inhibits binding of one or more C3 fragments to CR2 protein in a standard assay competitively inhibits binding of C3d to CR2 protein in a standard assay. In any of the above embodiments, the standard assay is a competitive ELISA.

In some embodiments, any of the non-antibody polypeptides that selectively bind CR2 protein and competitively inhibit binding of one or more C3 fragments to CR2 protein in a standard assay described herein bind CR2 at an IC50 of about 700 μM or less, about 640 μM or less, about 600 μM or less, about 450 μM or less, about 190 μM or less, about 150 μM or less, about 100 μM or less, about 95 μM or less, about 70 μM or less, about 40 μM or less, about 20 μM or less, or about 10 μM or less. In any of the embodiments described herein, the standard assay is a competitive ELISA. In some embodiments, any of the non-antibody polypeptides that selectively bind CR2 protein and competitively inhibit binding of one or more C3 fragments to CR2 protein in a standard assay described herein bind CR2 at an IC50 between about 10 μM and about 700 μM, between about 100 μM and about 700 μM, between about 250 μM and about 700 μM, between about 500 μM and about 700 μM, between about 10 μM and about 600 μM, between about 100 μM and about 600 μM, between about 250 μM and about 600 μM, between about 400 μM and about 600 μM, between about 10 μM and about 450 μM, between about 50 μM and about 450 μM, between about 100 μM and about 450 μM, between about 250 μM and about 450 μM, between about 350 μM and about 450 μM, between about 10 μM and about 190 μM, between about 50 μM and about 190 μM, between about 100 μM and about 190 μM, between about 10 μM and about 150 μM, between about 50 μM and about 150 μM, or between about 10 μM and about 100 μM.

In some embodiments, the non-antibody polypeptides selectively bind to both a ligand of CR2 protein (e.g., EBV gp350, proteolytic fragments of complement protein C3, IFN-α, or CD23) and CR2 protein, wherein the non-antibody polypeptides competitively inhibit CR2 ligand binding to CR2 protein in a standard assay. In some embodiments, the CR2 ligand is one or more C3 fragments and the non-antibody polypeptides competitively inhibit C3 fragment binding to CR2 protein in a standard assay. In some embodiments, the C3 fragments comprise C3d, C3dg, and iC3b. In some embodiments, the CR2 ligand is C3d and the non-antibody polypeptides competitively inhibit C3d binding to CR2 protein in a standard assay. In some embodiments, the non-antibody polypeptides competitively inhibit C3d binding to one or more SCR domains of CR2 protein. In some embodiments, the one or more SCR domains of CR2 protein comprises the extracellular domain of CR2 protein. In some embodiments, the one or more SCR domains of CR2 protein comprises SCRs 1 to 8 of the extracellular domain of CR2 protein. In some embodiments, the one or more SCR domains of CR2 protein comprises SCRs 1 and 2 of CR2 protein, including the linker between SCRs 1 and 2. In some embodiments, the one or more SCR domains of CR2 protein comprises SCRs 1 and 2 of the extracellular domain of CR2 protein. In some embodiments, the one or more SCR domains of CR2 protein comprises SCR 1 of the extracellular domain of CR2 protein. In some embodiments, the one or more SCR domains of CR2 protein comprises SCR 2 of the extracellular domain of CR2 protein. In some embodiments, the non-antibody polypeptides selectively bind to one or more SCR domains of a biologically-active fragment of CR2 protein and competitively inhibit EBV binding to CR2 protein in a standard assay. In some embodiments, the one or more SCR domains of a biologically-active fragment of CR2 protein comprise SCRs 1 and 2. In any of the embodiments described herein, the standard assay is a competitive ELISA.

In some embodiments, the present invention provides non-antibody polypeptides that selectively bind to a complement receptor type 2 (CR2) protein, a ligand thereof, or both, wherein the non-antibody polypeptides competitively inhibit ligand binding to CR2 protein in a standard assay (e.g., a competitive ELISA). In some embodiments, the CR2 ligand is IFN-α. In some embodiments, the non-antibody polypeptides bind to IFN-α and competitively inhibit IFN-α-binding to CR2 protein in a standard assay. In some embodiments, the non-antibody polypeptides bind to CR2 protein and competitively inhibit IFN-α-binding to CR2 protein in a standard assay. In some embodiments, the non-antibody polypeptides bind to a site in SCRs 1 and 2 of CR2 protein and competitively inhibit IFN-α-binding to CR2 protein in a standard assay. In any of the embodiments described herein, the standard assay is a competitive ELISA.

In some embodiments, the present invention provides non-antibody polypeptides that selectively bind to a complement receptor type 2 (CR2) protein, a ligand thereof, or both, wherein the non-antibody polypeptides competitively inhibit ligand binding to CR2 protein in a standard assay (e.g., a competitive ELISA). In some embodiments, the CR2 ligand is FcεRII/CD23. In some embodiments, the non-antibody polypeptides bind to FcεRII/CD23 and competitively inhibit FcεRII/CD23-binding to CR2 protein in a standard assay. In some embodiments, the non-antibody polypeptides bind to CR2-protein and competitively inhibit FcεRII/CD23-binding to CR2 protein in a standard assay. In some embodiments, the non-antibody polypeptides bind to a site in SCRs 1 and 2 of CR2 protein and competitively inhibit FcεRII/CD23-binding to CR2 protein in a standard assay. In any of the embodiments described herein, the standard assay is a competitive ELISA.

### Pharmaceutical Formulations

Provided herein are pharmaceutical compositions and kits comprising any one or more of the non-antibody polypeptides described herein and a pharmaceutically acceptable carrier suitable for various routes of administration. Also provided herein are kits comprising any one or more of the non-antibody polypeptides described herein a pharmaceutically acceptable carrier suitable for various routes of administration, and optionally instructions for use of the non-antibody polypeptides with any of the methods described herein.

The pharmaceutical formulations may be suitable for a variety of modes of administration as described herein, including, for example, systemic or localized administration. The pharmaceutical formulations described herein can be in the form of injectable solutions. The pharmaceutical formulations described herein can be packaged in single unit dosages or in multidosage forms.

In some embodiments, the pharmaceutical compositions comprise any one or more non-antibody polypeptides that selectively bind to a complement receptor type 2 (CR2) protein, a ligand thereof, or both, wherein the non-antibody polypeptides competitively inhibit ligand binding to CR2 protein in a standard assay (e.g., a competitive ELISA) and a pharmaceutically acceptable carrier suitable for administration to an individual. In some embodiments, the pharmaceutical compositions comprise any one or more non-antibody polypeptides that selectively bind to a complement receptor type 2 (CR2) protein, a ligand thereof, or both, wherein the non-antibody polypeptides competitively inhibit ligand binding to CR2 protein in a standard assay (e.g., a competitive ELISA) and a pharmaceutically acceptable carrier suitable for oral administration to an individual. In some embodiments, the pharmaceutical compositions comprise any one or more non-antibody polypeptides that selectively bind to a complement receptor type 2 (CR2) protein, a ligand thereof, or both, wherein the non-antibody polypeptides competitively inhibit ligand binding to CR2 protein in a standard assay (e.g., a competitive ELISA) and a pharmaceutically acceptable carrier suitable for administration to an individual by injection. In some embodiments, the CR2 ligand is Epstein-Barr Virus (EBV), and the non-antibody polypeptides competitively inhibit EBV binding to CR2 protein in a standard assay. In some embodiments, the CR2 ligand is EBV gp350, and the non-antibody polypeptides competitively inhibit EBV binding to CR2 protein in a standard assay. In any of the embodiments described herein, the standard assay is a competitive ELISA. In some embodiments, the CR2 ligand is one or more C3 fragments and the non-antibody polypeptides competitively inhibit the binding of one or more C3 fragments to CR2 protein in a standard assay. In some embodiments, the CR2 ligand is selected from the group consisting of C3d, C3dg, and iC3b and the non-antibody polypeptides competitively inhibit the binding of one or more C3 fragments to CR2 protein in a standard assay. In some embodiments, the CR2 ligand is C3d and the non-antibody polypeptides competitively inhibit the binding of C3d to CR2 protein in a standard assay.

In some embodiments, the pharmaceutical compositions comprise any one or more non-antibody polypeptides that selectively bind to EBV and competitively inhibit EBV binding to CR2 protein in a standard assay and a pharmaceutically acceptable carrier suitable for administration to an individual. In some embodiments, the pharmaceutical compositions comprise any one or more non-antibody polypeptides that selectively bind to EBV and competitively inhibit EBV binding to CR2 protein in a standard assay and a pharmaceutically acceptable carrier suitable for oral administration to an individual. In some embodiments, the pharmaceutical compositions comprise any one or more non-antibody polypeptides that selectively bind to EBV and competitively inhibit EBV binding to CR2 protein in a standard assay and a pharmaceutically acceptable carrier suitable for administration to an individual by injection. In some embodiments, the non-antibody polypeptides selectively bind to EBV gp350 and competitively inhibit EBV binding to CR2 protein in a standard assay. In any of the embodiments described herein, the standard assay is a competitive ELISA.

In some embodiments, the pharmaceutical compositions comprise any one or more non-antibody polypeptides that selectively bind to CR2 protein and competitively inhibit EBV binding to CR2 protein in a standard assay and a pharmaceutically acceptable carrier suitable for administration to an individual. In some embodiments, the pharmaceutical compositions comprise any one or more non-antibody polypeptides that selectively bind to CR2 protein and competitively inhibit EBV binding to CR2 protein in a standard assay and a pharmaceutically acceptable carrier suitable for oral administration to an individual. In some embodiments, the pharmaceutical compositions comprise any one or more non-antibody polypeptides that selectively bind to CR2 protein and competitively inhibit EBV binding to CR2 protein in a standard assay and a pharmaceutically acceptable carrier suitable for administration to an individual by injection. In any of the embodiments described herein, the standard assay is a competitive ELISA.

In some embodiments, the pharmaceutical compositions comprise a non-antibody polypeptide of the amino acid sequence CSEGSLKGC (SEQ ID NO: 20) that selectively binds CR2 protein and competitively inhibits EBV binding to CR2 protein in a standard assay, and a pharmaceutically acceptable carrier suitable for administration to an individual. In some embodiments, the pharmaceutical compositions comprise a non-antibody polypeptide of the amino acid sequence CSEGSLKGC (SEQ ID NO: 20) that selectively binds CR2 protein and competitively inhibits EBV binding to CR2 protein in a standard assay, and a pharmaceutically acceptable carrier suitable for oral administration to an individual. In some embodiments, the pharmaceutical compositions comprise a non-antibody polypeptide of the amino acid sequence CSEGSLKGC (SEQ ID NO: 20) that selectively binds CR2 protein and competitively inhibits EBV binding to CR2 protein in a standard assay, and a pharmaceutically acceptable carrier suitable for administration to an individual by injection.

In some embodiments, the pharmaceutical compositions comprise any one or more non-antibody polypeptides that selectively bind to one or more C3 fragments and competitively inhibit the binding of one or more C3 fragments to CR2 protein in a standard assay, and a pharmaceutically acceptable carrier suitable for administration to an individual. In some embodiments, the pharmaceutical compositions comprise any one or more non-antibody polypeptides that selectively bind to one or more C3 fragments and competitively inhibit the binding of one or more C3 fragments to CR2 protein in a standard assay, and a pharmaceutically acceptable carrier suitable for oral administration to an individual. In some embodiments, the pharmaceutical compositions comprise any one or more non-antibody polypeptides that selectively bind to one or more C3 fragments and competitively inhibit the binding of one or more C3 fragments to CR2 protein in a standard assay and a pharmaceutically acceptable carrier suitable for administration to an individual by injection. In some embodiments, the C3 fragments include, but are not limited to, C3d, C3dg, and iC3b. In some embodiments, the non-antibody polypeptides selectively bind to C3d and competitively inhibit the binding of C3d to CR2 protein in a standard assay. In any of the above embodiments, the standard assay is a competitive ELISA.

In some embodiments, the pharmaceutical compositions comprise any one or more non-antibody polypeptides that selectively bind to CR2 protein and competitively inhibit the binding of one or more C3 fragments to CR2 protein in a standard assay, and a pharmaceutically acceptable carrier suitable for administration to an individual. In some embodiments, the pharmaceutical compositions comprise any one or more non-antibody polypeptides that selectively bind to CR2 protein and competitively inhibit the binding of one or more C3 fragments to CR2 protein in a standard assay, and a pharmaceutically acceptable carrier suitable for oral administration to an individual. In some embodiments, the pharmaceutical compositions comprise any one or more non-antibody polypeptides that selectively bind to CR2 protein and competitively inhibit the binding of one or more C3 fragments to CR2 protein in a standard assay and a pharmaceutically acceptable carrier suitable for administration to an individual by injection. In some embodiments, the C3 fragments include, but are not limited to, C3d, C3dg, and iC3b. In some embodiments, the non-antibody polypeptides selectively bind to CR2 protein and competitively inhibit the binding of C3d to CR2 protein in a standard assay. In any of the above embodiments, the standard assay is a competitive ELISA.

In some embodiments, the pharmaceutical compositions comprise non-antibody polypeptides that selectively bind CR2 protein, competitively inhibit binding of one or more C3 fragments to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for administration to an individual. In some embodiments, the one or more C3 fragments include, but are not limited to, C3d, C3dg, and iC3b.

In some embodiments, the pharmaceutical compositions comprise non-antibody polypeptides that selectively bind CR2 protein, competitively inhibit binding of one or more C3 fragments to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for oral administration to an individual. In some embodiments, the one or more C3 fragments include, but are not limited to, C3d, C3dg, and iC3b.

In some embodiments, the pharmaceutical compositions comprise non-antibody polypeptides that selectively bind CR2 protein, competitively inhibit binding of one or more C3 fragments to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for administration to an individual by injection. In some embodiments, the one or more C3 fragments include, but are not limited to, C3d, C3dg, and iC3b.

In some embodiments, the pharmaceutical compositions comprise non-antibody polypeptides that selectively bind CR2 protein, competitively inhibit binding of C3d to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for administration to an individual.

In some embodiments, the pharmaceutical compositions comprise non-antibody polypeptides that selectively bind CR2 protein, competitively inhibit binding of C3d to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for oral administration to an individual.

In some embodiments, the pharmaceutical compositions comprise non-antibody polypeptides that selectively bind CR2 protein, competitively inhibit binding of C3d to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for administration to an individual by injection.

In some embodiments, the pharmaceutical compositions comprise any one or more non-antibody polypeptides that selectively bind to a complement receptor type 2 (CR2) protein, a ligand thereof, or both, wherein the CR2 ligand is IFN-α, wherein the non-antibody polypeptides competitively inhibit IFN-α-binding to CR2 protein in a standard assay and a pharmaceutically acceptable carrier suitable for administration to an individual. In some embodiments, the pharmaceutical compositions comprise any one or more non-antibody polypeptides that selectively bind to a complement receptor type 2 (CR2) protein, a ligand thereof, or both, wherein the CR2 ligand IFN-α, wherein the non-antibody polypeptides competitively inhibit IFN-α-binding to CR2 protein in a standard assay and a pharmaceutically acceptable carrier suitable for oral administration to an individual. In some embodiments, the pharmaceutical compositions comprise any one or more non-antibody polypeptides that selectively bind to a complement receptor type 2 (CR2) protein, a ligand thereof, or both, wherein the CR2 ligand IFN-α, wherein the non-antibody polypeptides competitively inhibit IFN-α-binding to CR2 protein in a standard assay and a pharmaceutically acceptable carrier suitable for administration to an individual by injection. In some embodiments, the non-antibody polypeptides bind to IFN-α and competitively inhibit IFN-α-binding to CR2 protein in a standard assay. In some embodiments, the non-antibody polypeptides bind to CR2 protein and competitively inhibit IFN-α-binding to CR2 protein in a standard assay. In some embodiments, the non-antibody polypeptides bind to a site in SCRs 1 and 2 of CR2 protein and competitively inhibit IFN-α-binding to CR2 protein in a standard assay. In any of the embodiments described herein, the standard assay is a competitive ELISA.

In some embodiments, the pharmaceutical compositions comprise non-antibody polypeptides that selectively bind to a complement receptor type 2 (CR2) protein, a ligand thereof, or both, wherein the CR2 ligand is FcεRII/CD23, wherein the non-antibody polypeptides competitively inhibit FcεRII/CD23 binding to CR2 and a pharmaceutically acceptable carrier suitable for administration to an individual. In some embodiments, the pharmaceutical compositions comprise non-antibody polypeptides that selectively bind to a complement receptor type 2 (CR2) protein, a ligand thereof, or both, wherein the CR2 ligand is FcεRII/CD23, wherein the non-antibody polypeptides competitively inhibit FcεRII/CD23 binding to CR2 and a pharmaceutically acceptable carrier suitable for oral administration to an individual. In some embodiments, the pharmaceutical compositions comprise non-antibody polypeptides that selectively bind to a complement receptor type 2 (CR2) protein, a ligand thereof, or both, wherein the CR2 ligand is FcεRII/CD23, wherein the non-antibody polypeptides competitively inhibit FcεRII/CD23 binding to CR2 and a pharmaceutically acceptable carrier suitable for administration to an individual by injection. In some embodiments, the non-antibody polypeptides bind to FcεRII/CD23 and competitively inhibit FcεRII/CD23-binding to CR2 protein in a standard assay. In some embodiments, the non-antibody polypeptides bind to CR2 protein and competitively inhibit FcεRII/CD23-binding to CR2 protein in a standard assay. In some embodiments, the non-antibody polypeptides bind to a site in SCRs 1 and 2 of CR2 protein and competitively inhibit FcεRII/CD23-binding to CR2 protein in a standard assay. In any of the embodiments described herein, the standard assay is a competitive ELISA.

The compositions of the present invention include bulk drug compositions useful in the manufacture of non-pharmaceutical compositions (e.g., impure or non-sterile compositions) and pharmaceutical compositions (i.e. compositions that are suitable for administration in a subject, individual, or a patient) and can be packaged in unit dosage forms, such as single unit dosages or multidosage forms. Pharmaceutical compositions within the scope of the present invention can also contain other compounds that may be biologically active or inactive, such as pharmaceutically acceptable preservatives, buffers, tonicity agents, antioxidants and/or stabilizers.

The pharmaceutical compositions may be formulated as sterile, substantially isotonic solutions in full compliance with all Good Manufacturing Practice (GMP) regulations of the U.S. Food and Drug Administration. In certain embodiments, the composition is free of pathogen.

The compositions of the invention may be administered using any medically appropriate procedure, such as, for example, injection (e.g., intravenous, intra-arterial, parenteral, intraocular, and the like).

The pharmaceutical compositions described herein can be formulated for any appropriate manner of administration, including, but not limited to, oral, inhaled, buccal, rectal, parenteral, nasal, intranasal, topical, intravenous, intra-arterial, subdermal, intracranial, ophthalmic (e.g., topical administration to the eye, intraocular injection (e.g., subconjunctival, subtenon, intravitreal, and the like), or implantation), or intrathecal administration. In some embodiments, parenteral routes include, but are not limited to, subcutaneous, intradermal, transdermal, intravenous, intramuscular, and intraperitoneal routes. Intravenous, intraperitoneal, intradermal, subcutaneous, and intramuscular administration can be performed using standard methods known in the art. Inhalation (aerosol) delivery can also be performed using standard methods known in the art. The pharmaceutical compositions may be administered systemically or locally, for example, via regional administration, intramural administration, or use of an implant that retains the composition at the site of implantation.

Formulation for oral administration can take the form of, for example, tablets or capsules prepared by conventional means with pharmaceutically acceptable excipients such as binding agents (e.g., pregelatinized starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). Biodegradable microspheres (e.g., polylactate polyglycolate) may also be employed as carriers for the pharmaceutical compositions of the present invention. The preparations can also contain buffer salts, flavoring, coloring and sweetening agents as appropriate.

The compositions can be formulated for injection. In certain embodiments, the pharmaceutical compositions provided herein are formulated for intravenous, parenteral intraperitoneal, or intraocular injection. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Formulation for parenteral administration can take the form of, for example, aqueous and non-aqueous, isotonic sterile injection solutions, which can contain antioxidants, buffers, bacteriostatic compounds, and solutes that render the formulation isotonic with the blood of the intended recipient. Parenteral formulations can also comprise aqueous and non-aqueous sterile suspensions including suspending agents, solubilizers, thickening agents, stabilizers, and preservatives.

The compositions described herein may be administered as part of a sustained release formulation (i.e., a formulation that effects a slow release of compound following administration). Such formulations can be administered by a variety of routes, including, but not limited to, oral, rectal, or subcutaneous implantation, or by implantation at the desired target site. Sustained-release formulations may contain a non-antibody polypeptide dispersed in a carrier matrix and/or contained within a reservoir surrounded by a release rate-controlling membrane. Carriers for use with such formulations may be biocompatible and/or biodegradable and capable of providing a relatively constant level of active component release.

In some embodiments, the non-antibody polypeptides of the present invention may be entrapped in microcapsules prepared, for example, by coacervation techniques or by interfacial polymerization (e.g., hydroxymethylcellulose or gelatin-microcapsules and poly(methylmethacylate) microcapsules), in colloidal drug delivery systems (e.g., liposomes, albumin microspheres, microemulsions, nano-particles and nanocapsules), or in macroemulsions, as understood by one skilled in the art. The non-antibody polypeptides disclosed herein may also be formulated as liposomes.

In some embodiments, administration of the non-antibody polypeptide of the present invention can be achieved in vivo, in vitro, and ex vivo in various ways. As used herein, ex vivo refers to performing part of the administration process outside of a patient or a subject, such as by transfecting a population of cells removed from a patient or subject with a recombinant molecule comprising a nucleic acid sequence encoding a non-antibody polypeptide according to the present invention under conditions such that the recombinant molecule is subsequently expressed by the transfected cell, and returning the transfected cells to the patients. In vitro and ex vivo routes of administering a composition to a culture of host cells can be accomplished by a method, including, but not limited to, transfection, transformation, electroporation, microinjection, lipofection, adsorption, protoplast fusion, use of protein carrying agents, use of ion carrying agents, use of detergents for cell permeabilization, and mixing (e.g., combining) a compound in culture with a target cell.

The pharmaceutical compositions provided herein optionally contain pharmaceutically acceptable preservatives, buffers, tonicity agents, antioxidants and/or stabilizers known to those skilled in the art. Suitable preservatives for use in a solution include polyquaternium-1, benzalkonium chloride, thimerosal, chlorobutanol, methyl paraben, propyl paraben, phenylethyl alcohol, disodium-EDTA, sorbic acid, benzethonium chloride, and the like. Typically (but not necessarily) such preservatives are employed at a level of from 0.001% to 1.0% by weight. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents include dextran 40, dextran 70, dextrose, glycerin, potassium chloride, propylene glycol, sodium chloride, and the like, such that the sodium chloride equivalent of the injectable solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like

In some embodiments, the non-antibody polypeptide of the present invention can be complexed with various well-known compounds that enhance the in vivo stability of the non-antibody polypeptide, or otherwise enhance its pharmacological properties (e.g., increase the half-life of the non-antibody polypeptide, reduce its toxicity, and enhance solubility or uptake). Examples of such modifications or complexing agents include sulfate, gluconate, citrate, and phosphate. The non-antibody polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations suitable for various routes of administration can be found in REMINGTON'S PHARMACEUTICAL SCIENCES, Mace Publishing Company, Philadelphia, Pa., 17th ed. (1985). For a brief review of methods for drug delivery, see also Langer, Science 249:1527-1533 (1990).

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process.

In some embodiments, any of the pharmaceutical compositions provided herein are formulated as single or unit doses for administration by any of the routes or methods described herein. In some embodiments, a single dose of a non-antibody compound (e.g., a non-antibody polypeptide) for administration by any of the routes or methods described herein comprises between about 0.01 μg/kg and about 10 mg/kg of body weight of an individual. In some embodiments, a single dose of a non-antibody compound (e.g., a non-antibody polypeptide) for administration by any of the routes or methods described herein comprises between about 1 μg/kg and about 10 mg/kg of body weight of an individual. In some embodiments, a single dose of a non-antibody compound (e.g., a non-antibody polypeptide) for administration by any of the routes or methods described herein comprises between about 5 μg/kg and about 7 mg/kg of body weight of an individual. In some embodiments, a single dose of a non-antibody compound (e.g., a non-antibody polypeptide) for administration by any of the routes or methods described herein comprises between about 10 μg/kg and about 5 mg/kg of body weight of an individual. In some embodiments, a single dose of a non-antibody compound (e.g., a non-antibody polypeptide) for administration by any of the routes or methods described herein comprises between about 0.1 mg/kg and about 5 mg/kg of body weight of an individual. In some embodiments, a single dose of a non-antibody compound (e.g., a non-antibody polypeptide) for administration by any of the routes or methods described herein comprises between about 0.1 μg/kg and about 10 μg/kg of body weight of an individual.

In some embodiments, a single dose of a non-antibody compound (e.g., a non-antibody polypeptide) for administration by any of the routes or methods described herein comprises between about 1 ng/kg and about less than 1 mg/kg of body weight of an individual. In some embodiments, a single dose of a non-antibody compound (e.g., a non-antibody polypeptide) for administration by any of the routes or methods described herein comprises between about 20 ng/kg and about 600 μg/kg of body weight of the individual. In some embodiments, a single dose of a non-antibody compound (e.g., a non-antibody polypeptide) for administration by any of the routes or methods described herein comprises between about 20 ng/kg and about 600 μg/kg of body weight of the individual, between about 20 ng/kg and about 500 μg/kg, between about 20 ng/kg and about 400 μg/kg, between about 20 ng/kg and about 300 μg/kg, between about 20 ng/kg and about 200 μg/kg, between about 20 ng/kg and about 100 μg/kg, and between about 20 ng/kg and about 50 μg/kg of body weight of the individual.

In some embodiments, the non-antibody compound (e.g., a non-antibody polypeptide) is administered by any route of administration described herein at a dose of less than about 500 μg of non-antibody polypeptide per milliliter of formulation, less than about 250 μg of non-antibody polypeptide per milliliter of formulation, less than about 100 μg of non-antibody polypeptide per milliliter of formulation, less than about 50 μg of non-antibody polypeptide per milliliter of formulation, less than about 40 μg of non-antibody polypeptide per milliliter of formulation, less than about 30 μg of non-antibody polypeptide per milliliter of formulation, less than about 20 μg of non-antibody polypeptide per milliliter of formulation, less than about 10 μg of non-antibody polypeptide per milliliter of formulation, and between about 5 μg of non-antibody polypeptide and about 10 μg of non-antibody polypeptide per milliliter of formulation.

With particular regard to the methods described herein, an effective amount of a non-antibody compound (e.g., a non-antibody polypeptide), to administer to an individual is an amount that measurably inhibits (or prevents) histological damage, including oxidative damage or cell death, in the individual as compared to in the absence of administration of the agent. A suitable single dose of a non-antibody compound (e.g., a non-antibody polypeptide) to administer to an individual is a dose that is capable of reducing or preventing at least one symptom, type of injury, or resulting damage, from any one or more of the disease indications disclosed herein in an individual when administered one or more times over a suitable time period. Suitable doses of non-antibody compounds (e.g., non-antibody polypeptides), including for various routes of administration, are described in detail above. In one aspect, an effective amount of an agent that inhibits CR2 activation to administer to an individual comprises an amount that is capable of inhibiting at least one symptom of a disease characterized by CR2 activation (e.g., an autoimmune disease) without being toxic to the individual.

One of skill in the art will be able to determine that the number of doses of an agent to be administered to an individual is dependent upon the extent of the disease being treated and/or the anticipated or observed physiological damage associated with it, as well as the response of an individual patient to the treatment. The clinician will be able to determine the appropriate timing for delivery of the non-antibody compounds in a manner effective to reduce the symptom(s) associated with disease being treated in the individual.

### Kits and Articles of Manufacture

In another aspect, kits and articles of manufacture comprising the non-antibody polypeptides described herein are also provided.

In some embodiments, the kits and articles of manufacture provided herein comprise any of the pharmaceutical formulations comprising non-antibody polypeptides described herein. For example, the kits and articles of manufacture can comprise a non-antibody polypeptide of the amino acid sequence CSEGSLKGC (SEQ ID NO: 20) that selectively binds CR2 protein and competitively inhibits EBV binding to CR2 protein in a standard assay, and a pharmaceutically acceptable carrier suitable for administration to an individual by any route of administration, including oral administration or injection.

The kits and articles of manufacture can also comprise one or more non-antibody polypeptides that selectively bind CR2 protein, competitively inhibit binding of C3d to CR2 protein in a standard assay and comprise a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13), and a pharmaceutically acceptable carrier suitable for administration to an individual by any route of administration, including oral administration or injection.

In some embodiments, the article of manufacture comprises a labeled container. Suitable containers include, but are not limited to, bottles, vials, ampoules, and test tubes. The containers may be formed from various materials capable of being sterilized, such as plastic or glass. In some embodiments, the container contains a composition useful for the treatment of a condition or a disease associated with amplified B cell responses or an infection mediated by a CR2 ligand. In some embodiments, the label on the container indicates that the composition useful for the treatment of a disease associated with amplified B cell responses or an infection mediated by a CR2 ligand. In some embodiments, the kit comprises pharmaceutical compositions as described herein in lyophilized or powder form. In some embodiments, the kits further comprise a second container comprising sterile water or buffer suitable for reconstituting a lyophilized or powdered pharmaceutical composition for administration to an individual. In some embodiments, the kit further comprises a package insert with instructions for use of the kit in conjunction with the methods described herein.

### Methods of Using the Non-Antibody Polypeptides

In another aspect, provided herein are methods useful in a variety of applications including, but not limited to, treatment of autoimmune diseases and vaccine preparation. Methods of inhibiting or reducing a biological response mediated by a CR2 ligand or of inhibiting or reducing the binding of a CR2 protein to its ligand are also provided.

Provided herein are methods of inhibiting or reducing a biological response mediated by a CR2 ligand in an individual. In some embodiments, the methods of inhibiting or reducing a biological response mediated by a CR2 ligand in an individual comprise administering an effective amount of a composition comprising one or more non-antibody polypeptides of the described herein to the individual.

As used herein, a “biological response mediated by a CR2 ligand” includes, but is not limited to, an autoimmune response (or autoimmunity), a humoral immune response, EBV infection, immunoglobulin (Ig) class switching (e.g., switching from IgG to IgE during hypersensitivity reactions of diseases), B cell activation, T cell activation, binding of antigens to follicular dendritic cells, binding of antigens or EBV to epithelial cells, IFN-a induced mRNA changes, B cell adherence to sites of inflammation, and CR2-dependent human immunodeficiency virus-1 (HIV-1) infection.

“Autoimmune response” or “autoimmunity,” as used herein, refers to a subset of humoral immune responses that are mediated by CR2 interactions with its ligand(s). Autoimmune response is an immune response directed against an individual's own body (e.g., against tissues, cells, proteins, nucleic acids, lipids, or any other biomolecule) as a result of the failure of an individual's ability to recognize its own cells and tissues (i.e., a failure of immunological tolerance). An autoimmune response can be demonstrated by the presence of autoantibodies or T lymphocytes reactive with an individual's own antigens. Several mechanisms have been proposed for the pathogenesis of an autoimmune response (autoimmunity) and autoimmune disease. For example, B cells are thought to play key roles in an autoimmune response, including synthesizing pathogenic autoantibodies, antigen presentation to pathogenic T cells, and regulating the development and function of T cells. Downregulation of B cells and autoimmune responses to autoantigens by interruption of essential CR2/CD19 co-activation functions is one mechanism for treatment of autoimmune disease (i.e. non-depleting B cell therapeutics). Kaya et al. (2001) Nature Immunol. 2:739-745; Kuhn et al. (2006) J. Clin. Invest. 116; 961-973.

Autoimmune diseases include, but are not limited to, ankylosing spondylitis, autoimmune hemolytic anemia, autoimmune inner ear disease, bullous pemphigoid, celiac disease, chagas disease, chronic obstructive pulmonary disease (COPD), dermatomylositis, endometriosis, hidradenitis suppurativa, interstitial cystitis, morphea, narcolepsy, neuromyotonia, pemphigus vulgaris, pernicious anaemia, polymyositis, primary biliary cirrhosis, schizophrenia, scleroderma, vasculitis, temporal arterities, vitiligo, Wegener's granulomatosis, myasthenia gravis, Alzheimer's disease, multiple sclerosis, rheumatoid arthritis, systemic lupus erythematosus, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barré syndrome, Hashimoto's disease, idiopathic thrombocytopenic purpura, thrombotic thrombocytopenic purpura, atypical hemolytic uremic syndrome, Sjögren's syndrome, Takayasu's arteritis, and chronic fatigue syndrome.

In one aspect, the present invention provides methods of inhibiting or reducing an autoimmune response mediated by a CR2 ligand in an individual comprising administering an effective amount of a composition comprising one or more of the non-antibody compounds (e.g., non-antibody polypeptides) described herein to the individual. In some embodiments, the methods are methods of inhibiting or reducing an autoimmune response mediated by a C3 fragment (e.g., C3d, iC3b, or C3dg) in an individual comprising administering an effective amount of a composition comprising a non-antibody compound (e.g., a non-antibody polypeptide) that selectively binds to a CR2 protein, a ligand thereof, or both, wherein the ligand is a C3 fragment, and wherein the non-antibody compound (e.g., a non-antibody polypeptide) competitively inhibits binding of the C3 fragment to an SCR domain of CR2 protein in a standard assay. In some embodiments, the non-antibody compound (i.e., a non-antibody polypeptide) selectively binds to a C3 fragment and competitively inhibits binding of the C3 fragment to an SCR domain of CR2 protein in a standard assay. In some embodiments, the non-antibody compound (e.g., a non-antibody polypeptide) selectively binds to CR2 protein and competitively inhibits binding of the C3 fragment to an SCR domain of CR2 protein in a standard assay. In some embodiments, the SCR domain of CR2 protein comprises SCR 1. In some embodiments, the SCR domain of CR2 protein comprises SCR2. In some embodiments, the SCR domain of CR2 protein comprises SCRs 1 and 2. In some embodiments, the SCR domain of CR2 protein comprises SCRs 1 and 2 and the eight amino acid residue linker region connecting SCR1 and SCR2. In some embodiments, the C3 fragment is selected from the group consisting of iC3b, C3dg and C3d. In some embodiments, the C3 fragment is C3d. In some embodiments, the non-antibody compounds (e.g., non-antibody polypeptides) that selectively bind CR2 protein and competitively inhibit binding of a C3 fragment to CR2 protein in a standard assay comprise non-antibody polypeptides having a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13). In any of the above embodiments, the standard assay is a competitive ELISA.

In another aspect, provided herein are methods of inhibiting or reducing a humoral immune response mediated by a CR2 ligand in an individual comprising administering an effective amount of a composition comprising one or more of the non-antibody compounds (e.g., a non-antibody polypeptide) described herein to the individual. In some embodiments, the present invention provides a method of inhibiting or reducing a humoral immune response mediated by a C3 fragment (e.g., iC3b, C3dg and C3d) in an individual comprising administering to the individual an effective amount of a composition comprising a non-antibody compound (e.g., a non-antibody polypeptide) that selectively binds to a CR2 protein, a ligand thereof, or both, wherein the ligand is a C3 fragment, and wherein the non-antibody compound (e.g., a non-antibody polypeptide) competitively inhibits binding of the C3 fragment to an SCR domain in a standard assay (e.g., a competitive ELISA).

A “humoral immune response mediated by a CR2 ligand,” as used herein, refers to an immune response consisting of secreted antibodies produced by B cells amplified by the binding of a cell-bound C3 fragment to CR2 protein, followed by costimulation of the B cell receptor via the bound cell (antigen). Cross-linking of the CR2 protein to the B cell receptor by C3 fragments such as iC3b, C3dg and C3d amplifies a signal transduction cascade through the CR2/CD19/CD81 co-activation complex. CR2-mediated humoral immune responses are associated with a variety of conditions, including infectious disease. Such diseases include, for example, HIV-1 and other chronic bacterial, viral, fungal, mycoplasmal and other pathogens for which humoral immunity is key to protection. These diseases also include, but are not limited to, hypersensitivity reactions, asthma and other inflammatory diseases that are associated or caused by environmental allergen/antigen exposures which drive B cell responses in a CR2-dependent manner. Thus, provided herein are methods of inhibiting or reducing a humoral response mediated by a CR2 ligand in an individual comprising administering an effective amount of a composition comprising one or more of the non-antibody compounds (e.g., a non-antibody polypeptide) described herein to the individual. In some embodiments, the methods are methods of inhibiting or reducing a humoral response mediated by a C3 fragment (e.g., C3d, iC3b, or C3dg) in an individual comprising administering an effective amount of a composition comprising a non-antibody compounds (e.g., non-antibody polypeptides) that selectively bind to a CR2 protein, a ligand thereof, or both, wherein the ligand is a C3 fragment, and wherein the non-antibody compounds (e.g., non-antibody polypeptides) competitively inhibit binding of the C3 fragment to an SCR domain of CR2 protein in a standard assay. In some embodiments, the non-antibody compounds (e.g., non-antibody polypeptides) selectively bind to a C3 fragment and competitively inhibit binding of the C3 fragment to an SCR domain of CR2 protein in a standard assay. In some embodiments, the non-antibody compounds (e.g., non-antibody polypeptide) selectively bind to CR2 protein and competitively inhibit binding of the C3 fragment to an SCR domain of CR2 protein in a standard assay. In some embodiments, the SCR domain of CR2 protein comprises SCR 1. In some embodiments, the SCR domain of CR2 protein comprises SCR2. In some embodiments, the SCR domain of CR2 protein comprises SCRs 1 and 2. In some embodiments, the SCR domain of CR2 protein comprises SCRs 1 and 2 and the eight amino acid residue linker region connecting SCR1 and SCR2. In some embodiments, the C3 fragment is selected from the group consisting of iC3b, C3dg and C3d. In some embodiments, the C3 fragment is C3d. In some embodiments, the non-antibody compounds (e.g., non-antibody polypeptides) that selectively bind CR2 protein and competitively inhibit binding of a C3 fragment to CR2 protein in a standard assay comprise non-antibody polypeptides having a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13). In any of the above embodiments, the standard assay is a competitive ELISA.

In another aspect, provided herein are methods of inhibiting or reducing CR2-dependent human immunodeficiency virus-1 (HIV-1) infection mediated by a CR2 ligand in an individual comprising administering an effective amount of a composition comprising a non-antibody compound (e.g., a non-antibody polypeptide) of the present invention to the individual.

HIV infection correlates in vivo with high levels of complement activation, as shown by both a decrease of serum complement components and an increase of complement activation fragments such as iC3b, C3b, and C3d in the blood. HIV-negative normal human serum has been shown in vitro to promote HIV infection of cells bearing complement receptors such as CR2 at low viral titers. G. Senaldi et al., J. Infect. Dis. (1990) 162:1227. CR2 protein has been shown to mediate interaction of C3-bound HIV-1 as an immune complex with B cells in a fashion that promotes transfer of virus and infection of CD4⁺ T cells. S. Moir et al., J. Exp. Med. (2000) 192:637-646.

Thus, in some embodiments, the present invention provides methods of inhibiting CR2-dependent HIV-1 infection mediated by a C3 fragment (e.g., C3d, iC3b, or iC3b) of B cells, follicular dendritic cells (FDC) in lymph nodes, and CD4⁺ T cells in an individual comprising administering to the individual an effective amount of a composition comprising a non-antibody compound (e.g., a non-antibody polypeptide) that selectively binds to a CR2 protein, a ligand thereof, or both, wherein the ligand is a C3 fragment, and wherein the non-antibody compound (e.g., a non-antibody polypeptide) competitively inhibits binding of one or more C3 fragments to an SCR domain of CR2 protein in a standard assay (e.g., a competitive ELISA). In some embodiments, the non-antibody compound (e.g., a non-antibody polypeptide) selectively binds to a C3 fragment and competitively inhibits binding of one or more C3 fragments to an SCR domain of CR2 protein in a standard assay. In some embodiments, the non-antibody compound (e.g., a non-antibody polypeptide) selectively binds to CR2 protein and competitively inhibits binding of one or more C3 fragments to an SCR domain of CR2 protein in a standard assay. In some embodiments, the SCR domain of CR2 protein comprises SCR 1. In some embodiments, the SCR domain of CR2 protein comprises SCR2. In some embodiments, the SCR domain of CR2 protein comprises SCRs 1 and 2. In some embodiments, the SCR domain of CR2 protein comprises SCRs 1 and 2 and the eight amino acid residue linker region connecting SCR1 and SCR2. In some embodiments, the C3 fragment is selected from the group consisting of iC3b, C3dg and C3d. In some embodiments, the C3 fragment is C3d. In some embodiments, the non-antibody compounds (e.g., non-antibody polypeptides) that selectively bind CR2 protein and competitively inhibit binding of a C3 fragment to CR2 protein in a standard assay comprise non-antibody polypeptides having a sequence selected from the group consisting of: a) amino acid sequence APQXLSSQYSRT (SEQ ID NO: 7), wherein X is H or A; b) amino acid sequence APXHLSSQ (SEQ ID NO: 8), wherein X is Q or A; c) amino acid sequence ISTSNPRX₁X₂STA (SEQ ID NO: 9), wherein X₁ is H or A, and X₂ is H or A; d) amino acid sequence ISTSNPRH (SEQ ID NO: 10); e) amino acid sequence IATANPRHHSTA (SEQ ID NO: 10); e) amino acid sequence IATANPRHHSTA (SEQ ID NO: 11); f) amino acid sequence CDPKNHVHC (SEQ ID NO: 12); and g) amino acid sequence DPKNHVH (SEQ ID NO: 13). In any of the above embodiments, the standard assay is a competitive ELISA.

In another aspect, provided herein are methods of inhibiting or reducing an EBV infection mediated by a CR2 ligand in an individual comprising administering an effective amount of a composition comprising a non-antibody polypeptide of the present invention described herein to the individual. In some embodiments, the present invention provides methods of inhibiting EBV infection of B cells and epithelial cells mediated by EBV gp350 in an individual, comprising administering to the individual an effective amount of a composition comprising a non-antibody polypeptide that selectively binds to a CR2 protein, a ligand thereof, or both, wherein the ligand is EBV gp350, and wherein the non-antibody polypeptide competitively inhibits binding of EBV to an SCR domain of CR2 protein in a standard assay (e.g., a competitive ELISA).

An “EBV infection,” as used herein, refers to an infection by Epstein Barr virus, a human gamma-herpesvirus, that is associated with a number of diseases including, but not limited to, B-cell lymphomas, African Burkitt's lymphoma, nasopharyngeal carcinoma, EBV-associated lymphoproliferative disease, non-Hodgkin's lymphoma, oral hairy leukoplakia, a human immunodeficiency virus-1 (HIV-1) related lymphoproliferative disease, and an autoimmune disease. Autoimmune disease mediated by the EBV infection includes, but is not limited to, systemic lupus erythematosus, rheumatoid arthritis, multiple sclerosis, and chronic fatigue syndrome.

In some embodiments, provided herein are methods of inhibiting or reducing an EBV infection mediated by a CR2 ligand in an individual comprising administering an effective amount of a composition comprising one or more of the non-antibody compounds (e.g., non-antibody polypeptides) described herein to the individual. In some embodiments, the CR2 ligand is EBV gp350. In some embodiments, provided herein are methods of inhibiting EBV infection of B cells and epithelial cells mediated by EBV gp350 in an individual, comprising administering to the individual an effective amount of a composition comprising one or more non-antibody compounds (e.g., non-antibody polypeptides) that selectively bind to a CR2 protein, a ligand thereof, or both, wherein the ligand is EBV gp350, and wherein the non-antibody compound (e.g., a non-antibody polypeptide) competitively inhibits binding of EBV to an SCR domain of CR2 protein in a standard assay (e.g., a competitive ELISA). In some embodiments, the non-antibody compound (e.g., a non-antibody polypeptide) selectively binds to EBV gp350 and competitively inhibits binding of EBV to an SCR domain of CR2 protein in a standard assay. In some embodiments, the non-antibody compound (e.g., a non-antibody polypeptide) selectively binds to CR2 protein and competitively inhibits binding of EBV to an SCR domain of CR2 protein in a standard assay. In some embodiments, the SCR domain of CR2 protein comprises SCR 1. In some embodiments, the SCR domain of CR2 protein comprises SCR2. In some embodiments, the SCR domain of CR2 protein comprises SCRs 1 and 2. In some embodiments, the SCR domain of CR2 protein comprises SCRs 1 and 2 and the eight amino acid residue linker region connecting SCR1 and SCR2. In some embodiments, the non-antibody compound (e.g., a non-antibody polypeptide) that selectively binds EBV gp350 and competitively inhibits EBV binding to CR2 protein in a standard assay. In some embodiments, the non-antibody compound (e.g., a non-antibody polypeptide) competitively inhibits EBV binding to CR2 protein in a standard assay and selectively binds to a binding site on EBV gp350 comprising two N-terminal β-barrel domains (D1 and D2) (see, e.g., FIG. 1) and a linker sequence between D1 and D2 (linker-1). In some embodiments, the non-antibody compound (e.g., a non-antibody polypeptide) comprises the amino acid sequence CSEGSLKGC (SEQ ID NO: 20), selectively binds CR2 protein and competitively inhibits EBV binding to CR2 protein in a standard assay. In any of the above embodiments, the standard assay is a competitive ELISA.

In another aspect, provided herein are methods of inhibiting or reducing immunoglobulin class switching mediated by a CR2 ligand in an individual comprising administering an effective amount of a composition comprising a non-antibody compound (e.g., a non-antibody polypeptide) as described herein to the individual. In some embodiments, the present invention provides a method of inhibiting or reducing Ig class switching from IgG to IgE mediated by FcεRII/CD23 and/or IFN-a in an individual with asthma or other hypersensitivity disorder comprising administering to the individual an effective amount of a composition comprising a non-antibody compound (e.g., a non-antibody polypeptide) that selectively binds to a CR2 protein, a ligand thereof, or both, wherein the ligand is FcεRII/CD23 and/or IFN-a, and wherein the non-antibody polypeptide competitively inhibits FcεRII/CD23 and/or IFN-a binding to an SCR domain of CR2 protein in a standard assay (e.g., a competitive ELISA).

As used herein, the term “immunoglobulin class switching,” “isotype switching,” “isotypic commutation,” “class switch recombination,” refers to a mechanism that changes an antibody from one class to another. During this process, only the constant region of the heavy chain of the antibody is changed. The variable region of the heavy chain remains the same, so the antigen specificity of the immunoglobulin molecule therefore is not affected in this process. Class switching from IgG to IgE in a CR2-dependent manner is associated with the change from a protective to a pro-inflammatory response.

In some embodiments, the methods described herein further comprise the step of treating the individual in conjunction with an additional form of therapy. In some embodiments, the additional form of therapy is an anti-inflammatory therapy. In some embodiments, the additional form of therapy is an anti-cancer therapy. In some embodiments, the anti-cancer therapy is selected from the group consisting of chemotherapy, radiation, surgery, hormone therapy, and immunotherapy.

The invention further provides a method of inhibiting or reducing a CR2 protein from binding to its ligand in an individual, comprising administering to the individual an effective amount of a non-antibody polypeptide, wherein the non-antibody polypeptide selectively binds to a CR2 protein, a ligand thereof, or both, and wherein the non-antibody polypeptide competitively inhibits the CR2 ligand binding to a CR2 SCR domain in a standard assay. The methods of the invention also find use in combination with cells or tissues.

### Methods of Screening for Ligand-Specific Non-Antibody Compounds

In another aspect, methods of screening for ligand-specific non-antibody compounds (e.g., a non-antibody polypeptide or other compound) that inhibit CR2 protein activation are provided herein.

In some embodiments, provided herein are methods of screening for ligand-specific non-antibody compounds (e.g., non-antibody polypeptides) that selectively bind to CR2 protein, a ligand thereof, or both, wherein the non-antibody compounds (e.g., non-antibody polypeptides) competitively inhibit ligand binding to CR2 protein in a standard assay (e.g. a competitive ELISA). In some embodiments, the methods comprise the steps of: (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody compounds from a library of such compounds in the presence of one or more CR2 ligands; and (c) detecting binding of one or more candidate non-antibody compounds to the immobilized CR2 protein or biologically-active fragment thereof, (d) isolating the one or more candidate non-antibody compounds bound to the immobilized CR2 protein or biologically-active fragment thereof, and (e) testing the one or more isolated candidate non-antibody compounds for the ability to competitively inhibit binding of one or more CR2 ligands to CR2 protein or a biologically-active fragment thereof in a standard assay.

As described herein, a CR2 ligand binds to and activates the CR2 protein. CR2 ligands include, but are not limited to, a C3 fragment (e.g., C3d, C3dg, and iC3b), EBV gp350, FcεRII/CD23, or IFNα. In some embodiments, the C3 fragment is iC3b, C3dg, C3b, or C3d. Candidate non-antibody compounds that inhibit CR2 activation can be detected by their ability to bind CR2 protein and to compete with CR2 ligands for binding to an SCR domain of CR2 protein. Competition assays in the presence of a ligand-specific non-antibody compound that inhibits CR2 activation are described, for example, in Example 7.

As used herein, a library of candidate non-antibody compounds comprises various non-antibody compounds as described herein (e.g. a non-antibody polypeptides, oligopeptides, peptides, nucleic acids, carbohydrates or other small molecules, as defined herein) with various binding affinities for CR2 protein. In some embodiments, a library of inhibitors is a phage display library comprising non-antibody polypeptides. In some embodiments, the method of screening for a ligand-specific non-antibody compounds comprises random fragment library screening, structure-based in silico database mining, and phage display library screening.

In some embodiments, the invention provides methods of screening for ligand-specific non-antibody compounds that selectively bind to a CR2 protein, a ligand thereof, or both, wherein the ligand is Epstein-Barr virus (EBV) and wherein the non-antibody compounds competitively inhibit EBV-binding to a CR2 protein in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody compounds from a library of such compounds in the presence of one or more CR2 ligands; and (c) detecting binding of one or more candidate non-antibody compounds to the immobilized CR2 protein or biologically-active fragment thereof, (d) isolating the one or more candidate non-antibody compounds bound to the immobilized CR2 protein or biologically-active fragment thereof, and (e) testing the one or more isolated candidate non-antibody compounds for the ability to competitively inhibit binding of one or more CR2 ligands to CR2 protein or biologically-active fragment thereof in a standard assay. In some embodiments, the CR2 ligand is EBV gp350, and the methods are used to identify non-antibody compounds that competitively inhibit EBV binding to CR2 protein in a standard assay. In some embodiments, the non-antibody compounds competitively inhibit EBV gp350 binding to a CR2 SCR domain in a standard assay. In some embodiments, the non-antibody compounds selectively bind to a binding site on EBV gp350 comprising two N-terminal β-barrel domains (D1 and D2) and a linker sequence between D1 and D2 and competitively inhibit EBV binding to a CR2 protein in a standard assay. In some embodiments, the non-antibody compounds selectively bind to a binding site on EBV gp350 comprising amino acid positions E21, D22, E155, E201, D208, E210, and D296 in a wildtype EBV gp350 sequence or their equivalent positions in a variant EBV gp350 sequence, and competitively inhibit EBV binding to CR2 protein in a standard assay. In some embodiments, the non-antibody compounds selectively bind to a binding site on EBV gp350 comprising amino acid positions Y151, I160, and W162 in a wildtype EBV gp350 sequence or their equivalent positions in a variant EBV gp350 sequence and competitively inhibit EBV binding to CR2 protein in a standard assay. In some embodiments, the non-antibody compounds selectively bind to a binding site on EBV gp350 comprising amino acid positions E21, D22, E155, E201, D208, E210, D296, Y151, I160, and W162 in a wildtype EBV gp350 sequence or their equivalent positions in a variant EBV gp350 sequence and competitively inhibit EBV binding to CR2 protein in a standard assay.

In some embodiments, the invention provides methods of screening for ligand-specific non-antibody compounds that selectively bind to a CR2 protein and competitively inhibit EBV (e.g. EBV gp350) binding to a CR2 protein in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody compounds from a library of such compounds in the presence of one or more CR2 ligands; and (c) detecting binding of one or more candidate non-antibody compounds to the immobilized CR2 protein or biologically-active fragment thereof, (d) isolating the one or more candidate non-antibody compounds bound to the immobilized CR2 protein or biologically-active fragment thereof, and (e) testing the one or more isolated candidate non-antibody compounds for the ability to competitively inhibit binding of one or more CR2 ligands to CR2 protein or biologically-active fragments thereof in a standard assay. In some embodiments, the CR2 ligand is EBV gp350 and the methods are used to identify non-antibody compounds that selectively bind to the extracellular domain of CR2 protein and competitively inhibit EBV binding to CR2 protein in a standard assay. In some embodiments, the non-antibody compounds selectively bind to SCRs 1 and 2 of CR2 protein, including the linker between SCRs 1 and 2 and competitively inhibit EBV binding to CR2 protein in a standard assay. In some embodiments, the non-antibody compounds selectively bind to SCRs 1 and 2 of the extracellular domain of CR2 protein and competitively inhibit EBV binding to CR2 protein in a standard assay. In some embodiments, the non-antibody compounds selectively bind to SCR 1 of the extracellular domain of CR2 protein and competitively inhibit EBV binding to CR2 protein in a standard assay. In some embodiments, the non-antibody compounds selectively bind to SCR 2 of the extracellular domain of CR2 protein and competitively inhibit EBV binding to CR2 protein in a standard assay. In some embodiments, the non-antibody compounds selectively bind to a binding site on CR2 protein comprising amino acid positions R13, S15, A22, R28, F35, R36, K41, D49, and D52 in a human CR2 protein sequence or their equivalent positions in a non-human CR2 protein sequence, and competitively inhibit EBV binding to CR2 protein in a standard assay. In some embodiments, the non-antibody compounds selectively bind to a binding site on CR2 protein comprising amino acid positions R13, A22, R28, F35, R36, K41, D49, D52, K57, E63, K67, Y68, R83, and R89 in a human CR2 protein sequence or their equivalent positions in a non-human CR2 protein sequence, and competitively inhibit EBV binding to CR2 protein in a standard assay. In some embodiments, the non-antibody compounds selectively bind to a binding site on CR2 protein comprising amino acid positions R13, A22, R28, K67, Y68, R83, G84, and S103 in a human CR2 protein sequence or their equivalent positions in a non-human CR2 protein sequence, and competitively inhibit EBV binding to CR2 protein in a standard assay.

In some embodiments, the invention provides methods of screening for ligand-specific non-antibody compounds that selectively bind to a CR2 protein, a ligand thereof, or both, wherein the ligand is a cell-bound proteolytic fragment of complement protein C3 (“C3 fragment”), and wherein the non-antibody compounds competitively inhibit the binding of one or more C3 fragments to a CR2 protein in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody compounds from a library of such compounds in the presence of one or more CR2 ligands; and (c) detecting binding of one or more candidate non-antibody compounds to the immobilized CR2 protein or biologically-active fragment thereof, (d) isolating the one or more candidate non-antibody compounds bound to the immobilized CR2 protein or biologically-active fragment thereof, and (e) testing the one or more isolated candidate non-antibody compounds for the ability to competitively inhibit binding of one or more CR2 ligands to CR2 protein or biologically-active fragments thereof in a standard assay. In some embodiments, the CR2 ligand is a C3 fragment (e.g., C3b, iC3d, C3dg, and C3d) and the methods are used to identify non-antibody compounds that selectively bind to a site on CR2 protein comprising SCRs 1 and 2 and the inter-SCR linker between SCRs 1 and 2 (SEQ ID NO: 4) of CR2 protein and competitively inhibit binding of one or more C3 fragments to CR2 protein in a standard assay. In some embodiments, the non-antibody compounds selectively bind to a site on CR2 protein comprising SCRs 1 and 2 and the inter-SCR linker between SCRs 1 and 2 (SEQ ID NO: 4) of CR2 protein and competitively inhibit binding of C3d to CR2 protein in a standard assay. In some embodiments, the non-antibody compounds selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, and competitively inhibit binding of one or more C3 fragments to CR2 protein in a standard assay. In some embodiments, the non-antibody compounds selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, and competitively inhibit binding of C3d to CR2 protein in a standard assay. In some embodiments, the non-antibody compounds selectively bind to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence, and competitively inhibit binding of one or more C3 fragments to CR2 protein in a standard assay. In some embodiments, the non-antibody compounds selectively bind to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence, and competitively inhibit binding of C3d to CR2 protein in a standard assay.

In some embodiments, the present invention provides methods of screening for ligand-specific non-antibody compounds that selectively bind to a CR2 protein, a ligand thereof, or both, wherein the ligand is IFN-α, and wherein the non-antibody compounds competitively inhibit the binding of IFN-α in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody compounds from a library of such compounds in the presence of one or more CR2 ligands; and (c) detecting binding of one or more candidate non-antibody compounds to the immobilized CR2 protein or biologically-active fragment thereof, (d) isolating the one or more candidate non-antibody compounds bound to the immobilized CR2 protein or biologically-active fragment thereof, and (e) testing the one or more isolated candidate non-antibody compounds for the ability to competitively inhibit binding of one or more CR2 ligands to CR2 protein or biologically-active fragments thereof in a standard assay. In some embodiments, the CR2 ligand is IFNα and the methods are used to identify non-antibody compounds that selectively bind to a site in SCRs 1 and 2 of CR2 protein and competitively inhibit IFN-α-binding to CR2 protein in a standard assay. In some embodiments, the non-antibody compounds bind to a site in SCRs 1 and 2 of CR2 protein and competitively inhibit IFN-α-binding to CR2 protein in a standard assay.

In some embodiments, the present invention provides methods of screening for ligand-specific non-antibody compounds that selectively bind to a CR2 protein, a ligand thereof, or both, wherein the ligand is FcεRII/CD23, and wherein the non-antibody compounds competitively inhibit the binding of FcεRII/CD23 in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody compounds from a library of such compounds in the presence of one or more CR2 ligands; and (c) detecting binding of one or more candidate non-antibody compounds to the immobilized CR2 protein or biologically-active fragment thereof, (d) isolating the one or more candidate non-antibody compounds bound to the immobilized CR2 protein or biologically-active fragment thereof, and (e) testing the one or more isolated candidate non-antibody compounds for the ability to competitively inhibit binding of one or more CR2 ligands to CR2 protein or biologically-active fragments thereof in a standard assay. In some embodiments, the CR2 ligand is FcεRII/CD23 and the methods are used to identify non-antibody compounds that selectively bind to CR2 protein and competitively inhibit FcεRII/CD23-binding to CR2 protein in a standard assay. In some embodiments, the non-antibody compounds bind to a site in SCRs 1 and 2 of CR2 protein and competitively inhibit FcεRII/CD23-binding to CR2 protein in a standard assay.

In some embodiments, the invention provides methods of screening for ligand-specific non-antibody polypeptides that selectively bind to a CR2 protein, a ligand thereof, or both, wherein the non-antibody polypeptides competitively inhibit ligand binding to a CR2 protein in a standard assay (e.g. a competitive ELISA). In some embodiments, the methods comprises the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody polypeptides from a library of such polypeptides, optionally in the presence of one or more CR2 ligands; and (c) detecting binding of one or more candidate non-antibody polypeptides to the immobilized CR2 protein or biologically-active fragment thereof, (d) isolating the one or more candidate non-antibody polypeptides bound to the immobilized CR2 protein or biologically-active fragment thereof, and (e) testing the one or more isolated candidate non-antibody polypeptides for the ability to competitively inhibit binding of one or more CR2 ligands to CR2 protein or biologically-active fragment thereof in a standard assay.

In some embodiments, the methods of screening for ligand-specific non-antibody polypeptides comprise random fragment library screening, structure-based in silico database mining, and phage display library screening. In some embodiment, the candidate non-antibody polypeptides can be synthesized by a solid-phase technique.

In some embodiments, the invention provides methods of screening for ligand-specific non-antibody polypeptides that selectively bind to a CR2 protein, a ligand thereof, or both, wherein the ligand is Epstein-Barr virus (EBV) and wherein the non-antibody polypeptides competitively inhibit ligand binding to a CR2 protein in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody polypeptides from a library of such polypeptides, optionally in the presence of one or more CR2 ligands; and (c) detecting binding of one or more candidate non-antibody polypeptides to the immobilized CR2 protein or biologically-active fragment thereof, (d) isolating the one or more candidate non-antibody polypeptides bound to the immobilized CR2 protein or biologically-active fragment thereof, and (e) testing the one or more isolated candidate non-antibody polypeptides for the ability to competitively inhibit binding of one or more CR2 ligands to CR2 protein or biologically-active fragment thereof in a standard assay. In some embodiments, the CR2 ligand is EBV gp350, and the non-antibody polypeptides competitively inhibit EBV binding to CR2 protein in a standard assay. In some embodiments, the non-antibody polypeptides competitively inhibit EBV gp350 binding to a CR2 SCR domain in a standard assay. In some embodiments, the non-antibody polypeptides selectively bind to a binding site on EBV gp350 comprising two N-terminal β-barrel domains (D1 and D2) and a linker sequence between D1 and D2 and competitively inhibit EBV binding to a CR2 protein in a standard assay. In some embodiments, the non-antibody polypeptides selectively bind to a binding site on EBV gp350 comprising amino acid positions E21, D22, E155, E201, D208, E210, and D296 in a wildtype EBV gp350 sequence or their equivalent positions in a variant EBV gp350 sequence, and competitively inhibit EBV binding to CR2 protein in a standard assay. In some embodiments, the non-antibody polypeptides selectively bind to a binding site on EBV gp350 comprising amino acid positions Y151, I160, and W162 in a wildtype EBV gp350 sequence or their equivalent positions in a variant EBV gp350 sequence and competitively inhibit EBV binding to CR2 protein in a standard assay. In some embodiments, the non-antibody polypeptides selectively bind to a binding site on EBV gp350 comprising amino acid positions E21, D22, E155, E201, D208, E210, D296, Y151, I160, and W162 in a wildtype EBV gp350 sequence or their equivalent positions in a variant EBV gp350 sequence and competitively inhibit EBV binding to CR2 protein in a standard assay.

In some embodiments, the invention provides methods of screening for ligand-specific non-antibody polypeptides that selectively bind to a CR2 protein and competitively inhibit EBV (e.g. EBV gp350) binding to a CR2 protein in a standard assay (e.g. a competitive ELISA). In some embodiments, the methods comprise the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody polypeptides from a library of such polypeptides, optionally in the presence of one or more CR2 ligands; and (c) detecting binding of one or more candidate non-antibody polypeptides to the immobilized CR2 protein or biologically-active fragment thereof, (d) isolating the one or more candidate non-antibody polypeptides bound to the immobilized CR2 protein or biologically-active fragment thereof, and (e) testing the one or more isolated candidate non-antibody polypeptides for the ability to competitively inhibit binding of one or more CR2 ligands to CR2 protein or biologically-active fragment thereof in a standard assay. In some embodiments, the non-antibody polypeptides selectively bind to the extracellular domain of CR2 protein and competitively inhibit EBV binding to CR2 protein in a standard assay. In some embodiments, the non-antibody polypeptides selectively bind to SCRs 1 and 2 of CR2 protein, including the linker between SCRs 1 and 2 and competitively inhibit EBV binding to CR2 protein in a standard assay. In some embodiments, the non-antibody polypeptides selectively bind to SCRs 1 and 2 of the extracellular domain of CR2 protein and competitively inhibit EBV binding to CR2 protein in a standard assay. In some embodiments, the non-antibody polypeptides selectively bind to SCR 1 of the extracellular domain of CR2 protein and competitively inhibit EBV binding to CR2 protein in a standard assay. In some embodiments, the non-antibody polypeptides selectively bind to SCR 2 of the extracellular domain of CR2 protein and competitively inhibit EBV binding to CR2 protein in a standard assay. In some embodiments, the non-antibody polypeptides selectively bind to a binding site on CR2 protein comprising amino acid positions R13, S15, A22, R28, F35, R36, K41, D49, and D52 in a human CR2 protein sequence or their equivalent positions in a non-human CR2 protein sequence, and competitively inhibit EBV binding to CR2 protein in a standard assay. In some embodiments, the non-antibody polypeptides selectively bind to a binding site on CR2 protein comprising amino acid positions R13, A22, R28, F35, R36, K41, D49, D52, K57, E63, K67, Y68, R83, and R89 in a human CR2 protein sequence or their equivalent positions in a non-human CR2 protein sequence, and competitively inhibit EBV binding to CR2 protein in a standard assay. In some embodiments, the non-antibody inhibitors selectively bind to a binding site on CR2 protein comprising amino acid positions R13, A22, R28, K67, Y68, R83, G84, and 5103 in a human CR2 protein sequence or their equivalent positions in a non-human CR2 protein sequence, and competitively inhibit EBV binding to CR2 protein in a standard assay

In some embodiments, the invention provides methods of screening for ligand-specific non-antibody polypeptides that selectively bind to a CR2 protein, a ligand thereof, or both, wherein the ligand is a cell-bound proteolytic fragment of complement protein C3 (“C3 fragment”), and wherein the non-antibody polypeptides competitively inhibit the binding of one or more C3 fragments to a CR2 protein in a standard assay (e.g. a competitive ELISA). In some embodiments, the methods comprises the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody polypeptides from a library of such polypeptides, optionally in the presence of one or more C3 fragments (e.g., C3b, iC3b, C3dg, and C3d); and (c) detecting binding of one or more candidate non-antibody polypeptides to the immobilized CR2 protein or biologically-active fragment thereof, (d) isolating the one or more candidate non-antibody polypeptides bound to the immobilized CR2 protein or biologically-active fragment thereof, and (e) testing the one or more isolated candidate non-antibody polypeptides for the ability to competitively inhibit binding of one or more C3 fragments (e.g., C3b, iC3b, C3dg, and C3d) to CR2 protein or biologically-active fragment thereof in a standard assay. In some embodiments, the non-antibody polypeptides selectively bind to a site on CR2 protein comprising SCRs 1 and 2 and the inter-SCR linker between SCRs 1 and 2 (SEQ ID NO: 4) of CR2 protein and competitively inhibit binding of one or more C3 fragments to CR2 protein in a standard assay. In some embodiments, the non-antibody polypeptides selectively bind to a site on CR2 protein comprising SCRs 1 and 2 and the inter-SCR linker between SCRs 1 and 2 (SEQ ID NO: 4) of CR2 protein and competitively inhibit binding of C3d to CR2 protein in a standard assay. In some embodiments, the non-antibody polypeptides selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, and competitively inhibit binding of one or more C3 fragments to CR2 protein in a standard assay. In some embodiments, the non-antibody polypeptides selectively bind to a binding site on CR2 protein comprising amino acid positions R13, Y16, R28, Y29, S32, T34, K48, D56, K57, Y68, R83, G84, N101, N105, S109, and R122 in a human CR2 protein sequence or their equivalent positions in a non-human CR2 protein sequence, and competitively inhibit binding of C3d to CR2 protein in a standard assay. In some embodiments, the non-antibody compounds selectively bind to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence, and competitively inhibit binding of one or more C3 fragments to CR2 protein in a standard assay. In some embodiments, the non-antibody compounds selectively bind to a binding site on CR2 protein comprising amino acid positions R13, Y16, T34, K48, D56, K57, Y68, R83, G84, N105, and S109 in a human CR2 protein sequence or their equivalent positions in a non-human CR2 protein sequence, and competitively inhibit binding of C3d to CR2 protein in a standard assay.

In some embodiments, the present invention provides methods of screening for ligand-specific non-antibody polypeptides that selectively bind to a CR2 protein, a ligand thereof, or both, wherein the ligand is IFN-α, and wherein the non-antibody polypeptides competitively inhibit the binding of IFN-α in a standard assay (e.g. a competitive ELISA). In some embodiments, the methods comprises the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody polypeptides from a library of such polypeptides, optionally in the presence of IFNα; and (c) detecting binding of one or more candidate non-antibody polypeptides to the immobilized CR2 protein or biologically-active fragment thereof, (d) isolating the one or more candidate non-antibody polypeptides bound to the immobilized CR2 protein or biologically-active fragment thereof, and (e) testing the one or more isolated candidate non-antibody polypeptides for the ability to competitively inhibit binding of IFNα to CR2 protein or biologically-active fragment thereof in a standard assay. In some embodiments, the non-antibody polypeptides bind to a site in SCRs 1 and 2 of CR2 protein and competitively inhibit IFN-α-binding to CR2 protein in a standard assay.

In some embodiments, the present invention provides methods of screening for ligand-specific non-antibody polypeptides that selectively bind to a CR2 protein, a ligand thereof, or both, wherein the ligand is FcεRII/CD23, and wherein the non-antibody polypeptides competitively inhibit the binding of FcεRII/CD23 in a standard assay (e.g. a competitive ELISA). In some embodiments, the methods comprises the steps of (a) providing a CR2 protein or biologically-active fragment thereof immobilized on a solid surface (e.g., a microtiter dish, magnetic beads, and the like); (b) contacting the immobilized CR2 protein or biologically-active fragment thereof with one or more candidate non-antibody polypeptides from a library of such polypeptides, optionally in the presence of FcεRII/CD23; and (c) detecting binding of one or more candidate non-antibody polypeptides to the immobilized CR2 protein or biologically-active fragment thereof, (d) isolating the one or more candidate non-antibody polypeptides bound to the immobilized CR2 protein or biologically-active fragment thereof, and (e) testing the one or more isolated candidate non-antibody polypeptides for the ability to competitively inhibit binding of FcεRII/CD23 to CR2 protein or biologically-active fragment thereof in a standard assay. In some embodiments, the non-antibody polypeptides bind to CR2-protein and competitively inhibit FcεRII/CD23-binding to CR2 protein in a standard assay. In some embodiments, the non-antibody polypeptides bind to a site in SCRs 1 and 2 of CR2 protein and competitively inhibit FcεRII/CD23-binding to CR2 protein in a standard assay.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g., amounts, concentrations, etc.) but some experimental errors and deviations should be allowed for. Unless otherwise indicated, molecular weight is average molecular weight, temperature is in degrees centigrade; and pressure is at or near atmospheric.

### Example 1

### MBP-CR2 SCR1-2-EBV gp350 Mutant ELISA

To characterize the CR2 SCR1-2-binding site on EBV gp350, we generated single-site substitutions targeting a glycan-free area of this molecule that was broadly implicated in CR2-ligand binding by previous crystal-driven multiple-site mutagenesis studies and, separately, by a peptide-mapping study. See Urquiza et al. (2005) J. Biol Chem 280:35598-605; and Young et al. (2007) J Biol Chem 282:36614-36625. Our alanine substitutions targeted residues within the two N-terminal β-barrel domains (D1, residues 4-153, and D2, residues 165-305, respectively) and the eleven-residue linker region that connects them (linker-1).

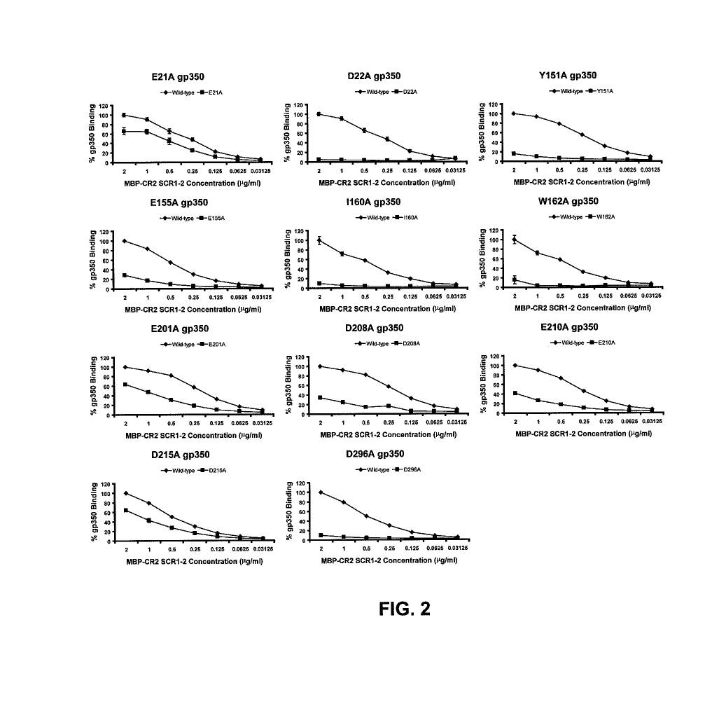

D1 of EBV gp350, E21A (in the crystal structure of EBV gp350 identified as Asp-21), D22A and Y151A showed significant (greater than 20%) decreases in CR2 SCR1-2 binding relative to that of wild-type EBV gp350, while D53A and E119A exhibited approximately wild-type-like levels of binding (FIG. 2; Table 1).

Within linker-1 of EBV gp350, we generated single-site mutations targeting those residues tentatively implicated as contributing to the EBV gp350-CR2 interaction in a previous multiple-site mutagenesis study. See Szakonyi et al. (2006) Nat Struct Mol Biol 13:996-1001. The current single-substitution mutagenesis data unexpectedly identified Ile-160 and Trp-162, as well as Glu155 (which was not identified previously) as key players in the ligation of MBP-CR2 SCR1-2 to EBV gp350, since all were unable to bind significant levels of CR2. On the other hand, Y159A and D163A mutant forms of EBV gp350 retained ability to bind wild-type-like levels of CR2 (Table 1).

Within D2, a double-site opposite charge substitution targeting residues Asp-208 and Glu-210 (D208R/E210R) was previously shown to inhibit binding of EBV gp350 to CR2, and also to disrupt the epitope for mAb 72A1. Id. These data were used to direct alanine-screening experiments targeting residues Glu-201, Asp-208, Glu-210, Glu-214, Asp-215, Glu-236 and Asp-296. Plate-bound forms of E201A, D208A, E210A, D215A, and D296A exhibited decreased ability to bind MBP-CR2 SCR1-2 (FIG. 2). The remaining D2-based mutants generated in the current study, E214A and E236A, which are spatially close to each other, exhibited no significant decreases in their capacity to bind MBP-CR2 SCR1-2 (Table 1).

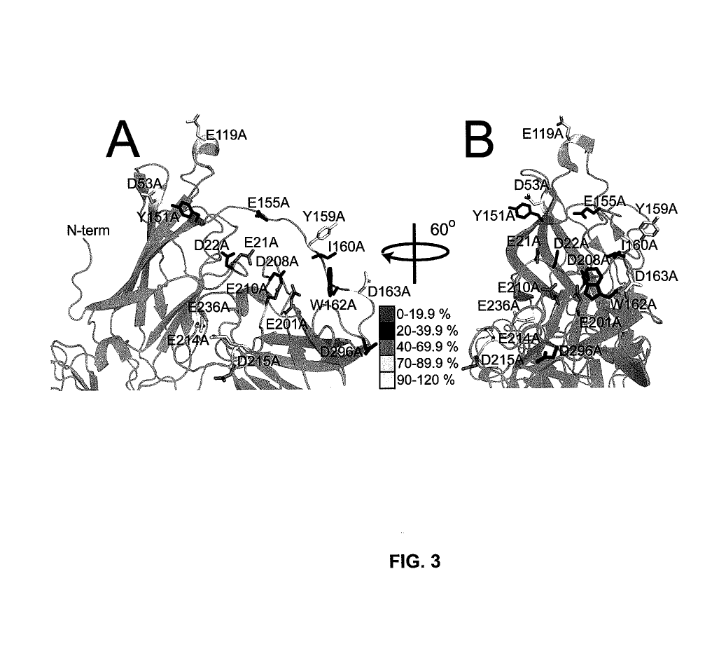

Mapping the results of the EBV gp350 mutant ELISA onto the crystal structure of EBV gp350 reveals a single binding surface for CR2 SCR1-2 on the rigid β-barrel domains comprising D1 and D2 (FIG. 3). This region is dominated by negatively charged residues. Further, additional contributions to the CR2 binding interaction are provided by residues within linker-1. These include some significant non-charge dependent interactions arising from Ile-160 and Trp-162, which were previously not considered relevant for binding.

Summary of wild-type CR2 binding to mutant forms of EBV gp350. Table 1 presents the data as the percentage CR2-binding of single-site mutant forms of EBV gp350 relative to those of wild-type EBV gp350, from both ELISA and cell-binding analyses. Key to weighting data: ++++>90%; +++89.9-70%; ++69.9-40%; +39.9-20%; −19.9-0%. MFI, Mean Fluorescent Intensity; S.E.M., standard error of the mean. Data are reported for concentrations of 2 μg/ml of MBP-CR2 SCR1-2, 5 μg/ml of EBV gp350 for the ELISA study and 5×10⁵ full-length CR2-transfected K562 cells, 5 μg/ml of EBV gp350-biotin for the cell-binding study. Mutants indicated in bold demonstrate a greater than 20% decrease in binding affinities relative to wild-type CR2, in both ELISA and the cell-binding assays. Residues (wild-type) which have been shaded have been defined as ‘active’ for the purposes of a molecular soft docking study.

TABLE 1

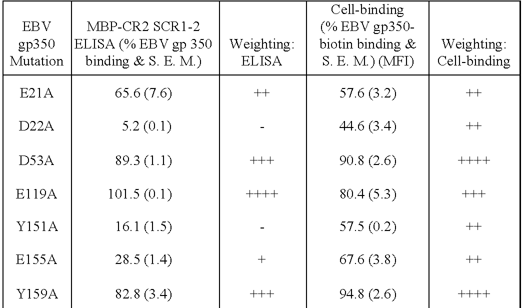

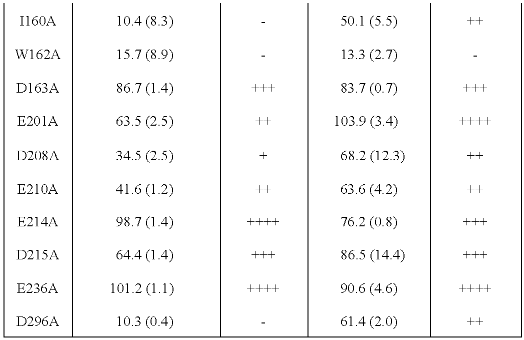

### Example 2

### EBV gp 350-Biotin-K562 Erythroleukemia Cell-Binding Assay

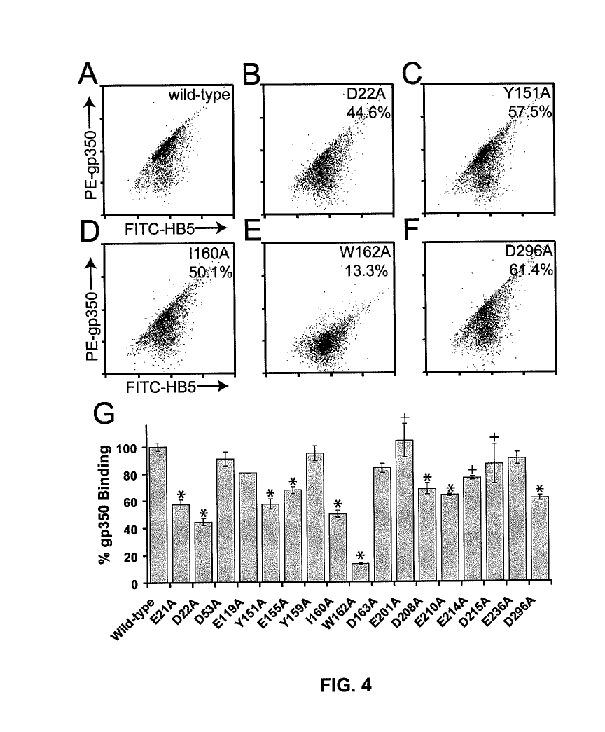

K562 erythroleukemia cells expressing wild-type CR2 were initially incubated with the non-inhibitory anti-CR2 mAb HB5, and subsequently FITC-ylated by incubation with FITC-conjugated goat anti-mouse anti-IgG. They were then assessed for their capacity to bind PE-conjugated wild-type or mutant forms of EBV gp350-biotin using flow cytometry. For the most part the data obtained were in excellent agreement with the MBP-CR2 SCR1-2-EBV gp350 ELISA analysis described above (FIG. 4; Table 1). For example, D21A, E22A, Y151A, E155A, I160A, W162A, D208A, E210A and D296A mutant forms of EBV gp350-biotin all exhibited decreases (>20%) in their capacity to bind to full-length CR2 SCR1-15, although the decrease in binding was less marked than that observed by ELISA. In particular, within the linker-1 region, E155A, I160A, and W162A were unexpectedly identified to critically affect CR2 binding. Further, consistent with the ELISA analysis, D53A, E119A, Y159A, D163A, and E236A mutant forms of EBV gp350-biotin demonstrated wild-type-like levels of binding. Three of the seventeen mutants tested, E201A, E214A and D215A were exceptions in that they displayed differing levels of binding to those observed in the ELISA analysis: E201A and D215A both retained wild-type-like levels of binding (103.4% and 86.5%, respectively), while E214A (76.2%) exhibited only a slight variation in binding from the wild-type-like binding curve seen in the ELISA.

### Example 3

### MBP-CR2 SCR1-2 Mutant-EBV gp350 ELISA

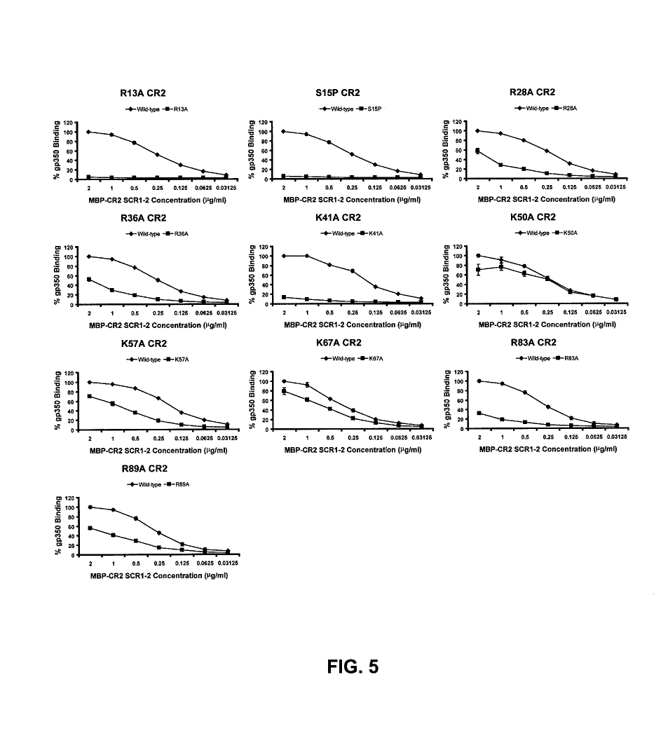

Mutations within the two N-terminal SCR domains of CR2 were selected on the basis that SCR1 of CR2 is characterized by a large number of positively charged residues, and the interaction between CR2 and EBV gp350 has been demonstrated to be charge-dependent in nature. See Martin et al. (1991) J Exp Med 174:1299-311; Young et al. (2007) J Biol Chem 282:36614-36625; Guthridge et al. (2001) Biochemistry 40:5931-41; and Sarrias et al. (2001) J Immunol 167:1490-9. In the present application, mutations directed at residues Arg-13 (R13A), Arg-28 (R28A), Arg-36 (R36A), Lys-41 (K41A) and Lys-57 (K57A), resulted in significantly decreased capacity of recombinant MBP-CR2 SCR1-2 to bind EBV gp350 (FIG. 5; Table 2). In addition, a single mutation targeting Ser-15 (S15P) within the first inter-cysteine region of SCR1 (chosen on the basis of comparison with the mouse orthologue of CR2) also resulted in a major reduction in EBV gp350 binding. Additional mutations targeting Leu-10 (L10A), Asn-11 (N11A), Tyr-16 (Y16A), Ser-32 (S32A), Thr-34 (T34A) and Lys-50 (K50A) all failed to inhibit EBV gp350 binding (Table 2). These data show an SCR1-dominant contribution to EBV gp350 attachment, and also expand the surface area of this domain that has been mutated and analyzed. One substitution, Y16A, exhibited surprisingly differing levels of EBV gp350 binding to that previously observed in the cell-binding study (66.7% in the cell-binding study and 95.1% in the ELISA; Table 2). Young et al. (2007) J Biol Chem 282:36614-36625.

Three residues were targeted for mutagenesis within the eight-residue (63-EYFNKYSS-70) linker region connecting SCR1 and SCR2: Tyr-64, Lys-67 and Tyr-68 were each substituted with an alanine residue (Y64A, K67A and Y68A). Only the K67A substitution demonstrated any significant effect on EBV gp350 binding (FIG. 3) with an approximately 20% decrease (FIG. 5).

Within SCR2 a total of four residues were selected for mutagenesis screening: Arg-83 (R83A), Thr-86 (T86A), Arg-89 (R89A) and Met-117 (M117A). Of the four alanine substitutions generated in SCR2, only the R83A and R89A mutants exhibited decreased binding affinities for EBV gp350 (FIG. 5).

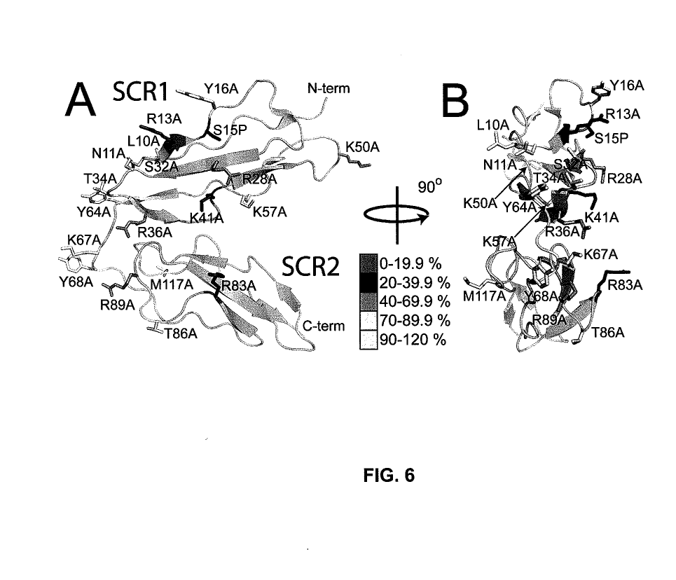

When mapped onto the crystal structure of CR2 SCR1-2 the data obtained using the MBP-CR2 SCR1-2 mutants delineates a binding interface that consists of a contiguous positively charged surface spread over SCR1 and SCR2, with two hot spots centred around Arg-83 and Arg-89 on SCR2 and Arg-13, Ser-15, Arg-28, Arg-36 and Lys-41 on SCR1 (FIG. 6) that are essential for the attachment of EBV gp350.

Summary of wild-type or mutant MBP-CR2 SCR1-2-EBV gp350 binding ELISA data. Table 2 presents ELISA data given as percentage EBV gp350-binding to mutant CR2 relative to wild-type CR2. Key to weighting data: ++++>90%; +++89.9-70%; ++69.9-40%; +39.9-20%; −19.9-0%. * Binding data and weighting obtained from previously reported cell-binding analysis of the CR2-EBV gp350 binding interaction (46). n/a, not applicable; S.E.M., standard error of the mean. Mutants indicated in bold demonstrate a greater than 20% decrease in binding affinities relative to wild-type EBV gp350, in both ELISA and the cell-binding assays (where available). ^{Ω} while the K50A mutant demonstrates a reduction in binding at the protein concentrations described in this Table, at other values recorded for both the ELISA and cell-binding analyses, K50A-binding approximates to that of wild-type CR2 (FIG. 5 and (1, 46)). Residues (wild-type) which have been shaded have been defined as ‘active’ to drive a molecular soft docking study.

TABLE 2

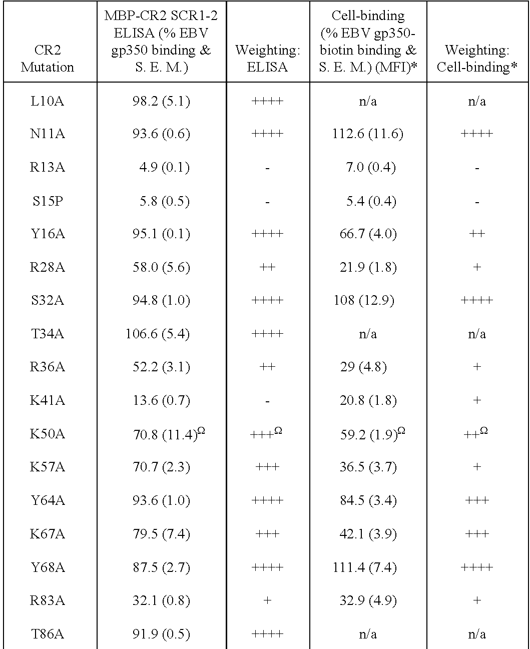

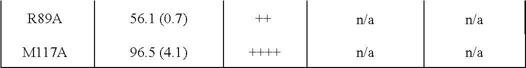

### Example 4

### MBP-CR2 SCR1-2-171 mAb and MBP-CR2 SCR1-2-629 mAb ELISA

The excellent correlation observed between our cell-binding studies and the ELISA analyses provides proof-of-principle that the MBP-CR2 SCR1-2 ELISA is an effective means of characterizing CR2-ligand interactions. We therefore utilized our nineteen MBP-CR2 SCR1-2 mutants to describe the structured epitope for the anti-CR2 SCR1-2 monoclonal antibody 171, which inhibits the interaction with EBV gp350 as well as all other known CR2 ligands. See Asokan et al. (2006) J Immunol 177:383-94; Guthridge et al. (2001) J Immunol 167:5758-66; Young et al. (2007) J Biol Chem 282:36614-36625. ELISA revealed that single-site mutants targeting a patch of residues located within the first inter-cysteine region of SCR1 of CR2, comprising Asn-11, Arg-13, Ser-32, Thr-34 and Arg-36 (N11A, R13A, S32A, T34A and R36A), and also a single residue within the linker region connecting SCR1 and SCR2, Tyr-64 (Y64A), disrupted the epitope for mAb 171 (FIG. 1C; Table 3). This epitope overlaps extensively with the binding surface on CR2 described above for EBV gp350, and also with the previously described binding sites for C3dg and C3d. Hannan et al. (2005) J Mol Biol 346:845-58; and Young et al. (2007) J Biol Chem 282:36614-36625. By contrast, none of these MBP-CR2 SCR1-2 mutants were found to have any deleterious effects on the binding of a non-inhibitory anti-CR2 SCR1-2 mAb, 629. This suggests that the epitope for mAb 629 is located on a surface discrete from the ligand-binding sites described above for EBV gp350, and also for the previously characterized C3 fragments, C3dg and C3d (Table 3). A single alanine substitution targeting Tyr-16 (Y16A) demonstrated increased binding (˜150%) to mAb 629 relative to that of wild-type MBP-CR2 SCR1-2.

Summary of wild-type or mutant MBP-CR2 SCR1-2 binding to the anti-CR2 SCR1-2 mAbs, 171 and 629. Weighting used as described above for Table 1 and Table 2. Mutants indicated in bold demonstrate a greater than 20% decrease in binding affinities relative to wild-type CR2.

TABLE 3

| | 171 mAb | | | 629 mAb | | |
|---|---|---|---|---|---|---|
| | % 171 mAb | | | % 629 mAb | | |
| CR2 | Binding & | | | Bindng & | | |
| Mutation | S.E.M | | Weighting | S.E.M | | Weighting |
| L10A | 94.7 | (3.2) | ++++ | 113.2 | (6.4) | ++++ |
| N11A | 24.1 | (1.4) | + | 87.5 | (2.1) | +++ |
| R13A | 15.6 | (2.1) | − | 108.1 | (0.8) | ++++ |
| S15P | 108.5 | (1.6) | ++++ | 91.7 | (7.3) | ++++ |
| Y16A | 81.4 | (3.7) | +++ | 154.4 | (4.0) | ++++ |
| R28A | 103.5 | (1.7) | ++++ | 103.1 | (1.8) | ++++ |
| S32A | 77.5 | (8.9) | +++ | 117.8 | (3.4) | ++++ |
| T34A | 12.4 | (1.4) | − | 84.9 | (5.8) | +++ |
| R36A | 19.8 | (1.7) | − | 113.9 | (1.5) | ++++ |
| K41A | 97.3 | (1.7) | ++++ | 97.5 | (0.8) | ++++ |
| K50A | 94.4 | (3.5) | ++++ | 87.5 | (3.2) | +++ |
| K57A | 91.9 | (0.8) | ++++ | 85.5 | (0.7) | +++ |
| Y64A | 5.8 | (0.1) | − | 82.0 | (1.5) | +++ |
| K67A | 87.7 | (3.2) | +++ | 107.2 | (4.5) | ++++ |
| Y68A | 92.1 | (4.8) | ++++ | 102.3 | (4.1) | ++++ |
| R83A | 96.9 | (2.4) | ++++ | 94.4 | (3.9) | ++++ |
| T86A | 89.6 | (5.2) | +++ | 85.6 | (3.3) | +++ |
| R89A | 93.9 | (7.8) | ++++ | 87.7 | (2.1) | +++ |
| M117A | 97.2 | (4.1) | ++++ | 113.8 | (2.9) | ++++ |

### Example 5

### HADDOCK-Derived CR2-EBV gp350 Model

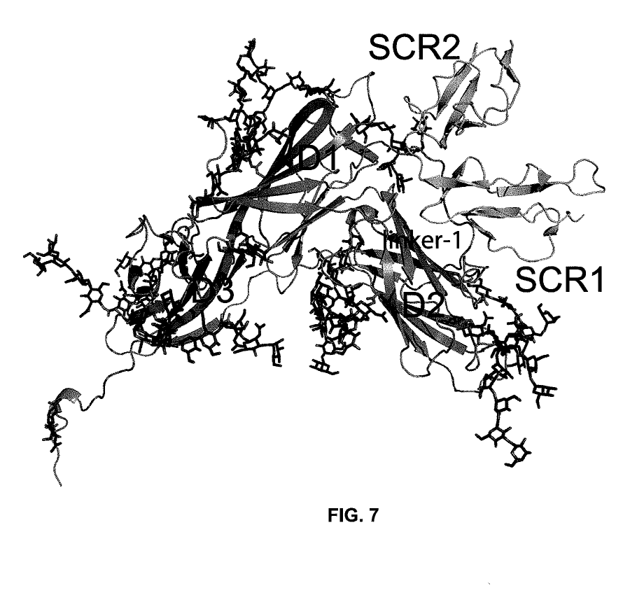

The mutagenesis data for EBV gp350 and CR2 obtained in the current study were used to generate a series of ambiguous interaction restraints (AIRs) that were subsequently employed by the soft docking program HADDOCK (Dominguez et al. (2003) J Am Chem Soc 125:1731-7) to derive a model of the CR2 SCR1-2-EBV gp350 complex. Solvent-accessible (50% or greater) residues on CR2 and EBV gp350, mutation of which disrupts complex formation according to both our ELISA and cell-binding assays, were designated as “active” residues for the docking calculations (Tables 1 and 2). Starting from the randomly oriented structures of CR2 and EBV gp350, active residues were used by HADDOCK to direct the docking process towards the most plausible complex of CR2 and EBV gp350 which also satisfied the data previously recorded (FIG. 7). A cluster analysis of the resulting 100 water-refined structures, employing a 1.5 Å cut-off limit, revealed that 99 of these models fell into a single cluster. For the 100 water-refined structures analyzed, the backbone rmsd at the intermolecular interface for all structures was 1.5 Å (1.9 Å for all backbone atoms in all structures). The average buried surface area for the 100 water-refined structures was 2510±192 Å². The structures calculated are in good agreement with experimental evidence as determined by an average of 0.04 AIR restraint violations >0.5 Å per structure. Our models of the EBV gp350-CR2-SCR1-2 complex are also highly structurally uniform and are in broad agreement with results generated using an incomplete set of AIR restraints (18). The backbone interface rmsd values obtained for the CR2-EBV gp350 complex are consistent with other high-scoring HADDOCK-derived models, as reported in the fourth and fifth rounds of the ‘Critical Assessment of Prediction of Interactions’ (CAPRI) which ranged from 0.3-2.0 Å (44). Moreover, the CR2 SCR1-2 binding site on EBV gp350 is highly plausible, being located within a negatively charged region on the surface of this molecule that is devoid of the glycan moieties that cover much of the surface.

### Experimental Procedures for Examples 1-5

Production of the anti-CR2 monoclonal antibodies 171 mAb and 629 mAb. Anti-CR2 mAbs, 171 and 629, were obtained from the spent culture medium of hybridoma cells grown in RPMI 1640 supplemented with 2 mM L-glutamine, 100 IU Penicillin, 100 ug/mL Streptomycin, and 10% fetal bovine serum. Antibodies were purified by affinity chromatography using Protein G Sepharose 4 Fast Flow resin (GE Healthcare Biosciences Corp) according to the manufacturer's instructions. Purified 171 and 629 mAb were subsequently exchanged into PBS, pH 7.4 (containing 136.9 mM NaCl, 8.1 mM Na₂HPO₄, 2.7 mM KCl, 1.5 mM KH₂PO₄), and finally concentrated to give a stock solution containing 1 mg/ml of antibody as determined by UV-visible spectrophotometry and stored at −20° C. until required.

Expression of wild-type and mutant MBP-CR2 SCR1-2 recombinant proteins in Escherichia coli. DNA corresponding to residues 1-133 of wild-type CR2 (SCR1-2) was PCR amplified and then ligated into the prokaryotic expression vector pMAL-p2x (New England Biolabs) which encodes a maltose binding protein (MBP) tag at the 5′ prime end of the inserted DNA, as previously described. Plasmid DNA was subsequently transformed into E. coli BL21 cells and wild-type recombinant MBP-CR2 SCR1-2 was produced according to earlier protocols. See Young et al. (2007) J Biol Chem 282:36614-36625. Recombinant wild-type MBP-CR2 SCR1-2 protein was expressed from overnight cultures induced with IPTG. Cultures were then harvested by centrifugation and resulting pellets were resuspended in a column buffer comprising 20 mM Tris-HCl, pH 7.4, 0.2 M NaCl, 1 mM EDTA, and lysed by sonication. The lysate was clarified by centrifugation and then purified by successive affinity (amylose resin; New England Biolabs) and size-exclusion (Hiload S200 26/60; GE Biosciences) chromatography stages

Recombinant L10A, N11A, R13A, S15P, Y16A, R28A, S32A, T34A, R36A, K41A, K50A, K57A, Y64A, K67A, Y68A, R83A, T86A, R89A and M117A forms of CR2 SCR1-2 DNA were produced from wild-type MBP-CR2 SCR1-2 DNA utilizing a Quickchange site-directed mutagenesis kit (Stratagene) according to the manufacturer's instructions. Plasmid DNA containing the mutant CR2 SCR1-2 insert was then transformed into E. coli BL21 and recombinant mutant CR2 SCR1-2 proteins were expressed and purified as described above.

Expression of recombinant EBV gp350 proteins. EBV genomic DNA was extracted from previously obtained cell supernatants of the marmoset B95-8 leukocyte cell-line (American type Culture Collection; ATCC) using a QIAamp UltraSens virus kit (Qiagen) as described previously (46). DNA corresponding to residues 1-470 of EBV gp350/220 and a fragment of the E. coli biotin carboxyl carrier protein (BCCP) corresponding to residues 70-156 were separately PCR amplified. PCR fragments were then ligated into the pSecTag2/Hygro B eukaryotic expression vector (Invitrogen), which encodes a myc epitope and a hexa-histidine tag at the 3′-end of the inserted DNA. Plasmid DNA was subsequently transfected into human embryonic kidney 293f freestyle cells (Invitrogen) for soluble expression of recombinant EBV gp350 into the media. Resulting recombinant EBV gp350 was concentrated and concurrently exchanged into a sodium phosphate column buffer for purification purposes. Recombinant EBV gp350 protein was purified utilizing successive immobilized metal-affinity and size-exclusion chromatography steps. After being purified, EBV gp350 was either aliquoted and then frozen at −70° C. until required for ELISA analysis, or else biotinylated (EBV gp350-biotin) using biotin ligase (Avidity). The resulting EBV gp350-biotin was conjugated to phycoerythrin (PE)-NeutrAvidin (Molecular Probes) generating fluorochrome-tagged EBV gp350 monomers for dual-color flow cytometric binding analysis.

Mutant forms of EBV gp350 were generated targeting a number of residues located within domain 1 (D1) and domain 2 (D2) of EBV gp350 and also the eleven residue linker region (L-1) connecting the two domains. Residues Glu-21, Asp-22, Asp-53, Glu-119, Tyr-151, Glu-155, Tyr-159, Ile-160, Trp-162, Asp-163, Glu-201, Asp-208, Glu-210, Glu-214, Asp-215, Glu-236 and Glu-296 were selected for alanine-screening studies to delineate the CR2 binding site on the EBV gp350 molecule and also to characterize the nature of the interaction between the two molecules. Single-site mutant forms of recombinant EBV gp350 were produced from wild-type B95-8 EBV gp350 DNA utilizing a Quikchange site-directed mutagenesis kit (Stratagene) according to the manufacturer's instructions. Recombinant mutant EBV gp350 proteins were expressed and purified as described above for wild-type.

CR2-EBV gp350, CR2-171 mAb and CR2-629 mAb ELISAs. Plates were coated overnight at 4° C. with 5 μg/ml of EBV gp350, 171 mAb or 629 mAb, in 20 mM sodium bicarbonate buffer, pH 8.8. After coating, the plates were blocked using 0.1% BSA in a PBS solution, pH 7.4 for one hour at room temperature. The plates were then washed, and incubated with either wild-type or mutant MBP-CR2 SCR1-2, at concentrations ranging from 0.03125-2.0 μg/ml in PBS for one hour at room temperature. After further washing, wild-type or mutant MBP-CR2 binding was detected using commercially available horseradish peroxidase-conjugated anti-MBP mAb (New England Biolabs) according to the manufacturer's instructions.

Flow Cytometry. Flow cytometric experiments were carried out using K562 erythroleukemia cells transfected with full-length wild-type CR2 as described previously. See Hannan et al. (2005) J Mol Biol 346:845-58; and Young et al. (2007) J Biol Chem 282:36614-36625. Binding analyses were carried out using wild-type or mutant forms of EBV gp350-biotin. For each condition, 5×10⁵ human CR2-transfected K562 cells were first incubated with anti-CR2 HB5 mAb at 1 μg/ml on ice for one hour. Subsequently the HB5-coated cells were incubated with fluorescein isothiocyanate (FITC)-conjugated goat anti-mouse polyclonal antibody (BD Biosciences) at 1 μg/ml on ice for 30 minutes. The primary epitope for HB5 has been identified within the N-terminal SCR3-4 domains of CR2 and accordingly does not interfere with ligand binding. During this incubation, 100 μl of EBV gp350-biotin monomers in PBS/0.1% BSA/0.01% sodium azide were prepared for each condition by adding 0.5 μg of recombinant wild-type or mutant EBV gp350-biotin and 0.4 μg PE-conjugated NeutrAvidin (Molecular Probes) and incubating at room temperature for 30 minutes. Following washing of the FITC stained K562 cells, 100 μl of monomeric PE-conjugated EBV gp350-biotin were added to each sample of cells and incubated for 30 minutes on ice. After washing, the cells were fixed and analyzed by multi-color flow cytometry in the University of Colorado Cancer Center Flow Cytometry Core Facility (Denver, Colo.). Cells were analyzed as previously described, gating on either whole cell populations and/or the intermediate 25% of CR2-expressing cells (FITC-positive). Wild-type or mutant EBV gp350-biotin binding was determined by PE mean channel fluorescence. A minimum of three separate experiments was carried out for each mutation.

Generation by HADDOCK of a CR2-EBV gp350 model. Mutagenesis data were used to derive a series of models of the CR2-EBV gp350 complex utilizing version 1.3 of the soft-docking program HADDOCK. See Dominguez et al. (2003) J Am Chem Soc 125:1731-7. This employs a knowledge-based approach by which the program utilizes experimentally-derived data, in conjunction with the available structures, to drive the docking of two macromolecules. Residues which have been experimentally implicated in a binding interaction and which are therefore likely to form part of the interface between two molecules are designated as “active”. In this case, active residues for each molecule were defined as those residues that our mutagenesis data have identified as playing a significant role in the binding interaction between CR2 and EBV (Young et al. (2007) J Biol Chem 282:36614-36625), and which possessed a main-chain or side-chain solvent accessibility of 50% or greater (as determined by the program NACCESS (S. Hubbard and J. Thornton). Selected active residues for CR2 were: Arg-13, Ser-15, Arg-28, Lys-41, Lys-67, and Arg-83. For EBV gp350 binding, selected active residues were: Glu-21, Tyr-151, Glu-155, Ile-160, Trp-162, Asp-208, Glu-210, and Asp-296. Also used in the HADDOCK calculations are “passive” residues—defined as those residues in close proximity to “active” residues with high main-chain or side-chain solvent accessibility. This information was introduced in the form of ambiguous interaction restraints (AIRs) which are ambiguous distances that are defined between any atom of the active residues of one protein and all atoms of the active and passive residues on the binding partner. Dominguez et al. (2003) J Am Chem Soc 125:1731-7. Standard analyses were performed by HADDOCK on 100 water-refined structures from an initial 2,000 structures calculated, and including an analysis of the energy contributions from buried surface area and electrostatic interactions.

### Example 6

### Identification of Selective Inhibitory Peptides

Linear and disulfide constrained consensus peptides were identified by phage display library methods. Phage were targeted to recombinant CR2 SCR1-2 and binding phage were competed off with each ligand to attempt to identify peptides that would block binding of one but the not the other ligand. After three subsequent panning rounds with increasing detergent concentrations, selected phage were sequenced. A consensus peptide sequence was determined and the corresponding peptide was synthesized by conventional solid-phase techniques. See Merrifield (1969) Adv Enzymol relat Areas Mol Biol 32:221-96. Peptides were identified that blocked recombinant C3d and Epstein Barr Viral coat glyco-protein EBV gp350 (see Table 4).

Similarly, inhibitory synthetic molecules targeting CR2-C3d and CR2-gp350 complex formation will also be identified by random fragment library screening or structure-based and driven by in silico database mining.

### Example 7

### Peptide Competition ELISA

Each peptide was tested, in increasing concentration against its respective ligand. Percentage inhibition was calculated and plotted against peptide concentration to illustrate an inhibition curve. From this data an IC50 was calculated and listed in Table 4.

Initial inhibitory peptides were modified to attempt to construct more efficacious inhibitors. First, peptides were shortened systematically to remove unnecessary amino acids. Last, the amino acid sequence of each peptide was altered to contain an alanine residue for each amino acid thought to play an inhibitory role in the sequence.

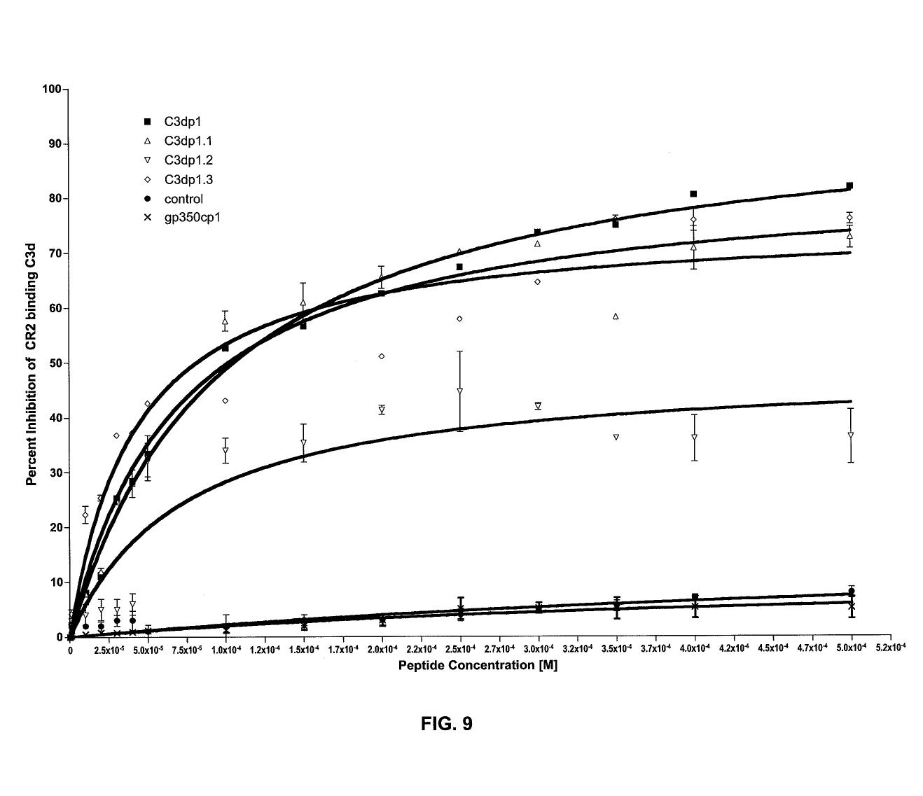

Peptides identified against C3d showed varying levels of inhibition (Table 4 and FIG. 9). Specifically, C3dp1 (sequence: APQHLSSQYSRT) showed an IC50 of 100 μM. This peptide served as a starting point to determine the residues required for inhibition. First, the His residue at position four was substituted for an Ala residue. This peptide, C3dp1.1 (sequence: APQALSSQYSRT) showed an IC50 of 70 μM, a slight increase in inhibition. Further, to determine whether or not this peptide was charge dependent or structurally dependent, the amino acid sequence was reversed (C3dp1.2; sequence: TRSYQSSLHQPA), this peptide also showed a slight increase in inhibition (70 μM). The parent peptide was next shortened to include only the first eight amino acids (C3dp1.3; sequence: APQHLSSQ), this peptide showed the best inhibition at 40 μM. In order to validate the above peptides, a control peptide was generated. The control peptide that was identified was very similar to C3dp1.3, differing only at position three where the Gln residue was substituted for an Ala residue (control; sequence APAHLSSQ). This single amino acid change changed the IC50 from 40 to 700 μM, thus was deemed a good control peptide. The CR2-C3d interaction is very salt, thus competition ELISAs were carried out in ⅓×PBS to study the slight interactions that contribute to the overall interaction.

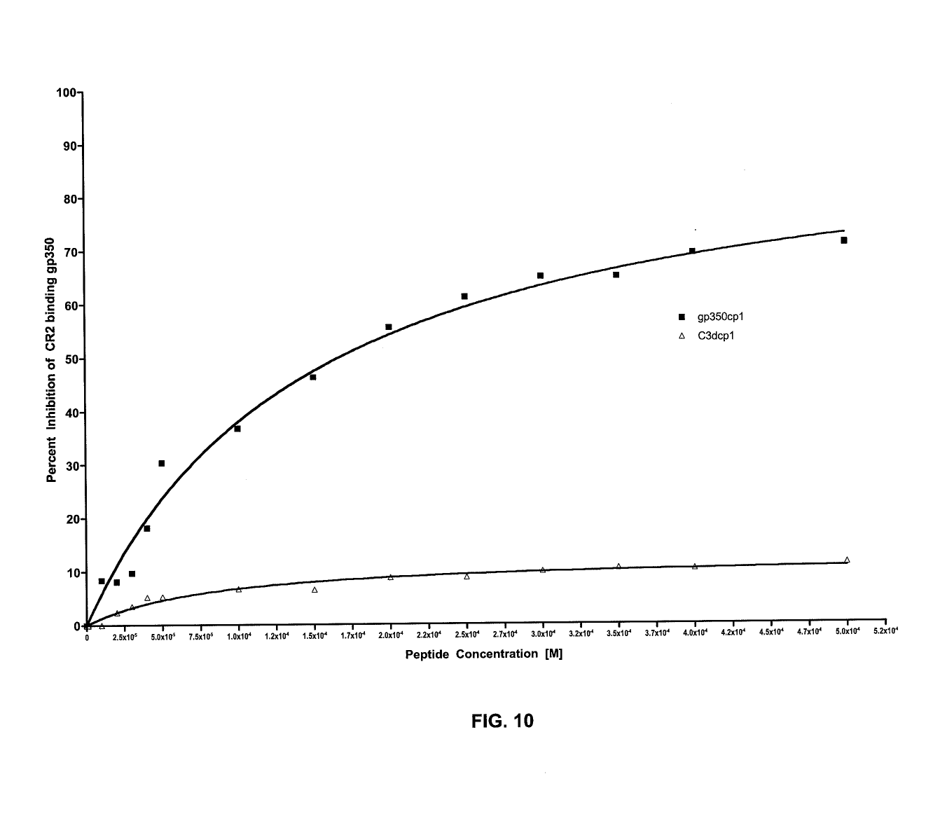

The peptide determined against EBV gp350 showed an IC50 of 150 μM (Table 4 and FIG. 10). The CR2-EBV gp350 interaction has been shown to be a tighter binding interaction (Asokan et al. (2006) J Immunol 177:383-94), thus making inhibition via a peptide very difficult. The disulfide constrained peptide EBV gp350 cp1 was found to inhibit the CR2-EBV gp350 interaction in physiological conditions (PBS).

Of particular importance the inhibitory peptides presented here are specific to each interaction. Thus peptides derived against the CR2-C3d inhibit only the CR2-C3d interaction. And conversely the peptide determined against the CR2-EBV gp350 interaction inhibits only the CR2-EBV gp350 interaction (FIGS. 9 and 10).

Identified inhibitory peptides against CR2-ligand interactions. Shown are inhibitory peptides against CR2-C3d and CR2-EBV gp350 interactions identified by phage display, truncation and alanine scanning. EBV gp350 cp1 peptide is disulfide constrained by making a cysteine bridge between residues 1 and 9.

TABLE 4

| Peptide | | IC50 |
|---|---|---|
| Name | Peptide Sequence | (μM) |
| C3dp1 | APQHLSSQYSRT | 100 |
| C3dp1.1 | APQALSSQYSRT | 70 |
| C3dp1.3 | APQHLSSQ | 40 |
| control | APAHLSSQ | 700 |
| C3dp2 | ISTSNPRHHSTA | 450 |
| C3dp2.1 | ISTSNPRAASTA | 150 |
| C3dp2.2 | ISTSNPRH | 190 |
| C3dp2.3 | IATANPRHHSTA | 160 |
| C3dcp1 | CDPKNHVHC | 640 |
| C3dcp1.1 | DPKNHVH | 95 |
| gp350cp1 | CSEGSLKGC | 150 |

### Example 8

### NMR Chemical Shift Mapping

To study the structural interface of the CR2-C3d interaction NMR was employed. ¹H, ¹⁵N assignments were made using a combination of 3D experiments (Cavanagh et al. (2007), Protein NMR spectroscopy: principles and practice. 2^{nd} ed. Amsterdam, Boston: Academic Press. xxv, p. 885) acquired using a ¹³C, ¹⁵N and a ¹⁵N labeled CR2 SCR1-2 sample. Backbone sequential assignments were made using CBCA(CO)NH, HNCACB and 3D ¹⁵N-edited NOESY-HSQC. Spectra were processed using nmrPipe (Delaglio et al. (1995) J Biomol NMR 6:277-93) and analyzed using ccpNMR (Vranken et al. (2005) Proteins 59:687-96). The ¹H, ¹⁵N-TROSY-HSQC of CR2 SCR1-2 was assigned to 90%.

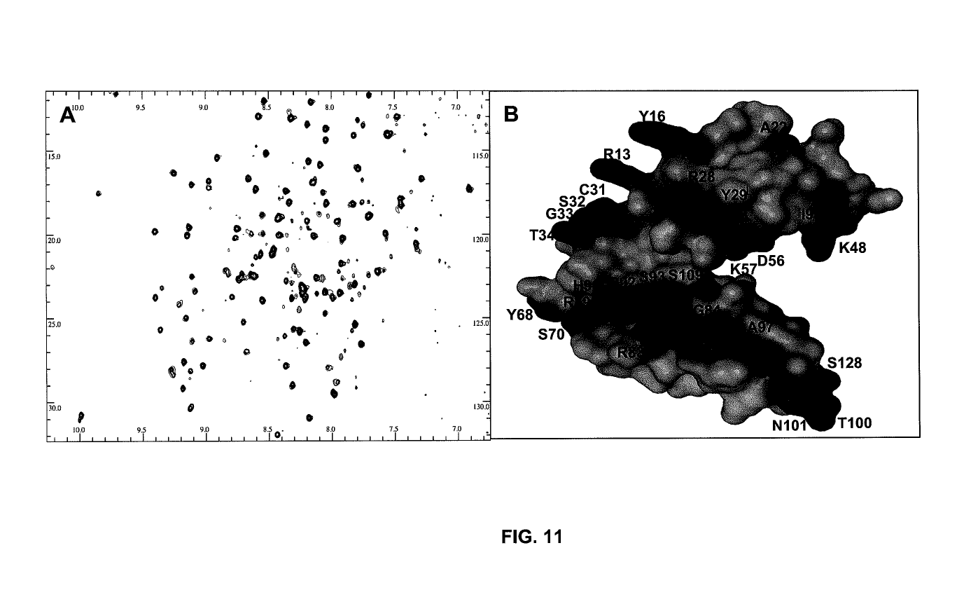

Full length unlabeled C3d was titrated into a uniformly ¹⁵N labeled CR2 SCR1-2 sample and the ¹H-¹⁵N chemical shifts were monitored (FIG. 11A). Those residues showing the most chemical shift changes were residues R13, Y16, R28, Y29, S32, T34, K48, D56, K57, Y68, R83, G84, N101, N105, S109 and R122. These residues encompass both SCR1 and SCR2 domains and the intra-SCR linker region (FIG. 11B). These residues suggest two distinct non-contiguous interaction faces on CR2 SCR1-2 indicating that the linker region allows SCR1 and SCR2 to be oriented such that each intimately contacts C3d.

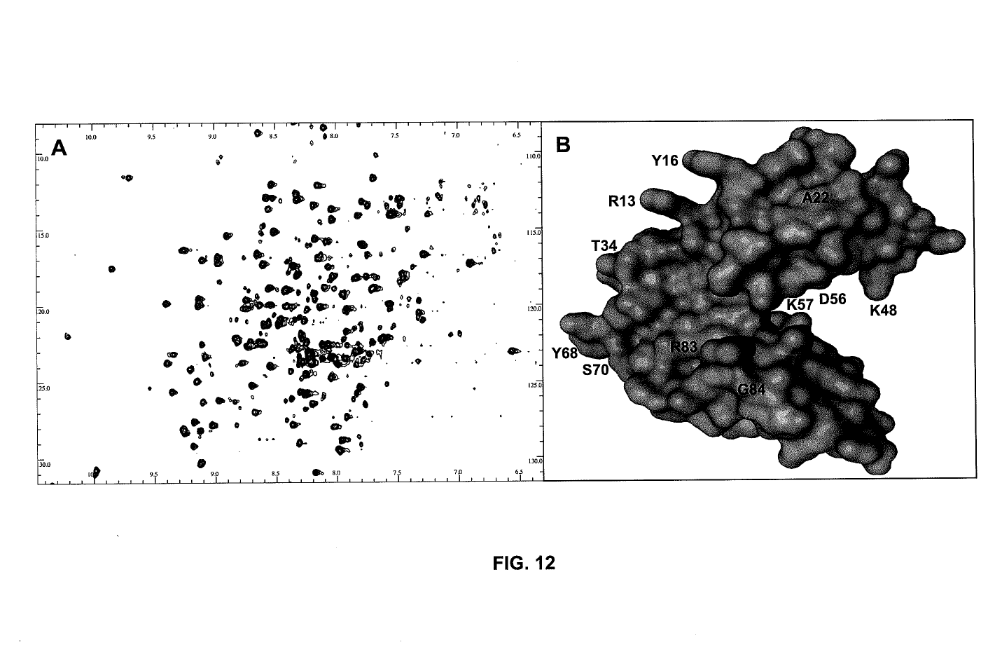

To further validate the specificity of the inhibitory peptides, C3dp1 was titrated into a uniformly ¹⁵N labeled CR2 SCR1-2 sample and the ¹H-¹⁵N chemical shifts were monitored (FIG. 12A). The residues showing the most chemical shift changes were residues R13, Y16, T34, K48, D56, K57, Y68, R83, G84, N105 and S109. Again these residues encompass both SCR domains and linker region, as well as overlapping with the CR2-C3d chemical shifts (FIG. 11A). The interacting residues were mapped onto the surface of the x-ray structure (FIG. 12B). These data are suggestive that the peptide is specifically interacting with the same residues as the full-length ligand, an important finding since some ligands are not amiable to NMR titration experiments because of the size limitations that come with using NMR. See Cavanagh et al. (2007), Protein NMR spectroscopy: principles and practice. 2^{nd} ed. Amsterdam, Boston: Academic Press. xxv, p. 885.

### Experimental Procedures for Examples 6-8

Expression and purification of recombinant proteins for NMR studies. Human CR2 SCR1-2 for NMR studies was expressed in Pichia pastoris using a BioFlo 110 Fermenter (New Brunswick Scientific, Edison, N.J.) as previously described. Guthridge et al. (2001) Biochemistry 40: 5931-41. Briefly, a single colony was grown up in 5 ml Pichia basal salt medium containing 1% glycerol (BMG) overnight at 30° C. and 250 rpm, expanded to 50 ml BMG (24 hrs) and finally expanded to 300 ml BMG (24 hrs). The inoculation culture was spun down and resuspended in 30 ml BMG. The 30 ml inoculation culture was used to inoculate 1 L of minimal Pichia basal media containing 40 g of glycerol. Dissolved O₂ concentration was maintained at 40%, the temperature at 30° C. and pH at 5 using 2M KOH. Initial feeds were batch glycerol feeds; transition to methanol was eased by a methanol injection before an exponential methanol feed profile was initiated. Methanol induction lasted for two days, after which the culture was centrifuged to remove cellular debris. The supernatant was exchanged into 10 mM Formate pH 4 before being passed over a SP-Sepharose column (2×5 mL SP HiTrap columns, GE Biosciences, Pittsburgh, Pa.) followed by a CR2 affinity column, generated in house by binding GST-C3d to a GSTrap column (GE Biosciences, Pittsburgh, Pa.). CR2 was eluted with an increasing linear NaCl gradient, 0-1.0 M in ⅓× Phosphate Buffered Saline (PBS; 1.6 mM MgCl₂, 0.9 mM KCl, 0.5 mM KH₂PO₄, 45.6 mM NaCl, 2.7 mM Na₂HPO₄ pH 7.4). Purity and identity of CR2 was monitored via SDS-PAGE, western blot analysis and mass spectrometry. Both ¹⁵N and ¹⁵N-¹³C isotopically labeled proteins were prepared using this strategy. For ¹⁵N isotopically labeled CR2 ¹⁵N-Amminoum sulfate was used. For ¹⁵N-¹³C isotopically labeled CR2 ¹⁵N-amonium sulfate, ¹³C-glycerol and ¹³C-methanol were used. Isotopically labeled chemicals were purchased from Isotec Inc., Miamisburg, Ohio.

Human CR2 SCR1-2 for peptide discovery and ELISA binding studies were generated using the pMAL-P2X expression system in E. coli as previously described. Hannan et al. (2005) J Mol Biol 346:845-58. Briefly, ampicillin-resistant colonies were used to start overnight cultures that were expanded to 1 liter and grown at 37° C. until an A₆₀₀ of 0.3 was obtained. Cultures were induced with 0.3 mM isopropyl-β-D-thiogalactoside (IPTG) at 30° C. overnight before harvesting by centrifugation. Harvested pellets were resuspended in amylose column buffer (20 mM Tris-HCl, pH 7.4, 0.2 M NaCl, 1 mM EDTA) and lysed by sonication. Lysate was clarified by centrifugation and applied to an amylose resin column (New England Biosciences, Ipswich, Mass.). Bound MBP-CR2 SCR1-2 was eluted from the column using amylose column elution buffer (amylose column buffer plus 10 mM maltose). Finally, the MBP-CR2 SCR1-2 was purified by size exclusion chromatography. Purity and identity of MBP-CR2 was monitored via SDS-PAGE and western blot analysis.

Purification of EBV gp350 was completed as previously described. Guthridge et al. (2001) Biochemistry 40:5931-41. Briefly, EBV gp350 was produced by infecting Sf9 insect cells with the EBV gp350-packaged baculovirus particles (pVI-Bac Transfer vector, C-term polyhistidine tag) at an MOI of 3. The baculoviral supernatant was concentrated, buffered with 10 mM TRIS-HCl with 10 mM Imidazole pH 7.4 and applied to a 5 mL HiTrap column (GE Biosciences, Pittsburgh, Pa.) and subsequently eluted with a linear imidazole gradient. Purity and identity of EBV gp350 were monitored via SDS-PAGE and Western blot analysis.

Identification of selective peptide inhibitors. Peptides were identified using phage display libraries of either 12-mer or disulfide constrained 7-mer peptides from New England Biosciences (Ipswich, Mass.). CR2, in 0.1 M NaHCO₃ pH 8.6, was immobilized to 96 well plates overnight in a humidified container at 4° C. After washing the plate with TBST (50 mM Tris-HCl pH 7.5, 150 mM NaCl and 0.1% Tween 20), 4×10¹⁰ phage were diluted with 100 μl of TBST and added to each coated well for 60 minutes at room temperature. Bound phages were then eluted with 1 mM C3d or EBV gp350 in TBS (50 mM Tris-HCl pH 7.5, 150 mM NaCl). Approximately 1 μl of eluate was titered to determine the concentrations of phage to use for subsequent panning stages. Each additional, up to three in total, panning steps utilized TBST with 0.5% Tween 20 instead of 0.1%. After the third panning step, approximately 30 positive plaques from infected colonies were sequenced by automated DNA sequencing methods at the UCDSOM Cancer Core, to determine a consensus inhibitory peptide. The consensus sequences were synthesized as peptides using standard solid phase peptide synthesis protocols at the UCDSOM Peptide Core.

Inhibitory ELISA. Inhibition of binding was tested using a modified competition ELISA. Briefly, costar EIA/RIA 96 well half area flat bottom high binding plates (Corning Inc., Corning, N.Y.) were coated with 5 μg C3d or EBV gp350 overnight at 4° C. in ELISA coating buffer (15 mM Na₂CO₃, 35 mM NaHCO₃ pH 8.8). After coating the plate with one ligand, a potential inhibitory peptide was incubated at increasing concentrations with 2 μg MBP-CR2 in either ⅓×PBS (1.6 mM MgCl₂, 0.9 mM KCl, 0.5 mM KH₂PO₄, 45.6 mM NaCl, 2.7 mM Na₂HPO₄ pH 7.4) with C3d or PBS (4.9 mM MgCl₂, 2.7 mM KCl, 1.5 mM KH₂PO₄, 137 mM NaCl, 8.1 mM Na₂HPO₄ pH 7.4) with EBV gp350. The peptide MBP-CR2 mixture was then incubated with the ligand bound plate for one hour at 25° C. The bound MBP-CR2 was detected using a-MBP-HRP; the percent bound and thus the efficacy of inhibition by the peptide was calculated by the equation: percent inhibition=(((abs(I)−abs(N))/(abs(N))*100). Where abs(I) is the absorbance of inhibited CR2-ligand interaction and abs(N) is the absorbance of un-inhibited CR2-ligand interaction.

NMR Analysis. NMR experiments were carried out on Varian 600, 800 and 900 MHz magnets housed in the Rocky Mountain Regional NMR facility at the University of Colorado Denver—School of Medicine campus (600 and 900 MHz) and in the W. M. Keck High Field NMR Facility at the University of Colorado Boulder campus (800 MHz). The uniformly ¹⁵N/¹³C labeled SCR1-2 domains of CR2 were used to sequentially assign the ¹⁵N-TROSY-HSQC (Pervushin et al. (1997) Proc. Nat'l Acad. Sci. USA 94:12366-71) by using HNCACB (Wittkekind (1993) J Magn Reson 101:201-205) CBCA(CO)NH (Grzesiek et al. (1992) J Magn Reson 96:432-440) and ¹⁵N edited NOESY-HSQC (Zuiderweg and Fesik (1989) Biochemistry 28:2387-91) three-dimensional spectra. The NMR data were processed with nmrPipe (Delaglio et al. (1995) J Biomol NMR 6:277-93 and analysed with ccpNMR (Vranken et al. (2005) Proteins 59:687-96). Chemical shift changes were monitored using ccpNMR by overlaying TROSY-HSQC spectra from free CR2 SCR1-2 and CR2 SCR1-2 with increasing concentrations of either full length C3d or inhibitory peptides

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

All technical and scientific terms used herein have the same meaning as commonly understood to one of ordinary skill in the art to which this invention belongs unless clearly indicated otherwise.

SEQ ID NO: 1 [amino acid sequence of full-length
human CR2 protein]:
MGAAGLLGVFLALVAPGVLGISCGSPPPILNGRISYYSTPIAVGTVIRYS
CSGTFRLIGEKSLLCITKDKVDGTWDKPAPKCEYFNKYSSCPEPIVPGGY
KIRGSTPYRHGDSVTFACKTNFSMNGNKSVWCQANNMWGPTRLPTCVSVF
PLECPALPMIHNGHHTSENVGSIAPGLSVTYSCESGYLLVGEKIINCLSS
GKWSAVPPTCEEARCKSLGRFPNGKVKEPPILRVGVTANFFCDEGYRLQG
PPSSRCVIAGQGVAWTKMPVCEEIFCPSPPPILNGRHIGNSLANVSYGSI
VTYTCDPDPEEGVNFILIGESTLRCTVDSQKTGTWSGPAPRCELSTSAVQ
CPHPQILRGRMVSGQKDRYTYNDTVIFACMFGFTLKGSKQIRCNAQGTWE
PSAPVCEKECQAPPNILNGQKEDRHMVRFDPGTSIKYSCNPGYVLVGEES
IQCTSEGVWTPPVPQCKVAACEATGRQLLTKPQHQFVRPDVNSSCGEGYK
LSGSVYQECQGTIPWFMEIRLCKEITCPPPPVIYNGAHTGSSLEDFPYGT
TVTYTCNPGPERGVEFSLIGESTIRCTSNDQERGTWSGPAPLCKLSLLAV
QCSHVHIANGYKISGKEAPYFYNDTVTFKCYSGFTLKGSSQIRCKRDNTW
DPEIPVCEKGCQPPPGLHHGRHTGGNTVFFVSGMTVDYTCDPGYLLVGNK
SIHCMPSGNWSPSAPRCEETCQHVRQSLQELPAGSRVELVNTSCQDGYQL
TGHAYQMCQDAENGIWFKKIPLCKVIHCHPPPVIVNGKHTGMMAENFLYG
NEVSYECDQGFYLLGEKNCSAEVILKAWILERAFPQCLRSLCPNPEVKHG
YKLNKTHSAYSHNDIVYVDCNPGFIMNGSRVIRCHTDNTWVPGVPTCIKK
AFIGCPPPPKTPNGNHTGGNIARFSPGMSILYSCDQGYLVVGEPLLLCTH
EGTWSQPAPHCKEVNCSSPADMDGIQKGLEPRKMYQYGAVVTLECEDGYM
LEGSPQSQCQSDHQWNPPLAVCRSRSLAPVLCGIAAGLILLTFLIVITLY
VISKHRERNYYTDTSQKEAFHLEAREVYSVDPYNPAS
SEQ ID NO: 2 [amino acid sequence of SCR1 of human
CR2 protein]:
CGSPPPILNGRISYYSTPIAVGTVIRYSCSGTFRLIGEKSLLCITKDKVD
GTWDKPAPKC
SEQ ID NO: 3 [amino acid sequence of SCR2 of human
CR2 protein]:
CPEPIVPGGYKIRGSTPYR HGDSVTFACKTNFSMNGNKSVWCQANNMWG
PTRLPTC
SEQ ID NO: 4 [amino acid sequence of SCRs1 and 2
and linker-1 human CR2 protein]:
GISCGSPPPILNGRISYYSTPIAVGTVIRYSCSGTFRLIGEKSLLCITKD
KVDGTWDKPAPKCEYFNKYSSCPEPIVPGGYKIRGSTPYRHGDSVTFACK
TNFSMNGNKSVWCQANNMWGPTRLPTCVSVFPLE
SEQ ID NO: 5 [amino acid sequence of full-length
mouse CR2 protein]:
MLTWFLFYFSEISCDPPPEVKNARKPYYSLPIVPGTVLRYTCSPSYRLIG
EKAIFCISENQVHATWDKAPPICESVNKTISCSDPIVPGGFMNKGSKAPF
RHGDSVTFTCKANFTMKGSKTVWCQANEMWGPTALPVCESDFPLECPSLP
TIHNGHHTGQHVDQFVAGLSVTYSCEPGYLLTGKKTIKCLSSGDWDGVIP
TCKEAQCEHPGKFPNGQVKEPLSLQVGTTVYFSCNEGYQLQGQPSSQCVI
VEQKAIWTKKPVCKEILCPPPPPVRNGSHTGSFSENVPYGSTVTYTCDPS
PEKGVSFTLIGEKTINCTTGSQKTGIWSGPAPYCVLSTSAVLCLQPKIKR
GQILSILKDSYSYNDTVAFSCEPGFTLKGNRSIRCNAHGTWEPPVPVCEK
GCQAPPKIINGQKEDSYLLNFDPGTSIRYSCDPGYLLVGEDTIHCTPEGK
WTPITPQCTVAECKPVGPHLFKRPQNQFIRTAVNSSCDEGFQLSESAYQL
CQGTIPWFIEIRLCKEITCPPPPVIHNGTHTWSSSEDVPYGTVVTYMCYP
GPEEGVKFKLIGEQTIHCTSDSRGRGSWSSPAPLCKLSLPAVQCTDVHVE
NGVKLTDNKAPYFYNDSVMFKCDDGYILSGSSQIRCKANNTWDPEKPLCK
KEGCEPMRVHGLPDDSHIKLVKRTCQNGYQLTGYTYEKCQNAENGTWFKK
IEVCTVILCQPPPKIANGGHTGMMAKHFLYGNEVSYECDEGFYLLGEKSL
QCVNDSKGHGSWSGPPPQCLQSSPLTHCPDPEVKHGYKLNKTHSAFSHND
IVHFVCNQGFIMNGSHLIRCHTNNTWLPGVPTCIRKASLGCQSPSTIPNG
NHTGGSIARFPPGMSVMYSCYQGFLMAGEARLICTHEGTWSQPPPFCKEV
NCSFPEDTNGIQKGFQPGKTYRFGATVTLECEDGYTLEGSPQSQCQDDSQ
WNPPLALCKYRRWSTIPLICGISVGSALIILMSVGFCMILKHRESNYYTK
TRPKEGALHLETREVYSIDPYNPAS
SEQ ID NO: 6 [amino acid sequence of SCRs 1 and 2
and linker-1 mouse CR2 protein]:
EISCDPPPEVKNARKPYYSLPIVPGTVLRYTCSPSYRLIGEKAIFCISEN
QVHATWDKAPPICESVNKTISCSDPIVPGGFMNKGSKAPFRHGDSVTFTC
KANFTMKGSKTVWCQANEMWGPTALPVCESDFPLE
SEQ ID NO: 7 [amino acid sequence of inhibitory
CR2-C3d peptide (C3dp1 or C3dp1.1]:
APQXLSSQYSRT, wherein X is H or A.
SEQ ID NO: 8 [amino acid sequence of inhibitory
CR2-C3d peptide (C3dp1.3 or control)]:
APXHLSSQ, wherein X is Q or A.
SEQ ID NO: 9 [amino acid sequence of inhibitory
CR2-C3d peptide (C3dp2 or C3dp2.1)]:
ISTSNPRX₁X₂STA, wherein X₁ is H or A, and X₂ is H
or A.
SEQ ID NO: 10 [amino acid sequence of peptide
C3dp2.2]:
ISTSNPRH
SEQ ID NO: 11 [the amino acid sequence of peptide
C3dp2.3]:
IATANPRHHSTA
SEQ ID NO: 12 [the amino acid sequence of peptide
C3dcp1]:
CDPKNHVHC
SEQ ID NO: 13 [the amino acid sequence of peptide
C3dcp1.1]:
DPKNHVH
SEQ ID NO: 14 [the amino acid sequence of peptide
C3dp1]:
APQHLSSQYSRT
SEQ ID NO: 15 [the amino acid sequence of peptide
C3dp1.1]:
APQALSSQYSRT
SEQ ID NO: 16 [the amino acid sequence of peptide
C3dp1.3]:
APQHLSSQ
SEQ ID NO: 17 [the amino acid sequence of peptide
control]:
APAHLSSQ
SEQ ID NO: 18 [the amino acid sequence of peptide
C3dp2]:
ISTSNPRHHSTA
SEQ ID NO: 19 [the amino acid sequence of peptide
C3dp2.1]:
ISTSNPRAASTA
SEQ ID NO: 20 [the amino acid sequence of peptide
EBV gp350cp1]:
CSEGSLKGC

What is claimed is:

1: A non-antibody polypeptide that selectively binds to a complement receptor type 2 (CR2) protein, a ligand thereof, or both, wherein the ligand is Epstein-Barr Virus (EBV) or the 350 kilodalton Epstein-Barr Virus surface glycoprotein (EBV gp350), and wherein the non-antibody polypeptide competitively inhibits binding of EBV to a CR2 short consensus repeat (SCR) domain in a standard assay.

2. (canceled)

3: The non-antibody polypeptide of claim 1, wherein the standard assay is selected from the group consisting of competitive enzyme-linked immunosorbent assay (ELISA), radioimmunoassay (RIA), surface plasmon resonance, fluorescence activated cell sorting (FACS), flow cytometry, nuclear magnetic resonance (NMR) spectroscopy, and analytical ultracentrifugation.

4: The non-antibody polypeptide of claim 1, wherein the ligand is EBV gp350, and wherein the non-antibody polypeptide competitively inhibits binding of EBV to a CR2 short consensus repeat (SCR) domain in a competitive enzyme-linked immunosorbent assay (ELISA).

5: The non-antibody polypeptide of claim 4, wherein the CR2 SCR domain comprises an SCR1 domain of CR2, an SCR2 domain of CR2, or both, and optionally a linker between the SCR1 domain and the SCR2 domain.

6: The non-antibody polypeptide of claim 1, wherein the non-antibody polypeptide selectively binds to a binding site on EBV gp350 comprising two N-terminal 8-barrel domains (D1 and D2) and a linker region between D1-D2 (linker-1).

7: The non-antibody polypeptide of claim 6, wherein the non-antibody polypeptide selectively binds to a binding site on EBV gp350 comprising the following amino acid positions in a wildtype EBV gp350 sequence or their equivalent positions in a variant EBV gp350 sequence: E21, D22, E155, D208, E201, E210, and D296.

8: The non-antibody polypeptide of claim 7, wherein the binding site on EBV gp350 further comprises the following amino acid positions in a wildtype EBV gp350 sequence or their equivalent positions in a variant EBV gp350 sequence: Y151, I160, and W162.

9: The non-antibody polypeptide of claim 6, wherein the non-antibody polypeptide selectively binds to a binding site on EBV gp350 comprising the following amino acid positions in a wildtype EBV gp350 sequence or their equivalent positions in a variant gp350 sequence: E21, D22, E155, D208, E201, E210, D296, Y151, I160, and W162.

10: The non-antibody polypeptide of claim 1, wherein the non-antibody polypeptide selectively binds to an SCR1 domain of the CR2 protein.

11: The non-antibody polypeptide of claim 10, wherein the non-antibody polypeptide selectively binds to a binding site on the SCR1 domain of the CR2 protein comprising the following amino acid positions in a human CR2 sequence or their equivalent positions in a non-human CR2 sequence: R13, S15, A22, R28, F35, R36, K41, D49, and D52.

12: The non-antibody polypeptide of claim 1, wherein the non-antibody polypeptide selectively binds to both an SCR1 domain and an SCR2 domain of the CR2 protein.

13: The non-antibody polypeptide of claim 12, wherein the non-antibody polypeptide selectively binds to a binding site on the SCR1 domain and the SCR2 domain of the CR2 protein comprising the following amino acid positions in a human CR2 SCR sequence or their equivalent positions in a non-human CR2 SCR sequence: R13, A22, R28, F35, R36, K41, D49, D52, K57, E63, K67, Y68, R83, and R89.

14: The non-antibody polypeptide of claim 12, wherein the non-antibody polypeptide selectively binds to a binding site on the SCR1 domain and the SCR2 domain of the CR2 protein comprising the following amino acid positions in a human CR2 SCR sequence or their equivalent positions in a non-human CR2 SCR sequence: R13, A22, R28, K67, Y68, R83, G84, R89, and S103.

15: The non-antibody polypeptide of claim 1, wherein the non-antibody polypeptide inhibits the binding of the CR2 protein to EBV gp350, and wherein the non-antibody polypeptide comprises the amino acid sequence CSEGSLKGC (SEQ ID NO: 20).

16: A non-antibody polypeptide that selectively binds to a complement receptor type 2 (CR2) protein, a ligand thereof, or both, wherein the ligand is a proteolytic fragment of complement component C3 (C3 fragment), and wherein the non-antibody polypeptide competitively inhibits binding of the C3 fragments to a CR2 short consensus repeat (SCR) domain in a standard assay.

17: The non-antibody polypeptide of claim 16, wherein the standard assay is selected from the group consisting of competitive enzyme-linked immunosorbant assay (ELISA), radioimmunoassay (RIA), surface plasmon resonance, fluorescence activated cell sorting (FACS), flow cytometry, nuclear magnetic resonance (NMR) spectroscopy, and analytical ultracentrifugation.

18: The non-antibody polypeptide of claim 16, wherein the C3 fragment comprises C3d, C3dg, or iC3b.

19. (canceled)

20: The non-antibody polypeptide of claim 16, wherein the non-antibody polypeptide competitively inhibits binding of the C3 fragments to a CR2 SCR domain in a competitive enzyme-linked immunosorbant assay (ELISA) assay.

21: The non-antibody polypeptide of claim 16, wherein the non-antibody polypeptide selectively binds to both an SCR1 domain and an SCR2 domain of the CR2 protein and an inter-SCR linker.

22-35. (canceled)

36: A method of inhibiting or reducing a biological response mediated by a complement receptor type 2 (CR2) ligand in an individual comprising administering to the individual an effective amount of an non-antibody polypeptide, wherein the non-antibody polypeptide selectively binds to a CR2 protein, a ligand thereof, or both and wherein the non-antibody polypeptide competitively inhibits binding of the CR2 ligand to a CR2 short consensus repeat (SCR) domain in a standard assay.

37-43. (canceled)
